SEQUENCE LISTING (7 sequences)

SEQ ID NO: 1 - DNA, 20 bp, synthetic construct
  source 1..20: mol_type = other DNA
tgcgtcgcgccatggcggcc

SEQ ID NO: 2 - DNA, 20 bp, synthetic construct
  source 1..20: mol_type = other DNA
gatggtctgatagccggcgt

SEQ ID NO: 3 - DNA, 25 bp, synthetic construct
  source 1..25: mol_type = other DNA
atgaagcccaaactgatgtaccagg

SEQ ID NO: 4 - DNA, 18 bp, synthetic construct
  source 1..18: mol_type = other DNA
tcaaagcaggcggcaggc

SEQ ID NO: 5 - RNA, 20 nt, synthetic construct
  source 1..20: mol_type = other RNA
gtcctgatcctggcggtagt

SEQ ID NO: 6 - RNA, 20 nt, synthetic construct
  source 1..20: mol_type = other RNA
gctagtggaggggttgctcg

SEQ ID NO: 7 - RNA, 18 nt, synthetic construct
  source 1..18: mol_type = other RNA
ggaagagcgagctcttct

(19) United States
(12) Patent Application Publication
(10) Pub. No.: US 2026/0191992 A1
(43) Pub. Date: Jul. 9, 2026

(54) COMPOSITIONS AND METHODS FOR TREATING NEURODEGENERATIVE CONDITIONS

(71) Applicant: Yale University, New Haven, CT (US)

(72) Inventors: Jaime Grutzendler, New Haven, CT (US); Peng Yuan, New Haven, CT (US); Mengyang Zhang, New Haven, CT (US); Lei Tong, New Haven, CT (US)

(21) Appl. No.: 19/133,296

(22) PCT Filed: Nov. 28, 2023
(86) PCT No.: PCT/US2023/081410

§ 371 Date: May 28, 2025

Related U.S. Application Data
(60) Provisional application No. 63/428,359, filed on Nov. 28, 2022.

(51) Int. Cl. A61K 48/00; A61P 25/28; C12N 9/22; C12N 15/11; C12N 15/86

(52) U.S. Cl. CPC A61K 48/005; A61P 25/28; C12N 9/226; C12N 15/111; C12N 15/86; C12N 2310/20; C12N 2750/14143

(57) ABSTRACT

Described herein is a method of treating, ameliorating and/or preventing a neurodegenerative disease in a subject. The method includes administer to the subject a compound that reverses, ameliorates, and/or prevents a formation or enlargement of an axonal spheroid in a neuron of the subject. Also described herein is a method of reversing, ameliorating, and/or preventing a formation or enlargement of an axonal spheroid, as well as a compound for treating, ameliorating and/or preventing a neurodegenerative disease in a subject.

### CROSS-REFERENCE TO RELATED APPLICATIONS

The present application claims priority under 35 U.S.C. § 119 (e) to U.S. Provisional Patent Application No. 63/428,359, filed Nov. 28, 2022, which is incorporated herein by reference in its entirety.

### STATEMENT REGARDING FEDERALLY SPONSORED RESEARCH

This invention was made with government support under NS089734 and AG058257 awarded by National Institutes of Health. The government has certain rights in the invention.

### SEQUENCE LISTING

The ASCII text file named “047162-7344WO1 (02113)_Seq Listing.xml” created on November 15, comprising 7.10 KB Kbytes, is hereby incorporated by reference in its entirety.

### BACKGROUND

Alzheimer's disease (AD) is a neurodegenerative condition characterized by widespread disruption in neural connectivity. The extracellular deposition of the beta-amyloid (Aβ) peptide is thought to trigger a cascade of events, eventually leading to cognitive decline. However, the cellular underpinnings linking Aβ deposition and neural network disruption are not well understood, limiting the rational design of new therapies. Extensive previous work has focused on mechanisms such as cell death and synapse loss as potential causes of neural dysfunction, and therapeutic efforts have mainly centered on strategies for extracellular amyloid removal.

There is thus a need in the art for novel composition and methods that can be used to treat, ameliorate, and/or prevent neurodegenerative conditions, such as but not limited to Alzheimer's disease (AD). The present invention addresses this need.

### SUMMARY

In some aspects, the present invention is directed to the following non-limiting embodiments:

Method of Treating, Ameliorating, and/or Preventing a Neurodegenerative Condition

In some aspects, the present invention is directed to a method of treating, ameliorating, and/or preventing a neurodegenerative condition in a subject in need thereof.

In some embodiments, the method comprising administering to the subject a compound that reverses, ameliorates, and/or prevents formation or enlargement of an axonal spheroid in a neuron of the subject.

In some embodiments, the neurodegenerative condition is at least one selected from the group consisting of Alzheimer's disease, Lou Gehrig's disease (ALS), Huntington's disease, post traumatic encephalopathy, Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, and/or Lewy Body dementia.

In some embodiments, the subject is a human.

In some embodiments, the compound is a compound that downregulates an activity and/or expression level of phospholipase D3 (PLD3) in a neuron affected by the neurodegenerative condition.

In some embodiments, the compound is a compound that upregulates an activity and/or expression level of transcription factor EB (TFEB) in a neuron affected by the neurodegenerative condition.

In some embodiments, the compound is a compound that upregulates an activity and/or expression level of autophagy-related protein 5 (ATG5) in a neuron affected by the neurodegenerative condition.

In some embodiments, the compound comprises a small molecule inhibitor of PLD3.

In some embodiments, the compound comprises a protein inhibitor of PLD3.

In some embodiments, the compound comprises a nucleic acid that downregulates the expression level and/or activity of PLD3 by RNA interference, and/or an expression vector expressing the nucleic acid that downregulates the expression level and/or activity of PLD3 by RNA interference.

In some embodiments, the compound comprises a ribozyme that downregulates the expression level and/or activity of PLD3, and/or an expression vector expressing the ribozyme.

In some embodiments, the compound comprises an expression vector comprising an expression cassette. In some embodiments, the expression cassette expresses CRISPR components that downregulate the expression level and/or activity of PLD3 by CRISPR knockout or CRISPR knockdown.

In some embodiments, the compound comprises a trans-dominant negative mutant protein of PLD3, and/or an expression vector that expresses the trans-dominant negative mutant protein of PLD3.

In some embodiments the compound comprises the expression vector expressing the ribozyme.

In some embodiments the compound comprises the expression vector comprising an expression cassette expressing the CRISPR components.

In some embodiments the compound comprises the expression vector that expresses the trans-dominant negative mutant protein.

In some embodiments, the expression vector comprises a viral vector.

In some embodiments, the expression vector comprises an adeno-associated virus (AAV).

In some embodiments, the method further comprises administering to the subject a compound that removes a protein aggregate in the brain of the subject.

Method of Reversing, Ameliorating or Preventing a Formation and/or Enlargement of an Axonal Spheroid

In some the present invention is directed to a method of reversing, ameliorating or preventing a formation and/or enlargement of an axonal spheroid.

In some embodiments, the method comprises contacting a neuron affected by the formation or enlargement of the axonal spheroid with a compound that downregulates an activity and/or expression level of phospholipase D3 (PLD3) in a neuron.

In some embodiments, the method comprises contacting a neuron affected by the formation or enlargement of the axonal spheroid with a compound that upregulates an activity and/or expression level of transcription factor EB (TFEB) in a neuron.

In some embodiments, the method comprises contacting a neuron affected by the formation or enlargement of the axonal spheroid with a compound that upregulates an activity and/or expression level of autophagy-related protein 5 (ATG5) in a neuron.

In some embodiments, the axonal spheroid blocks or delays a propagation of an action potential (AP) along an axon of the neuron.

In some embodiments, the formation and/or enlargement of axonal spheroids is associated with a neurodegenerative condition in a subject.

In some embodiments, the neurodegenerative condition is at least one selected from the group consisting of Alzheimer's disease, Lou Gehrig's disease (ALS), Huntington's disease, post traumatic encephalopathy, Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, and Lewy Body dementia.

In some embodiments, the neurodegenerative condition is Alzheimer's disease, and the axonal spheroid is associated with an amyloid plaque.

In some embodiments, the compound that downregulates the expression level or the activity of PLD3 comprises a small molecule inhibitor of PLD3.

In some embodiments, the compound that downregulates the expression level or the activity of PLD3 comprises a protein inhibitor of PLD3.

In some embodiments, the compound that downregulates the expression level or the activity of PLD3 comprises a nucleic acid that downregulates the expression level and/or activity of PLD3 by RNA interference, and/or an expression vector expressing the nucleic acid that downregulates the expression level and/or activity of PLD3 by RNA interference.

In some embodiments, the compound that downregulates the expression level or the activity of PLD3 comprises a ribozyme that downregulates the expression level and/or activity of PLD3, and/or an expression vector expressing the ribozyme.

In some embodiments, the compound that downregulates the expression level or the activity of PLD3 comprises an expression vector comprising an expression cassette. In some embodiments, the expression cassette expresses CRISPR components that downregulate the expression level and/or activity of PLD3 by CRISPR knockout or CRISPR knockdown.

In some embodiments, the compound that downregulates the expression level or the activity of PLD3 comprises a trans-dominant negative mutant protein of PLD3, and/or an expression vector that expresses the trans-dominant negative mutant protein of PLD3.

### Pharmaceutical Composition

In some aspects, the present invention is directed to a pharmaceutical composition for treating a neurodegenerative condition in a subject.

In some embodiments, the pharmaceutical composition comprises a compound that downregulates an activity and/or expression level of phospholipase D3 (PLD3) in a neuron affected by the neurodegenerative condition.

In some embodiments, the pharmaceutical composition comprises a compound that upregulates an activity and/or expression level of transcription factor EB (TFEB) in a neuron affected by the neurodegenerative condition.

In some embodiments, the pharmaceutical composition comprises a compound that upregulates an activity and/or expression level of autophagy-related protein 5 (ATG5) in a neuron affected by the neurodegenerative condition.

In some embodiments, the pharmaceutical composition further comprises a pharmaceutically acceptable carrier.

In some embodiments, the neurodegenerative condition is at least one selected from the group consisting of Alzheimer's disease, Lou Gehrig's disease (ALS), Huntington's disease, post traumatic encephalopathy, Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, and Lewy Body dementia.

In some embodiments, the compound comprises a small molecule inhibitor of PLD3.

In some embodiments, the compound comprises a protein inhibitor of PLD3.

In some embodiments, the compound comprises a nucleic acid that down regulates the expression level and/or activity of PLD3 by RNA interference, and/or an expression vector expressing the nucleic acid that downregulates the expression level and/or activity of PLD3 by RNA interference.

In some embodiments, the compound comprises a ribozyme that downregulates the expression level and/or activity of PLD3, or an expression vector expressing the ribozyme.

In some embodiments, the compound comprises an expression vector comprising an expression cassette. In some embodiments, the expression cassette expresses CRISPR components that downregulate the expression level and/or activity of PLD3 by CRISPR knockout or CRISPR knockdown.

In some embodiments, the compound comprises a trans-dominant negative mutant protein of PLD3, or an expression vector that expresses the trans-dominant negative mutant protein of PLD3.

In some embodiments, the compound comprises the expression vector expressing the ribozyme.

In some embodiments, the compound comprises the expression vector comprising an expression cassette expressing the CRISPR components.

In some embodiments, the compound comprises the expression vector that expresses the trans-dominant negative mutant protein.

In some embodiments, the expression vector comprises a viral vector.

In some embodiments, the subject is a human.

In some embodiments, the pharmaceutical composition further comprises a compound that removes a protein aggregate from the subject's brain.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIGS. 1A-1B demonstrate that amyloid plaque-associated spheroids are predominantly axonal in origin, according to some embodiments. FIG. 1A is a confocal image of a coronal section of a mouse brain 4 weeks after receiving a unilateral subarachnoid injection of AAV2-GFP shows that only cell bodies on one hemisphere are GFP positive. Dashed box indicates a region of interest on the contralateral hemisphere where plaques and axons were imaged (zoomed images in FIG. 1B). FIG. 1B are zoomed images of a plaque which shows spheroid structures that can only come from transcallosal projecting axons (green) and are not associated with the dendritic marker MAP2 immunolabeling (red).

FIG. 2 is a schematic diagram for the endosomal-lysosomal-autophagic system according to some embodiments. As shown in the figure, the endosomal-lysosomal-autophagic system is a dynamic and interconnected network of membranous organelles and vesicles.

FIG. 3 is a schematic diagram for the sorting of lysosome proteins according to some embodiments. Lysosome hydrolases and enzymes are sorted either through the classic mannose-6-phosphate (M6P) pathway, or through non-M6P receptors sortilin or LIMP-2. The sorting of lysosomal membrane proteins is mediated by adaptor proteins (AP) either in a direct pathway from Golgi apparatus to early endosomes, or an indirect pathway in which the protein first gets trafficked to the plasma membrane, then endocytosed to early endosomes. In all these processes, lysosomal proteins are sorted to the limiting membrane of multivesicular bodies. The sorting of PLD3 is via the ESCRT machinery to the intralumenal vesicles of MVBs, which is a protein complex that sorts proteins destined for degradation. PLD3 is the only known lysosomal resident protein that is sorted through this pathway in mammals.

FIGS. 4A-4E demonstrate that axonal spheroids are found abundantly around amyloid plaques in AD-like mice and human AD patients, according to some embodiments.

FIG. 4A is a confocal tiling image of plaque-associated axonal spheroids (Lamp1) around amyloid plaques (Thioflavin S) in a 5×FAD mouse. FIG. 4B is a representative confocal image of axonal spheroids in a 5×FAD mouse. A single axon is labeled by GFP-expressing AAV2 virus. FIG. 4C depicts the estimation of the total number of spheroid-affected axons around individual amyloid plaques. FIGS. 4D and 4E are confocal images showing axonal spheroids labeled with PLD3 immunofluorescence around plaques in postmortem human AD brains.

FIGS. 5A-5D demonstrate that axonal spheroids show predominantly structural stability and some dynamism over extended intervals, according to some embodiments. FIG. 5A show in vivo two-photon time lapse images of PAAS, labeled with AAV2-tdTomato. The location of the amyloid plaque is indicated with dashed lines. Despite presence of PAAS, the parent axon showed no evidence of degeneration. A subset of PAAS were dynamic (arrows) and others stable (asterisk) over a two-month interval. FIG. 5B depicts the quantification of the changes in axon spheroid number at different time intervals from in vivo time lapse images of individual axons, labeled with AAV-tdTomato. Each dot indicates an axon. Dots indicated by arrows indicate observed spheroid disappearance events. FIG. 5C shows pie charts representation of data in FIG. 5B, showing the proportions of imaged axons that showed PAAS appearance, disappearance, or no change during the respective time intervals. FIG. 5D depicts the quantification of PAAS size change over time in individual axonal segments traced by in vivo imaging. Each line indicates a single axon.

FIGS. 6A-6L demonstrate that plaque-associated axonal spheroids block local action potential propagation, according to some embodiments. FIG. 6A depicts schematics of electric stimulation and two-photon calcium imaging experiments for measuring axonal conduction near spheroids. FIG. 6B depicts example of GCaMP6f-labled axons with (left panel) and without (right panel) PAAS. Plots show example traces of calcium dynamics (10 Hz imaging frame rate) in regions of interests (ROIs) at both axonal sides of PAAS. Arrows with flash icon indicate the time of stimulation. Inserts show zoomed-in plots of the calcium transients (rectangular areas). Black dotted lines indicate exponential regressions of the rising phase. The dashed lines show extrapolated spike time. FIG. 6C shows an example traces of complete conduction block at two sides of PAAS. Flash icons indicate the time of stimulation and asterisks mark the blocked calcium transients. FIG. 6D depicts the quantification of the differences in estimated spike times at the two axonal sides with respect to individual PAAS. N=10 axons without PAAS; N=21 axons with small PAAS; and N=8 axons with large PAAS, obtained from N=14 mice. Mann-Whitney tests were performed. FIG. 6E, left panel depicts the estimated probability distribution of the degree of conduction disruption in PAAS-forming axons by computational modeling (see FIGS. 7A-7F for details about simulations).

FIG. 6E, right panel shows pie charts showing percentages for different types of conduction disruption patterns observed experimentally or by computational model prediction. FIG. 6F: Schematic of the strategy for axonal electrical stimulation and two-photon voltage imaging of cell bodies to measure antidromic long-range axonal conduction. FIG. 6G: Example of voltage sensor ASAP3-labeled cell body. Blue line indicates the region of line scan (left panel) and example kymograph of two-photon line scan of ASAP3 sampled at 1 kHz, following a 10 Hz electrical stimulation (right panel). Bars under the right panel indicate stimulation and black arrows indicate action potential. FIG. 6H: Example traces generated from spatial integration of line scan images, comparing WT with 5×FAD mice. Bars indicate electrical stimulation. The electric current applied and the fast Fourier transform power (FFT) is indicated below each trace. FIG. 6I: Plot showing the probability of action potential generation (FFT power) for each cell at a defined current (individual dots). Insert shows 2 examples of the probability of action potential generation in single cells at various current stimulations, in WT and 5×FAD mice. FIG. 6J: Quantification of the currents needed for successful action potential conduction (50% probability) for each cell (individual dots) in WT and 5×FAD mice. N=25 cells from 2 WT mice; N=27 cells from 3 AD mice. Mann-Whitney tests were performed.

FIG. 6K: Quantification of the time interval between stimulation time and AP spike time in WT and 5×FAD mice. N=59 cells from 4 WT mice; N-62 cells from 4 AD mice. Mann-Whitney tests were performed. FIG. 6L: Comparison of rise times measured at the soma with either GcaMP6f or ASAP3, while stimulating contralateral axons (see FIG. 6F). Highlighted region between dashed line indicates the similarity between the approximate rise times for GcaMP6f and ASAP3.

FIGS. 7A-7F depict the computational modeling of axonal conduction abnormalities caused by PAAS, according to some embodiments. FIG. 7A depicts the computer simulations of membrane potentials recorded at two points on each side of PAAS (the arrows in upper panels) during a single action potential. Three different scenarios are presented demonstrating PAAS size-dependent conduction delays (lower panels). FIG. 7B depicts the computer simulation of membrane potentials recorded at two points on each side of PAAS (the arrows in the upper panels of FIG. 7A) during a 20 Hz stimulation train. While single action potentials can be completely blocked by larger PAAS, repetitive stimulation can eventually lead to successful conduction of the action potential due to a capacitor effect of PAAS. FIGS. 7C-7D depict the modeling of a simple resistor-capacitor electric circuit with 3 different levels of capacitance. Dashed line indicates 3 volts as an arbitrary threshold mimicking the minimal membrane potential to trigger neuronal firing. FIG. 7E depicts the representation of the simulation results with a range of spheroid diameters and membrane ion channel densities. FIG. 7F depicts the frequency distribution of the number of spheroids per individual axon (top graph) and logarithmic transformation of the diameters of individual spheroids (bottom graph) quantified from confocal images of virally labeled individual axons in 5×FAD mice, showing a gaussian distribution (D'Agostino & Pearson normality test>0.05, n=76 for bulb number and 382 for bulb size. The fitted gaussian curves, 25 and 75 percentile values are marked with red lines).

FIGS. 8A-8D demonstrate that axonal spheroids markedly disrupt spontaneous action potential conduction, according to some embodiments. FIG. 8A shows two-photon in vivo calcium imaging of spontaneous activity in axons near amyloid plaques (FSB) with and without PAAS. Given the lower frequency of spontaneously active neurons, lower frame rates were used to image larger fields of view and were thus unable to measure precisely the Ca²⁺ rise times like in FIGS. 6A-6E. FIG. 8B depicts example traces of GCaMP6s fluorescence signal obtained from ROIs (the circles) at the two sides of the plaques indicated in FIG. 8A (2 Hz imaging frame rate). Mismatched Ca²⁺ transients are indicated with orange arrows. FIG. 8C shows the correlation maps which were calculated using the average fluorescence intensity within ROI1 (left circle in FIG. 8A) as reference, and color-coded for correlation coefficient to every other pixel within the field of view. FIG. 8D depicts the quantification of the decorrelation of GCaMP6s fluorescence in ROIs at the two axonal sides with respect to the plaque, during spontaneous Ca²⁺ transients (n=12, axons without PAAS; and n=10, axons with PAAS). Mann-Whitney test was used for comparison.

FIGS. 9A-9C demonstrate that axonal spheroids lead to prolonged Ca²⁺ decay times, according to some embodiments. FIG. 9A shows Ca²⁺ transient decay time constant maps in axons with and without PAAS were measured at 3 different distances from the PAAS (as indicated by the three arrows). FIG. 9B depicts the traces of GCaMP6f fluorescence at the 3 locations (upper panel), which demonstrate that decay constants are prolonged at closer distance from PAAS (lower panel). FIG. 9C depicts the quantification of decay time constants at different distances from the PAAS. Differences between axons with PAAS at various distances from PAAS are compared with Kruskal-Wallis test. (N=12 axons and 4 ROIs per axon).

FIGS. 10A-10C demonstrate that plaque-associated axonal spheroids disrupt interhemispheric connectivity, according to some embodiments. FIG. 10A depicts the schematics of electric stimulation and two-photon calcium imaging experiments for measuring long-range axonal conduction. FIG. 10B depicts example traces of calcium dynamics (10 Hz imaging frame rate) in transcallosal axons imaged on the contralateral hemisphere. Arrows with flash icon indicate the time of stimulation. Inserts show zoomed-in plots of the calcium transients (rectangles). Black dotted lines indicate exponential regressions of the rising phase. Vertical dashed lines show extrapolated spike time. FIG. 10C depicts the quantification of the difference in stimulation time and estimated spike time in wildtype and 5×FAD mice, presented by individual axons (left panel) or mice (right panel). N=51 axons in WT mice (N=3); N=58 axons in 5×FAD mice (N=8). Mann-Whitney tests were performed for analysis by axons; and paired t-test were performed for analysis by mice.

FIGS. 11A-11F demonstrate that PAAS number correlates with severity of cognitive decline in humans, in accordance with some embodiments. FIG. 11A shows axon spheroids labeled by Amyloid Precursor Protein (APP) immunohistochemistry in post-mortem human brain (middle frontal gyrus), from subjects with mild cognitive impairment (MCI) and AD.

FIG. 11B depicts the quantification of total spheroid number around individual plaques based on confocal images of APP immunofluorescence. Each dot indicates the average from 25 plaque measurements of an individual subject. Bars indicate group average. Mann-Whitney tests were used for comparisons. N=6 MCI and 12 AD subjects. FIG. 11C: Quantification of total spheroid number around individual plaques. Each dot indicates the average of 25 plaque measurements of an individual subject. Bars indicate group average. Mann-Whitney tests were used for comparisons. N=12 AD and 6 MCI subjects. Quantification of PAAS size in AD and MCI patients based on confocal images of APP immunofluorescence. Each dot represents an average measurement of 50 PAAS. N=12 AD and 6 MCI subjects. Mann-Whitney tests were performed. FIG. 11D: Quantification of PAAS size in AD and MCI patients based on confocal images of V0A1 immunostaining. Each dot represents an average measurement of 200~250 PAAS. N=6 AD and 4 MCI patients. Mann-Whitney tests were performed. FIG. 11E lists the ApoE genotype, age, and Braak stage information of all the human brain tissue used in this study. FIG. 11F: CDR score and ABC score of human AD with PLD3 V232M variant brain tissue used in this study.

FIG. 12 illustrates that plaque-associated axonal spheroids block action potential propagation and disrupt neural connectivity, according to some embodiments. The present study demonstrated that axonal spheroids lead to blocks of action potential (AP) propagation, by functioning as electrical capacitors that act as current sinks. PAAS size is a major driver for axonal conduction defects: smaller PAAS causes fewer AP blocks, while larger PAAS causes more AP blocks. Thus, when encountered by a train of AP spikes, smaller PAAS will lead to a relative shorter “apparent conduction delay”, while larger PAAS will lead to a longer “apparent conduction delay”. In cases where all spikes are blocked, PAAS will lead to complete “apparent conduction block”. Hundreds of axons around an individual amyloid plaque develop spheroids, and these structures remain stable for extended periods of times. Therefore, given the large number of plaques present in the AD brain, PAAS could significantly affect neural networks by widespread disruption of axonal connectivity. In humans the plaque density is lower than in mice, however, the lengths of axons are much greater, thereby increasing the probability of adjacency to plaques and development of PAAS. In regions such as hippocampus, where parallel compact axonal bundles follow a stereotyped projection path along a tri-synaptic loop, a single amyloid plaque could be especially detrimental since it can lead to the development of more spheroids. The tri-synaptic circuit within hippocampus, as well as long-range connections between hippocampus and other regions in the cortex, which are all critical in memory formation, could be particularly vulnerable to conduction disruption caused by PAAS. In addition, PAAS can also lead to disruption in Hebbian synaptic plasticity, due to the requirement of precise timing of firing between pre-synaptic and post-synaptic terminals. Altogether, action potential blocks caused by PAAS could be detrimental to various neural processes such as memory formation and reaction time, potentially contributing to cognitive decline in AD.

FIGS. 13A-13H demonstrate that the accumulation of abnormally enlarged multivesicular bodies is associated with spheroids expansion and disease progression in AD-like mice, according to some embodiments. FIG. 13A shows confocal image of PAAS in a 5×FAD mouse brain showing a prominent halo of spheroids labeled by anti-LAMP-1 immunohistochemistry (Lamp1) around an amyloid plaque (ThioflavinS). Right panel shows the zoomed-in picture from the PAAS labeled with the white dashed box. Arrows indicate enlarged LAMP1-positive multivesicular bodies (MVBs). FIG. 13B depicts the quantification of large MVB occurrence within PAAS at different ages in 5×FAD mice. N=3 mice for each age group. Each dot represents average measurements from 200 to 500 individual PAAS. Kruskal-Wallis test was performed. FIG. 13C depicts the quantification of PAAS areas with and without enlarged MVBs. N=3 mice from each group. Each pair of dots represents average measurements from 50 to 100 individual PAAS in the same mouse. Paired t-test was performed. FIG. 13D upper panel is the electron microscopy images of PAAS (the area enclosed by solid line) in a 5×FAD mouse brain. FIG. 13D, lower panel shows two examples of zoomed-in images of MVBs (dashed boxes) from the image of the upper panel. FIG. 13E shows confocal images of PAAS with high and low Cathepsin D contents. White dotted lines mark the perimeters of PAAS. FIG. 13F depicts the quantification of spheroid size as a function of Cathepsin D immunoreactivity levels. N=6 mice for each group; each pair of dots represents average measurement from 50 PAAS in the same mouse. Paired t-test was performed. FIG. 13G is a confocal image of PAAS expressing the pH sensor SEpHluorin-mCherry in a 5×FAD mouse brain. FIG. 13H depicts the quantification of PAAS size as a function of pH. Neutral and acidic pH are defined by a threshold of red-green fluorescence ratio of 0.5. N=4 mice for each group; each pair of dots represents the average measurement from 50 PAAS in the same mouse. Paired t-test was performed.

FIGS. 14A-14I demonstrate that abnormally enlarged multivesicular body accumulation is associated with spheroids expansion and cognitive decline in human AD patients, according to some embodiments. FIG. 14A shows confocal image of axonal spheroids (labeled by V0A1 (ATPase H+ Transporting V0 Subunit A1) around an amyloid plaque (ThioflavinS), in a post-mortem human AD brain. Panel on the right show zoomed-in example image of PAAS in the white dashed box, with large MVBs indicated by arrows. FIG. 14B shows a confocal image of an individual PAAS with enlarged V0A1-positive MVBs, indicated by the arrows. FIG. 14C depicts the quantification of PAAS size as a function of presence of enlarged MVBs. N=4 subjects from each group. Each dot represents the average measurements of 200 to 500 individual PAAS. Paired t-test was performed. FIG. 14D shows confocal images of PAAS labeled by APP and Cathepsin D immunohistochemistry in a post-mortem human AD brain. Right panels show zoomed-in examples of PAAS with low or high Cathepsin D contents. White dotted lines indicate the outlines of PAAS. FIG. 10E depicts the quantification of PAAS size as a function of Cathepsin D contents. N=11 human subjects. Each pair of dots represents average measurements from 50 PAAS in the same postmortem brain. Paired t test was performed. FIGS. 14F-14H depicts the comparisons of PAAS features between AD and MCI patients (FIG. 14F, PAAS area; FIG. 14G, MVB occurrence; and FIG. 14H, Cathepsin D contents). Each dot represents average measurement from 50 PAAS in (FIG. 14F) and (FIG. 14H), 200~500 PAAS in (FIG. 14G). N=12 AD and 6 MCI subjects in (FIGS. 14F and 14H); N=3 AD and 3 MCI subjects in FIG. 14G. Mann-Whitney tests were performed. FIG. 14I depicts the receiver operating characteristic (ROC) curves clearly differentiate AD from MCI patients using PAAS diameter and APP or Cathepsin D contents as parameters.

FIGS. 15A-15B demonstrate that PLD3 accumulates in the intralumenal vesicles of MVBs within axonal spheroids, according to some embodiments. FIG. 15A shows PLD3 immunohistochemistry (“PLD3”), which shows marked enrichment in PAAS in human postmortem AD and 5×FAD brain tissue. FIG. 15B shows confocal (the upper panel) and expansion microscopy (the lower pandel) images of PLD3 immunohistochemistry (“PLD3”) and virally-labeled LAMP1-GFP (“Lamp1-GFP”) in PAAS. Arrows indicate PLD3 puncta in enlarged LAMP1-positive multivesicular bodies (MVBs).

FIGS. 16A-16D demonstrate that no PLD3 protein expression in microglia or astrocytes in 5×FAD mice or human AD brain, according to some embodiments. FIGS. 16A-16B are confocal images showing absence of PLD3 signal (“PLD3”) within Ibal-labeled microglia (“Ibal”) in 5×FAD mouse brain (FIG. 16A) and postmortem brain tissue of AD human patients (FIG. 16B). FIG. 16C shows confocal imaging of 5×FAD mouse brain showing absence of PLD3 signal (“PLD3”) within S100-labeled astrocyte (“S100”). FIG. 16D shows confocal imaging of human AD postmortem brain tissue showing absence of PLD3 signal (“PLD3”) within ALDHIL1-labeled astrocyte (“ALDHIL1”).

FIGS. 17A-17L demonstrate that PLD3 mediates multivesicular body enlargement and spheroid expansion, according to some embodiments. FIG. 17A shows confocal images of PAAS in 10-month-old 5×FAD mice with AAV2-mediated PLD3 (left panel) or control GFP (right panel) overexpression. Right panels show zoomed-in examples. FIG. 17B depicts the quantification of PAAS area in 10-month-old 5×FAD mice with AAV2-mediated PLD3 or control GFP overexpression. N=3 and 5 mice for GFP and PLD3 groups, respectively. Each dot represents average measurements from 350 to 600 individual PAAS. Mann-Whitney tests were performed. FIG. 17C shows confocal images of adjacent PAAS with (left dashed line) and without (right dashed line) PLD3 overexpression. Arrows indicate enlarged MVBs. FIG. 17D depicts the quantification of enlarged MVB occurrence in PAAS of 10-month-old 5×FAD mice with PLD3 or control GFP overexpression. N=3 and 4 mice for GFP and PLD3 groups, respectively. Each dot represents average measurement from 150 to 200 individual PAAS. Mann-Whitney tests were performed. FIG. 17E shows confocal images of PAAS in 5×FAD mice with PLD3 or control GFP overexpression. Arrows indicate enlarged MVBs. FIG. 17F depicts the quantification of MVB size in PAAS of 10-month-old 5×FAD mice with PLD3 or control GFP overexpression. N=3 and 4 mice for GFP and PLD3 group, respectively. Each dot represents average measurement from 500 to 1000 MVBs. Mann-Whitney tests were performed. FIG. 17G shows confocal images of virus infected (“GFP”) and uninfected (“Lamp1”) PAAS in 5-month-old 5×FAD mice with PLD3 (right two panels) or control GFP (left two panels) overexpression. FIG. 17H depicts the quantification of PAAS sizes in 5-month-old 5×FAD mice with PLD3 or GFP overexpression. N=6 and 5 mice for PLD3 and GFP groups, respectively. Each dot represents average from 350~600 PAAS measurements. Mann-Whitney tests were performed. FIG. 17I shows zoomed-in example images of PAAS with and without PLD3 overexpression. The dash lines mark the outline of individual PAAS. Arrows indicated enlarged MVBs. FIG. 17J depicts the quantification of large MVBs occurrence in PAAS in 5-month-old 5×FAD mice with PLD3 or GFP overexpression. N=4 mice for each group. Each dot represents average measurement from 150~250 PAAS. Mann-Whitney tests were performed. FIGS. 17K-17L depicts the quantification of plaque number (FIG. 17K) and size (FIG. 17L) in mice with GFP or PLD3 overexpression. N=5 and 6 for GFP and PLD3 groups, respectively. For FIG. 17L, each dot represents average from 100~250 plaque measurements. Unpaired t-tests were performed.

FIGS. 18A-18J demonstrate that Aβ facilitates PLD3-induced MVB enlargement and spheroids expansion, according to some embodiments. FIG. 18A show confocal images of LAMP1-positive vesicular structures in PAAS and cell bodies in 10-month-old mice with PLD3 overexpression. FIG. 18B depicts the quantification of MVB sizes in groups described in FIG. 18A. N=4 mice for each group. Each dot represents average measurement from 500~1000 MVBs from PAAS or 100-200 LAMP1-positive vesicles from cell bodies. Welch's t-test was used. FIG. 18C shows confocal images of spheroids and LAMP1-positive vesicles in wildtype mice overexpressing PLD3. FIG. 18D shows confocal (left panel) and expansion microscopy (right panel) images of Aβ42 immunohistochemistry (“Abeta 42”) and virally-labeled LAMP1-GFP (“Lamp1-GFP”) in PAAS. Arrows indicate Aβ42 puncta in enlarged LAMP1-positive MVBs. FIG. 18E depicts confocal images of FM1-43 dye (endocytosis marker) incorporation into PAAS in cultured brain slices following vehicle or PitStop2 (endocytosis inhibitor) treatment. FIG. 18F depicts the quantification of FM1-43 incorporation into PAAS with PitStop2 or Dynasore treatment. N=20 PAAS for PitStop2 or Dynasore at different concentrations. Data are represented as mean±S.E.M. Red dash lines show regression to a sigmoid inhibition curve. F-tests were used to compare the fitted top and bottom parameters for each group. FIG. 18G shows schematics of in vivo assay of intra parenchymal brain microinjections of fluorescently labeled Aβ-42 peptide for measuring Aβ endocytosis into PAAS. FIGS. 18H-18I show confocal images of injected fluorescently tagged Aβ-42 (“Injected Aβ”) incorporated into PAAS (“Lamp1”). The dashed lines indicate the outline of PAAS based on LAMP-1 immunohistochemistry. Arrows point to Aβ-42 puncta. FIG. 18J depicts the quantification of Aβ-42 incorporation in PAAS. N=3 mice, each with average measurements from 10 field of view, Wilcoxon matched-pairs signed rank tests were used to compare between groups.

FIGS. 19A-19L demonstrate that CRISPR/Cas9-mediated PLD3 deletion reduces PAAS pathology, according to some embodiments. FIG. 19A show schematics of two guide RNAs targeting the PLD3 gene. FIG. 19B shows confocal images of adjacent PAAS with (GFP positive) and without (GFP negative) PLD3 deletion. The dashed lines mark the outlines of individual PAAS. Arrows indicate enlarged MVBs. FIG. 19C depicts the quantification of MVB occurrence in PLD3-deleted and control PAAS in 10-month-old 5×FAD/LSL-Cas9 mice. N=4 mice for each group. Each dot is the average of 150 to 250 individual PAAS measurements. Mann-Whitney tests were performed. FIG. 19D shows confocal images of PAAS expressing control scrambled sgRNAs (left panel) or PLD3-targeting sgRNAs (right panel) in 5×FAD/LSL-Cas9 mice, showing infected (GFP positive) and uninfected (LAMP-1 immunohistochemistry) PAAS near a plaque (“ThioflavinS”). FIG. 19E depicts the quantification of PAAS sizes in 10-month-old mice with or without PLD3 deletion. N=6, 6, 4 mice for control sgRNA, PLD3 sgRNA-1 and PLD3 sgRNA-2, respectively. Each dot represents the average of 350 to 600 individual PAAS measurements. Mann-Whitney tests were performed. FIGS. 19F-19G shows confocal images of infected and uninfected PAAS in 5×FAD mice with control scrambled sgRNA (FIG. 19F) or PLD3 targeted sgRNA (FIG. 19G). FIG. 19H depicts the quantification of PAAS area in 5-month-old mice with control sgRNA or PLD3 sgRNA 2. N=4 and 5 mice for control and PLD3 sgRNA groups, respectively. Each dot represents average from 350~600 PAAS measurements. Mann-Whitney tests were performed. FIG. 19I shows confocal images of adjacent PAAS with (GFP positive, upper dashed lines) and without (GFP negative, lower dashed lines) PLD3 deletion. Arrows indicate enlarged MVBs. FIG. 19J depicts quantification of MVBs occurrence in 5-month-old mice with control sgRNA or PLD3 sgRNA 2. N=4 mice for each group. Each dot represents average from 150~250 PAAS measurements. Mann-Whitney tests were performed. FIGS. 19K-19L depict the quantification of plaque number (FIG. 19K) and size (FIG. 19L) in mice with control or PLD3 sgRNA. N=5 mice for each group. Each dot represents average from 100~250 plaque measurements. Unpaired t-tests were performed. The sequences listed in FIG. 19A are PLD3 sgRNA 1 (GTCCTGATCCTGGCGGTAGT, SEQ ID NO:5) and PLD3 sgRNA 2 (GCTAGTGGAGGGGTTGCTCG, SEQ ID NO:6).

FIGS. 20A-20F show the validation of CRISPR/Cas9-mediated PLD3 deletion, according to some embodiments. FIGS. 20A-20D are confocal images of PLD3 immunohistochemistry in tissue infected (“GFP”) and uninfected with PLD3-targeted sgRNA1 (FIGS. 20A and 20C) or sgRNA2 (FIGS. 20B and 20D). Circular dashed lines indicate outlines of infected cell bodies or individual spheroids. Straight dashed lines indicate zoomed-in field of views on the right. FIGS. 20E-20F depict the quantifications of PLD3 fluorescence intensities in cell bodies with (GFP+) or without (GFP-) PLD3-targeted sgRNA1 (FIG. 20E) or sgRNA2 (FIG. 20F). 15~25 cell bodies were measured from each group. Mann-Whitney tests were performed.

FIGS. 21A-21I demonstrate that CRISPR/Cas9-mediated PLD3 deletion improves axonal conduction, according to some embodiments. FIG. 21A shows schematics of calcium imaging to measure conduction in contralateral axons with or without PLD3 manipulation in 5×FAD/LSL-Cas9 mice. FIG. 21B depicts example traces of calcium dynamics (20 Hz imaging frame rate) in axons on the contralateral hemispheres following PLD3 deletion with sgRNA 1. Arrows with flash icon indicate the time of stimulation. Inserts show zoomed-in plots of the calcium transients (rectangular blocks). Black dotted lines indicate exponential regressions of the rising phase. Vertical dashed lines show extrapolated spike time. FIG. 21C depicts the quantification of the difference in stimulation time and estimated spike time in PLD3-deleted and control axons in 5×FAD/LSL-Cas9 mice, presented by individual axons (left panel) or by mice (right panel). N=71 manipulated and 46 control axons, from N=5 mice. Mann-Whitney tests were performed for analysis by axons; paired t-test were performed for analysis by mice. FIG. 21D depicts example traces of calcium dynamics (20 Hz imaging frame rate) in contralateral axons following PLD3 deletion with sgRNA-2. Arrows with flash icon indicate the time of stimulation. Inserts show zoomed-in plots of the calcium transients (rectangular blocks). Black dotted lines indicate exponential regressions of the rising phase. Verticle dashed lines show extrapolated spike times. FIG. 21E depicts the quantification of the spike times in PLD3-deleted and control axons in 5×FAD/LSL-Cas9 mice, shown by either individual axons (left panel) or individual mice (right panel). N=80 manipulated and 61 control axons, from N-6 mice. Mann-Whitney tests were performed for analysis by axons; paired t-test were performed for analysis by mice. FIG. 21F depicts example traces of calcium dynamics (20 Hz imaging frame rate) in contralateral axons following PLD3 overexpression. Arrows with flash icon indicate the time of stimulation. Inserts show zoomed-in plots of the calcium transients (rectangular blocks). Black dotted lines indicate exponential regressions of the rising phase. Vertical dashed lines show extrapolated spike times. FIG. 21G depicts the quantification of the spike times in PLD3-overexpressed and control axons in 5×FAD mice, shown by either individual axons (left panel) or individual mice (right panel). N=69 manipulated and 49 control axons, from N=5 mice. Mann-Whitney tests were performed for analysis by axons; paired t-test were performed for analysis by mice. FIG. 21H depicts the quantification of the difference in stimulation time and estimated spike time in axons with scrambled sgRNA and control GCaMP only axons in 5×FAD/LSL-Cas9 mice, presented by individual axons (left panel) or by mice (right panel). N-69 manipulated and 37 control axons, from N=4 mice. Mann-Whitney tests were performed for analysis by axons; paired t-test were performed for analysis by mice. FIG. 21I depicts the quantification of the difference in stimulation time and estimated spike time in axons with dTomato overexpression and control GCaMP only axons in 5×FAD mice, presented by individual axons or by mice. N=42 manipulated and 21 control axons, from N=4 mice. Mann-Whitney tests were performed for analysis by axons; paired t-test were performed for analysis by mice.

FIGS. 22A-22H demonstrate that overexpression of transcription factor EB (TFEB), autophagic-related protein 5 (ATG5), but not Beclin1 or progranulin, ameliorate PAAS pathology. FIG. 22A shows confocal images of PAAS in 5-month-old 5×FAD mice with AAV2-mediated TFEB (left panels) or control GFP (right panels) overexpression. FIG. 22B depicts the quantification of PAAS area in 5-month-old 5×FAD mice with AAV2-mediated TFEB or control GFP overexpression. N=5 and 6 mice for GFP and TFEB groups, respectively. Each dot represents average measurements from 350 to 600 individual PAAS. Mann-Whitney tests were performed. FIG. 22C depicts example traces of calcium dynamics (20 Hz imaging frame rate) in contralateral axons following TFEB overexpression. Arrows with flash icon indicate the time of stimulation. Inserts show zoomed-in plots of the calcium transients (rectangular blocks). Black dotted lines indicate exponential regressions of the rising phase. Vertical dashed lines show extrapolated spike times. FIG. 22D depicts the quantification of the spike times in TFEB-overexpressed and control axons in 5×FAD mice, shown by either individual axons (left panel) or individual mice (right panel). N=32 manipulated and 21 control axons, from N=4 mice. Mann-Whitney tests were performed for analysis by axons; paired t-test were performed for analysis by mice. FIG. 22E shows confocal images of PAAS in 5-month-old 5×FAD mice with AAV2-mediated ATG5 (upper panels) or control GFP (lower panels) overexpression. Right panels show zoomed-in examples. FIG. 22F depicts the quantification of PAAS area in 5-month-old 5×FAD mice with AAV2-mediated ATG5 or control GFP overexpression. N=5 and 7 mice for GFP and ATG5 groups, respectively. Each dot represents average measurements from 350 to 600 individual PAAS. Mann-Whitney tests were performed. FIGS. 22G-22H depict the quantification of PAAS area in 5-month-old 5×FAD mice with AAV2-mediated BECN1, GRN or control GFP overexpression. N=5, 3 and 6 mice for GFP, BECN1 and GRN groups, respectively. Each dot represents average measurements from 350 to 600 individual PAAS. Mann-Whitney tests were performed.

FIG. 23 depicts the proposed model of PAAS enlargement and functional consequences in Alzheimer's disease, in accordance with some embodiments. 1) The present study demonstrated that the accumulation of abnormally enlarged MVBs is a major driver of PAAS enlargement. Small PAAS predominately contain mature lysosomes, while bigger PAAS contain abundant and enlarged MVBs. 2) The present study identified PLD3 as a critical modulator of MVB abnormalities and subsequent spheroid enlargement. PLD3 is uniquely sorted through the ESCRT pathway into the intralumenal vesicles (ILVs) of MVBs. Accumulation of PLD3 at spheroids could lead to MVB enlargement by interfering with ESCRT machinery. This process could be exacerbated with the presence of Aβ. Aβ from extracellular amyloid deposits is actively endocytosed and is present in the same subcellular compartments as PLD3. PLD3 could thus work synergistically with Aβ, leading to greater MVB abnormalities. 3) Large PAAS cause more severe conduction blocks, by functioning as electrical capacitors that act as current sinks. Given that hundreds of axons around each plaque develop spheroids and these structures remain stable for extended periods of times, the large number of plaques present in the AD brain could significantly affect neural networks by widespread disruption of axonal connectivity. 4) The present study found that cortical neurons in 5×FAD mice exhibited hyperactivity, and this can be corrected by restoring axon conduction through reducing PAAS in basal forebrain cholinergic projections. This suggests that PAAS can cause widespread disruption of neural circuit function. In addition, parallel compact axonal bundles that follow a stereotyped projection path along a tri-synaptic loop in hippocampus, a region critical for memory formation, could be particularly vulnerable to amyloid plaques located in the region. Furthermore, neural processes that rely on temporally precise long-range coordination among brain regions, such as memory consolidation, could be severely affected. In addition, synaptic plasticity could also be disrupted, due to the requirement of precise timing of firing between pre-synaptic and post-synaptic terminals. Altogether, action potential blocks caused by PAAS could be detrimental to various neural processes such as memory formation and reaction time, potentially contributing to cognitive decline in AD.

FIGS. 24A-24G demonstrates that the reduction in axonal spheroids by PLD3 deletion improves neural circuit function, in accordance with some embodiments. FIG. 24A: Schematics showing cholinergic neurons in the basal forebrain projecting to the cortex following infection with AAV viruses encoding either PLD3 or control sgRNAs (left panel), and two photon images show intermingled projecting axons from basal forebrain (“tdTomato”) with GCaMP6f-labeled cortical neurons (right panel). Calcium imaging was performed in cortical neurons of awake mice in the same region as the projecting forebrain axons which are likely cholinergic. FIG. 24B: Representative two-photon image of GCaMP6f-labeled cortical neurons. FIG. 24C: Example of raw calcium traces from selected individual cortical neurons. FIG. 24D: Quantification of spike counts from individual neurons during a 30-minute imaging session. Each dot represents the average spike count from all cells in the same mouse. Bars indicate group mean. Violin plots show distributions of spike counts from all individual neurons from the same group. FIG. 24E: Quantification of pair-wise mutual information grouped by distances between neurons. Two-way ANOVA test was used to compare between groups. FIG. 24F: Quantification of neurons classified in clusters by their activity patterns (Louvian clustering, see methods) and represented by cluster size distribution. FIG. 24G: Quantification of population entropy (as a measurement of temporal variance of the firing pattern) from each mouse imaged. For FIGS. 24D, 24E and 24G, N=4, 4, and 6 mice in wildtype group, 5×FAD with control sgRNA group and 5×FAD with PLD3 sgRNA group, respectively. For FIG. 24F, N=67, 21, and 45 clusters in wildtype group, 5×FAD with control sgRNA group and 5×FAD with PLD3 sgRNA group, respectively. For FIGS. 24D, 24F and 24G, One-way ANOVA test was used to compare among groups and p-values indicating the post-hoc comparison between groups, with Sidak's correction for multiple comparison.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed. Despite continuing debates about the amyloid cascade hypothesis, emerging evidences support the concept that an imbalance between production and clearance of Aβ42 and related Aβ peptides is a very early, often initiating factor in Alzheimer's disease. In the amyloid cascade hypothesis, Aβ is the main driver for the pathogenesis of the disease. Increased Aβ42 production and accumulation lead to multiple downstream pathological effects, including synaptic dysfunction, neurofibrillary tangle formation, neuronal loss, glia activation and inflammation, which contribute to cognitive impairment in AD (Selkoe, D. J et al., EMBO Mol Med 8, 595-608).

Among the many disease-modifying therapies that have been tested in clinical trials for AD, β-amyloid is the most common target for drug development. Multiple antibodies have been developed to target either Aβ oligomers or amyloid plaques. However, despite tremendous effort, limited success has been achieved (Long et al., Cell 179, 312-339, and Scheltens et al. Lancet 397, 1577-1590). In June 2021, Aducanumab, a monoclonal antibody that targets aggregated forms of Aβ, has been approved by the Food and Drug Administration (FDA). This decision has sparked heated controversy among the science community (Mullard, Nat Rev Drug Discov 20, 496, and Lancet Neurol 20, 585), due to questions regarding its efficacy and side effects. The failure of anti-amyloid therapies highlighted that the Alzheimer field is in great need for novel therapies.

The study described herein (also referred as “the present study”) indicates that the failures of the anti-amyloid therapies are, at least partially, due to the failure of these therapies to target pathological changes downstream of the Aβ oligomers or amyloid plaques. Currently, most of the clinical trials recruit patients with mild to moderate symptomatic AD. These are stages at which amyloid has already caused significant downstream pathological changes, including synaptic dysfunction, neuronal loss and axonal spheroids. Thus, the removal of amyloid per se might not be as effective.

Using Alzheimer's disease as a model, the present study identified axonal spheroids as prominent contributors to neural network dysfunction in the neurodegenerative disease.

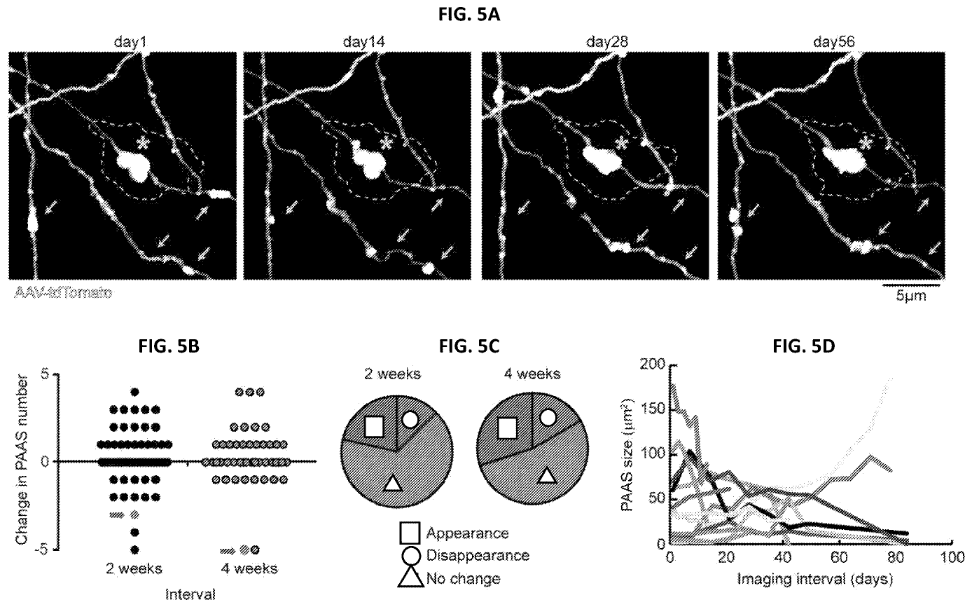

Axonal spheroids are a pathology found in AD, as well as in various other neurological disorders (Li et al., Exp Neurol 246, 62-71, Coleman et al., Nature reviews. Neuroscience 6, 889-898, Leigh et al., Brain 112 (Pt 2), 521-535, Oyanagi et al., Brain Pathol 27, 748-769, and Hill et al., Trends Neurosci 39, 311-324), such as Lou Gehrig's disease (ALS), Huntington's disease, post traumatic encephalopathy, Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, and Lewy Body dementia. Axonal spheroids have been discovered for more than a century and were referred to as “neuritic dystrophies,” due to that early studies mistook the structures for degenerating, retracting neurites. These structures appear to be all axonal rather than dendritic in origin, and develop in the vicinity of amyloid plaques in the case of AD (See e.g., FIGS. 5A-5B). As such, these structures are sometimes referred to as plaque-associated axonal spheroids or “PAAS” herein. Although various hypotheses regarding the development of PAAS have been proposed through the years, these structures have not been a major focus of therapeutics and their pathophysiological significance remains uncertain.

The present study demonstrated that enlarging axonal spheroids, by acting as electric current sinks, cause local action potential (AP) blockades in a size dependent manner. These local AP blockades lead to severe disruption in long-range connectivity. Since hundreds of PAAS develop around each individual amyloid plaque and remain stably incorporated into neural circuits for extended periods of time, the large number of plaques present in the AD brain significantly affect neural networks by widespread disruption of axonal connectivity.

The present study discovered that the formation and enlargement of PAAS is an aberrant sprouting and outgrowth process that resembles growth cone formation during neural development, which involves the neuronal endolysosomal protein phospholipase D3 (PLD3). According to a model based on the present study, the enlargement of spheroid is driven by the accumulation of endolysosomes, including PLD3-induced aberrantly enlarged multivesicular bodies (MVBs). The enlarged spheroids cause axonal conduction blockades, leading to severe disruption in long-range connectivity, potentially affecting neural networks and cognitive function.

Using CRISPR/Cas9-mediated deletion and viral-mediated overexpression as examples, the present study demonstrated that the down-regulation of PLD3 reduces the abundance of enlarged MVBs within spheroids, decreases PAAS size and restores interhemispheric connectivity, while the up-regulation of PLD3 has the opposite effects.

Using AAV2-mediated overexpression as an example, the present study shows that the up-regulation of transcription factor EB (TFEB) and autophagic-related protein 5 (also referred to as autophagy related 5, ATG5) ameliorate PAAS pathology.

In accordance with the above, the instant specification is, among others, directed to the following embodiments.

In some aspects, the instant specification is directed to a method of reversing or preventing formation or an enlargement of an axonal spheroid in a neuron.

In some aspects, the instant specification is directed to a method of treating, ameliorating and/or preventing a neurodegenerative disease in a subject in need. The neurodegenerative disease includes but not limited to Alzheimer's disease, Lou Gehrig's disease (ALS), Huntington's disease, post traumatic encephalopathy, Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, and/or Lewy Body dementia.

In some aspects, the instant specification is directed to a composition for treating, ameliorating and/or preventing a neurodegenerative disease in a subject in need.

It is worth noting that, although the instant specification describes Alzheimer's disease as a non-limiting illustrative example, other types of neurodegenerative conditions are specifically included in the scope of the instant specification. One of ordinary skill in the art would understand that the prominent axonal pathology which includes the formation of large axonal enlargements (i.e., the spheroids) exists in other types of neurodegenerative conditions, such as but not limited to Lou Gehrig's disease (ALS), Huntington's disease, post traumatic encephalopathy, lysosomal storage disorders including Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, and/or Lewy Body dementia, as well (see e.g., Li et al., Exp Neurol 246, 62-71 (2013), Coleman et al., Nature reviews. Neuroscience 6, 889-898 (2005), Leigh et al., Brain 112 (Pt 2), 521-535 (1989), Oyanagi et al., Brain Pathol 27, 748-769 (2017) and Hill, Trends Neurosci 39, 311-324 (2016)). As such, one of ordinary skill in the art would expect that the methods and compounds as described herein are applicable to other types of neurodegenerative conditions.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of +20% or +10%, in certain embodiments+5%, in certain embodiments+1%, in certain embodiments+0.1% from the specified value, as such variations are appropriate to perform the disclosed methods.

A “disease” is a state of health of an animal wherein the animal cannot maintain homeostasis, and wherein if the disease is not ameliorated then the animal's health continues to deteriorate.

A “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

In one aspect, the terms “co-administered” and “co-administration” as relating to a subject refer to administering to the subject a compound and/or composition of the disclosure along with a compound and/or composition that may also treat or prevent a disease or disorder contemplated herein. In certain embodiments, the co-administered compounds and/or compositions are administered separately, or in any kind of combination as part of a single therapeutic approach. The co-administered compound and/or composition may be formulated in any kind of combinations as mixtures of solids and liquids under a variety of solid, gel, and liquid formulations, and as a solution.

As used herein, the term “pharmaceutical composition” or “composition” refers to a mixture of at least one compound useful within the disclosure with a pharmaceutically acceptable carrier. The pharmaceutical composition facilitates administration of the compound to a patient. Multiple techniques of administering a compound exist in the art including, but not limited to, subcutaneous, intravenous, oral, aerosol, inhalational, rectal, vaginal, transdermal, intranasal, buccal, sublingual, parenteral, intrathecal, intragastrical, ophthalmic, pulmonary, and topical administration.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the patient. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, a “pharmaceutically effective amount,” “therapeutically effective amount,” or “effective amount” of a compound is that amount of compound that is sufficient to provide a beneficial effect to the subject to which the compound is administered.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the terms “subject” and “individual” and “patient” can be used interchangeably and may refer to a human or non-human mammal or a bird. Non-human mammals include, for example, livestock and pets, such as ovine, bovine, porcine, canine, feline and murine mammals. In certain embodiments, the subject is human.

As used herein, the term “treatment” or “treating” is defined as the application or administration of a therapeutic agent, i.e., a compound useful within the disclosure (alone or in combination with another pharmaceutical agent), to a patient, or application or administration of a therapeutic agent to an isolated tissue or cell line from a patient (e.g., for diagnosis or ex vivo applications), who has a disease or disorder and/or a symptom of a disease or disorder, with the purpose to cure, heal, alleviate, relieve, alter, remedy, ameliorate, improve or affect the disease or disorder and/or the symptoms of the disease or disorder. Such treatments may be specifically tailored or modified, based on knowledge obtained from the field of pharmacogenomics.

Method of Treating, Ameliorating, and/or Preventing Neurodegenerative Condition

As detailed elsewhere in the instant specification, the present study demonstrated that the formation and enlargement of axonal spheroids disrupt the propagation of action potentials (APs) along the axons of neurons. Using Alzheimer's disease as a model, the present study demonstrated that axonal spheroids associated with amyloid plaques (also referred to as “plaque-associated axonal spheroids” or “PAAS” herein) disrupt the propagation of action potentials by acting as electric current sinks.

The present study demonstrated various methods to reverse or prevent the formation or enlargement of the axonal spheroids in the neurons, which include: inhibiting the accumulation of multivesicular bodies (MVBs) in the axon, such as by down-regulating PLD3; increasing the autophagic activity and the lysosomal activity, such as by upregulating transcription factor EB (TFEB); increasing the autophagic activity, such as by upregulating autophagy-related protein 5 (ATG5).

The present study further demonstrated that reducing the sizes of the axonal spheroids restores the axonal conduction properties of the axons affected by the axonal spheroids.

Therefore, in some embodiments, the instant specification is directed to a method of treating ameliorating, and/or preventing a neurodegenerative condition in a subject in need thereof. In some embodiments, the method includes administering to the subject an effective amount of a compound that reverses, ameliorates, and/or prevents a formation or enlargement of an axonal spheroid in a neuron of the subject.

In some embodiments, the method restores a propagation of an action potential (AP) along an axon of a neuron blocked or delayed by an axonal spheroid on the axon.

In some embodiments, the neurodegenerative disease is Alzheimer's disease, Lou Gehrig's disease (ALS), Huntington's disease, post traumatic encephalopathy, lysosomal storage disorders including Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, Lewy Body dementia, or combinations thereof. In some embodiments, the neurodegenerative disease is Alzheimer's disease. In some embodiments, the subject is a mammal. In some embodiments, the mammal is a human.

In some embodiments, the neurodegenerative disease is Alzheimer's disease. In some embodiments, the axonal spheroid is a plaque-associated axonal spheroid that is associated with an amyloid plaque found in an Alzheimer's disease patient.

In some embodiments, the compound that reverses, ameliorates, and/or prevents a formation or enlargement of an axonal spheroid in a neuron of the subject is compound that:
downregulates an activity and/or expression level of phospholipase D3 (PLD3) in a neuron affected by the neurodegenerative condition;
upregulates an activity and/or expression level of transcription factor EB (TFEB) in a neuron affected by the neurodegenerative condition; and/or
upregulates an activity and/or expression level of autophagy-related protein 5 (ATG5) in a neuron affected by the neurodegenerative condition.

What is considered as “effective amount” by the specification is described elsewhere herein.

In some embodiments, the compound that downregulates the expression level and/or activity of PLD3 includes a small molecule inhibitor of PLD3, a protein inhibitor of PLD3, or a compound that downregulates the expression level or the activity of PLD3 by RNA interference, by ribozyme, by CRISPR knockout/knockdown, or by producing a trans-dominant negative mutant, and so forth.

In some embodiments, the compound that downregulates the expression level or the activity of PLD3 acts at the genomic level. For example, the expression level of PLD3 can be down-regulated by gene knockout, such as CRISPR knockout and other knockout techniques.

In some embodiments, the compound that downregulates the expression level or the activity of PLD3 acts at the transcriptional level or the translational level. For example, the expression level of PLD3 can be down-regulated by gene knockdown, such as by RNA interference technique, ribozyme knockdown, or CRISPR knockdown.

In some embodiments, the compound that downregulates the expression level or the activity of PLD3 acts at the post-translational level. For example, the expression level of PLD3 can be down-regulated by targeted protein degradation, such as proteolysis-targeting chimera (PROTAC) and other protein degradation strategies. For example, the activity of PLD3 can be down-regulated by small molecules inhibitors of PLD3, antibodies that neutralizes PLD3, and trans-dominant negative mutant of PLD3.

In some embodiments, the compound that downregulates the expression level or the activity of PLD3 includes a small molecule inhibitor of PLD3, a protein inhibitor of PLD3, or a compound that downregulates the expression level and/or activity of PLD3 by RNA interference, by ribozyme, by CRISPR knockout/knockdown, or by producing a trans-dominant negative mutant, and so forth.

In some embodiments, the compound contemplated herein can be delivered by a vector, such as a plasmid or a viral vector. One of ordinary skill in the art would understand that such vectors can be used to deliver compounds in the form of nucleic acids, such as RNA or DNA. Such vectors are described herein below.

### Downregulating PLD3 by Small Molecule Inhibitors

In some embodiments, the compound that downregulates the expression level or the activity of PLD3 includes a small molecule that inhibits the activity of PLD3. As used herein, the term “small molecule” refers to a molecule having a size of less than 2000, 1800, 1600, 1400, 1200, 1000, 800, or 600 daltons.

Since PLD3 is a member of the member of the phospholipase D family, one of ordinary skill in the art would expect that many known phospholipase inhibitors could inhibit the activity of PLD3. Examples of small molecule phospholipase inhibitors includes clofazimine (also known as Lamprene or MNKD 101), RABI-767, MRX-4, MRX-6, and VEN 308.

Further examples of small molecule phospholipase inhibitors include PLD3 inhibitors cited in Shirey, et al., Bioorg. Med. Chem. Lett. 49 (2021): 128293 (incorporated herein in its entirety by reference).

In some embodiments, the small molecule inhibitors of PLD3 include

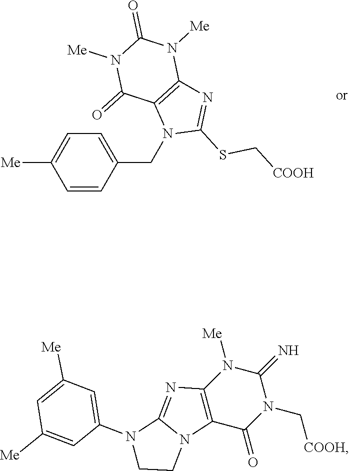 [SMILES: Cc1ccc(Cn2c(SCC(=O)O)nc3c2c(=O)n(C)c(=O)n3C)cc1 ; Cc1cc(C)cc(N2CCn3c2nc2c3c(=O)n(CC(=O)O)c(=N)n2C)c1]

or a salt or solvate thereof.

In some embodiments, the small molecule inhibitor comprises a PROTAC or a Proteolysis Targeting Chimeric Molecule. PROTACs are heterobifunctional nanomolecules that can target any protein for ubiquitination and degradation. In certain embodiments, the PROTAC contemplated in the present invention comprises a group that is recognized by the E3 ubiquitin ligase and a group that is recognized by PLD3. The PROTAC is able to simultaneously bind to the PLD3 and the E3 ligase. Formation of such trimeric complex formation leads to the transfer of ubiquitins to the PLD3, marking it for degradation. PROTAC molecules possess good tissue distribution and the ability to target intracellular proteins, thus can be directly applied to cells or injected into animals without the use of vectors. PROTACS useful within the invention can be prepared using any known compound that binds to and/or recognizes and/or inhibits PLD3, which is linked through a linker to an E3 ubiquitin ligase, such as but not limited to those described in WO 2013/106643, WO 2013/106646, and WO 2019/148055.

### Downregulating PLD3 by Protein Inhibitors of PLD3

In some embodiments, the compound that downregulates the expression level or the activity of PLD3 includes a protein that downregulates the expression level or the activity of PLD3.

Since PLD3 is a member of the phospholipase family, one of ordinary skill in the art would expect that many proteins that are known to downregulate the expression level and/or activity of phospholipase could reduce the level activity of PLD3.

Examples of monoclonal and/or polyclonal antibodies that target PLD3 include SBI-3150, NBP1-59921 (Novus Biologicals, Centennial, CO), HPA012800 (Millipore Sigma, St Louis, MO), PA5-52985, PA5-42640, 17327-1-AP, PA5-104016, PA5-31959 (ThermoFisher, Waltham, MA), LS-C155704, LS-C216828 (LSBio, Seattle, WA), and any humanized derivatives thereof.

Examples of non-antibody proteins that inhibit phospholipases include CB-24 (Crotoxin), uteroglobins such as CG100, CG-201, CG367 and CG459, and VRCTC310 (Crotoxin and Cardiotoxin).

In some embodiments, the protein that downregulates the expression level and/or activity of PLD3 is administered in form of a protein. In some embodiments, the protein that downregulates the expression level and/or activity of PLD3 is administered in form of a nucleic acid that expresses the protein, such as an expression vector. The expression vector is described in the “Vector” section elsewhere in the instant specification.

### Downregulating PLD3 by RNA Interference

In some embodiments, the compound that downregulates the activity or expression level of PLD3 includes a nucleic acid that downregulates the activity and/or expression level of PLD3 by the means of RNA interreference.

In some embodiments, the nucleic acid that downregulates the expression level of PLD3 by the means of RNA interreference includes an isolated nucleic acid. In other embodiments, the modulator is an RNAi molecule (such as but not limited to siRNA and/or shRNA and/or miRNAs) or antisense molecule, which inhibits the expression and/or activity of PLD3. In yet other embodiments, the nucleic acid comprises a promoter/regulatory sequence, such that the nucleic acid is preferably capable of directing expression of the nucleic acid. Thus, the instant specification provides expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In certain embodiments, siRNA is used to decrease the level of PLD3. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391 (19): 306-311; Timmons et al., 1998, Nature 395:854; Montgomery et al., 1998, TIG 14 (7): 255-258; Engelke, Ed., RNA Interference (RNAi) Nuts & Bolts of RNAi Technology, DNA Press, Eagleville, PA (2003); and Hannon, Ed., RNAi A Guide to Gene Silencing, Cold Spring Harbor Laboratory Press, Cold Spring Harbor, NY (2003). Soutschek et al. (2004, Nature 432:173-178) describes a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the instant specification also includes methods of decreasing levels of PLD3 using RNAi technology.

In certain embodiments, the instant specification provides a vector comprising an siRNA or antisense polynucleotide. In other embodiments, the siRNA or antisense polynucleotide inhibits the expression of PLD3. The incorporation of a desired polynucleotide into a vector and the choice of vectors is well-known in the art.

In certain embodiments, the expression vectors described herein encode a short hairpin RNA (shRNA) inhibitor. shRNA inhibitors are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleaves the shRNA to form siRNA.

The siRNA, shRNA, or antisense polynucleotide can be cloned into a number of types of vectors as described elsewhere herein. For expression of the siRNA or antisense polynucleotide, at least one module in each promoter functions to position the start site for RNA synthesis.

In order to assess the expression of the siRNA, shRNA, or antisense polynucleotide, the expression vector to be introduced into a cell can also contain either a selectable marker gene or a reporter gene or both to facilitate identification and selection of expressing cells from the population of cells sought to be transfected or infected using a viral vector. In certain embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

Following the generation of the siRNA polynucleotide, a skilled artisan will understand that the siRNA polynucleotide has certain characteristics that can be modified to improve the siRNA as a therapeutic compound. Therefore, in some embodiments, the siRNA polynucleotide is further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrwal et al., 1987, Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

In some embodiments, the RNA interreference oligonucleotides are specifically designed to increase the cellular uptake of these oligonucleotides. Methods of designing oligonucleotides having desirable cellular uptake are described in, e.g., Geary et al., Adv Drug Deliv Rev 87, 46-51 (2015) and Crooke et al., Nature biotechnology 35, 230-237 (2017).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl-methyl-, thio- and other modified forms of adenine, cytidine, guanine, thymine, and uridine. In some embodiments, the oligonucleotides exist as cholesterol conjugated DNA/RNA heteroduplex oligonucleotides (HDOs) such that the oligonucleotides are blood-brain barrier permeable and could reach the central nervous system (CNS) after subcutaneous or intravenous administration (Nagata et al., Nature biotechnology (2021)).

In certain embodiments, an antisense nucleic acid sequence expressed by a plasmid vector is used to inhibit PLD3 protein expression. The antisense expressing vector is used to transfect a mammalian cell or the mammal itself, thereby causing reduced endogenous expression of PLD3.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the instant specification may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the instant specification include oligonucleotide derivatives known in the art which have improved biological activity compared to unmodified oligonucleotides (see U.S. Pat. No. 5,023,243).

### Downregulating PLD3 by Ribozyme

In some embodiments, the compound that down regulates the activity or expression level of PLD3 includes a ribosome that inhibits PLD3 protein expression.

A ribozyme is used to inhibit PLD3 protein expression. Ribozymes useful for inhibiting the expression of a target molecule may be designed by incorporating target sequences into the basic ribozyme structure which are complementary, for example, to the mRNA sequence encoding PLD3. Ribozymes are antisense RNAs which have a catalytic site capable of specifically cleaving complementary RNAs. Therefore, ribozymes having sequence complementary to PLD3 mRNA sequences are capable of downregulating the expression of PLD3 by reduces the level of PLD3 mRNA. Ribozymes targeting PLD3 may be synthesized using commercially available reagents (Applied Biosystems, Inc., Foster City, CA) or they may be genetically expressed from DNA encoding them. In some embodiments, the DNA encoding the ribozymes are incorporated in a vector, which is described in the “Vector” section elsewhere in the instant specification.

### Downregulating PLD3 by CRISPR Knockout/Knockdown and Other Knockouts/Knockdown Techniques

In some embodiments, the compound that down regulates the activity or expression level of PLD3 comprises a nucleic acid that down regulates the expression level of PLD3 by the means of CRISPR knockout.

In some embodiments, the compound down regulates the activity or expression level of PLD3 comprises a CRISPR/Cas9 system for knocking out PLD3.

The CRISPR/Cas9 system is a facile and efficient system for inducing targeted genetic alterations. Target recognition by the Cas9 protein requires a “seed” sequence within the guide RNA (gRNA) and a conserved di-nucleotide containing protospacer adjacent motif (PAM) sequence upstream of the gRNA-binding region. The CRISPR/Cas9 system can thereby be engineered to cleave virtually any DNA sequence by redesigning the gRNA in cell lines (such as 293T cells), primary cells, and CAR T cells. The CRISPR/Cas9 system can simultaneously target multiple genomic loci by co-expressing a single Cas9 protein with two or more gRNAs, making this system uniquely suited for multiple gene editing or synergistic activation of target genes.

The Cas9 protein and guide RNA form a complex that identifies and cleaves target sequences. Cas9 is comprised of six domains: REC I, REC II, Bridge Helix, PAM interacting, HINH, and RuvC. The Recl domain binds the guide RNA, while the Bridge helix binds to target DNA. The HNH and RuvC domains are nuclease domains. Guide RNA is engineered to have a 5′ end that is complementary to the target DNA sequence. Upon binding of the guide RNA to the Cas9 protein, a conformational change occurs activating the protein. Once activated, Cas9 searches for target DNA by binding to sequences that match its protospacer adjacent motif (PAM) sequence. A PAM is a two or three nucleotide base sequence within one nucleotide downstream of the region complementary to the guide RNA. In one non-limiting example, the PAM sequence is 5′-NGG-3′. When the Cas9 protein finds its target sequence with the appropriate PAM, it melts the bases upstream of the PAM and pairs them with the complementary region on the guide RNA. Then the RuvC and HNH nuclease domains cut the target DNA after the third nucleotide base upstream of the PAM.

One non-limiting example of a CRISPR/Cas system used to inhibit gene expression, CRISPRi, is described in U.S. Patent Appl. Publ. No. US2014/0068797. CRISPRi induces permanent gene disruption that utilizes the RNA-guided Cas9 endonuclease to introduce DNA double stranded breaks which trigger error-prone repair pathways to result in frame shift mutations. A catalytically dead Cas9 lacks endonuclease activity. When coexpressed with a guide RNA, a DNA recognition complex is generated that specifically interferes with transcriptional elongation, RNA polymerase binding, or transcription factor binding. This CRISPRi system efficiently represses expression of targeted genes.

CRISPR/Cas gene disruption occurs when a guide nucleic acid sequence specific for a target gene and a Cas endonuclease are introduced into a cell and form a complex that enables the Cas endonuclease to introduce a double strand break at the target gene. In certain embodiments, the CRISPR/Cas system comprises an expression vector, such as, but not limited to, an pAd5F35-CRISPR vector. In other embodiments, the Cas expression vector induces expression of Cas9 endonuclease. Other endonucleases may also be used, including but not limited to, T7, Cas3, Cas8a, Cas8b, Cas10d, Csel, Csyl, Csn2, Cas4, Cas10, Csm2, Cmr5, Fok1, other nucleases known in the art, and any combinations thereof.

In certain embodiments, inducing the Cas expression vector comprises exposing the cell to an agent that activates an inducible promoter in the Cas expression vector. In such embodiments, the Cas expression vector includes an inducible promoter, such as one that is inducible by exposure to an antibiotic (e.g., by tetracycline or a derivative of tetracycline, for example doxycycline). However, it should be appreciated that other inducible promoters can be used. The inducing agent can be a selective condition (e.g., exposure to an agent, for example an antibiotic) that results in induction of the inducible promoter. This results in expression of the Cas expression vector.

In certain embodiments, guide RNA(s) and Cas9 can be delivered to a cell as a ribonucleoprotein (RNP) complex. RNPs are comprised of purified Cas9 protein complexed with gRNA and are well known in the art to be efficiently delivered to multiple types of cells, including but not limited to neurons, stem cells and immune cells (Addgene, Cambridge, MA, Mirus Bio LLC, Madison, WI).

The guide RNA is specific for a genomic region of interest and targets that region for Cas endonuclease-induced double strand breaks. The target sequence of the guide RNA sequence may be within a loci of a gene or within a non-coding region of the genome. In certain embodiments, the guide nucleic acid sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length.

Guide RNA (gRNA), also referred to as “short guide RNA” or “sgRNA”, provides both targeting specificity and scaffolding/binding ability for the Cas9 nuclease. The gRNA can be a synthetic RNA composed of a targeting sequence and scaffold sequence derived from endogenous bacterial crRNA and tracrRNA. gRNA is used to target Cas9 to a specific genomic locus in genome engineering experiments. Guide RNAs can be designed using standard tools well known in the art.

In the context of formation of a CRISPR complex, “target sequence” refers to a sequence to which a guide sequence is designed to have some complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In certain embodiments, a target sequence is located in the nucleus or cytoplasm of a cell. In other embodiments, the target sequence may be within an organelle of a eukaryotic cell, for example, mitochondrion or nucleus. Typically, in the context of an endogenous CRISPR system, formation of a CRISPR complex (comprising a guide sequence hybridized to a target sequence and complexed with one or more Cas proteins) results in cleavage of one or both strands in or near (e.g., within about 1, 2, 3, 4, 5, 6, 7, 8, 9, 10, 20, 50 or more base pairs) the target sequence. As with the target sequence, it is believed that complete complementarity is not needed, provided this is sufficient to be functional.

In certain embodiments, one or more vectors driving expression of one or more elements of a CRISPR system are introduced into a host cell, such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In certain embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g., each in a different intron, two or more in at least one intron, or all in a single intron).

In certain embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Additional domains that may form part of a fusion protein comprising a CRISPR enzyme are described in U.S. Patent Appl. Publ. No. US20110059502, incorporated herein by reference. In certain embodiments, a tagged CRISPR enzyme is used to identify the location of a target sequence.

Conventional viral and non-viral based gene transfer methods can be used to introduce nucleic acids in mammalian and non-mammalian cells or target tissues. Such methods can be used to administer nucleic acids encoding components of a CRISPR system to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell (Anderson, 1992, Science 256:808-813; and Yu, et al., 1994, Gene Therapy 1:13-26).

In certain embodiments, the CRISPR/Cas is derived from a type II CRISPR/Cas system. In other embodiments, the CRISPR/Cas system is derived from a Cas9 protein. The Cas9 protein can be from Streptococcus pyogenes, Streptococcus thermophilus, or other species.

In general, Cas proteins comprise at least one RNA recognition and/or RNA binding domain. RNA recognition and/or RNA binding domains interact with the guiding RNA. Cas proteins can also comprise nuclease domains (i.e., DNase or RNase domains), DNA binding domains, helicase domains, RNAse domains, protein-protein interaction domains, dimerization domains, as well as other domains. The Cas proteins can be modified to increase nucleic acid binding affinity and/or specificity, alter an enzymatic activity, and/or change another property of the protein. In certain embodiments, the Cas-like protein of the fusion protein can be derived from a wild type Cas9 protein or fragment thereof. In other embodiments, the Cas can be derived from modified Cas9 protein. For example, the amino acid sequence of the Cas9 protein can be modified to alter one or more properties (e.g., nuclease activity, affinity, stability, and so forth) of the protein. Alternatively, domains of the Cas9 protein not involved in RNA-guided cleavage can be eliminated from the protein such that the modified Cas9 protein is smaller than the wild type Cas9 protein. In general, a Cas9 protein comprises at least two nuclease (i.e., DNase) domains. For example, a Cas9 protein can comprise a RuvC-like nuclease domain and a HNH-like nuclease domain. The RuvC and HNH domains work together to cut single strands to make a double-stranded break in DNA. (Jinek, et al., 2012, Science, 337:816-821). In certain embodiments, the Cas9-derived protein can be modified to contain only one functional nuclease domain (either a RuvC-like or a HNH-like nuclease domain). For example, the Cas9-derived protein can be modified such that one of the nuclease domains is deleted or mutated such that it is no longer functional (i.e., the nuclease activity is absent). In some embodiments in which one of the nuclease domains is inactive, the Cas9-derived protein is able to introduce a nick into a double-stranded nucleic acid (such protein is termed a “nickase”), but not cleave the double-stranded DNA. In any of the above-described embodiments, any or all of the nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art.

In one non-limiting embodiment, a vector drives the expression of the CRISPR system. The art is replete with suitable vectors that are useful in the instant specification. The vectors to be used are suitable for replication and, optionally, integration in eukaryotic cells. Typical vectors contain transcription and translation terminators, initiation sequences, and promoters useful for regulation of the expression of the desired nucleic acid sequence. The vectors of the instant specification may also be used for nucleic acid standard gene delivery protocols. Methods for gene delivery are known in the art (U.S. Pat. Nos. 5,399,346, 5,580,859 & 5,589,466, incorporated by reference herein in their entireties).

Further, the vector may be provided to a cell in the form of a viral vector. Viral vector technology is well known in the art and is described, for example, in Sambrook et al. (4th Edition, Molecular Cloning: A Laboratory Manual, Cold Spring Harbor Laboratory, New York, 2012), and in other virology and molecular biology manuals. Viruses, which are useful as vectors include, but are not limited to, retroviruses, adenoviruses, adeno-associated viruses, herpes viruses, Sindbis virus, gammaretrovirus and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

In some embodiments, the compound that down regulates the activity or expression level of PLD3 comprises a nucleic acid that down regulates the expression level of PLD3 by the means of CRISPR knockdown. CRISPR knockdown includes, but not limited to, CRISPRCas13 knockdown. (See e.g., Mendez-Mancilla et al., Cell Chemical Biology 29, 1-7, 2021 Jul. 27, and Kushawah et al., Dev Cell. 2020 Sep. 28;54 (6): 805-817. The entireties of which are incorporated herein by reference).

In some embodiments, the present invention includes any other methods for effecting gene knockdown and/editing, which allow for deletion and/or inactivation of PLD3 such as but not limited to those described in WO 2018/236840 (which is incorporated herein in its entirety by reference).

Downregulating PLD3 by Inactivating and/or Sequestering

In some embodiments, the compound that downregulates the activity or expression level of PLD3 includes a protein that downregulates the activity of PLD3 by inactivating and/or sequestering PLD3. In some embodiment, the compound includes a nucleic acid that express the protein that downregulates the activity of PLD3 by inactivating and/or sequestering PLD3. In some embodiments, the compound includes an expression vector that express the protein that downregulates the activity of PLD3 by inactivating and/or sequestering PDL3 (see “Vector” section for descriptions on vectors).

In some embodiments, the compound that downregulates the expression level of PLD3 is a trans-dominant negative mutant of PLD3, and/or a nucleic acid or a vector expressing the trans-dominant negative mutant of PLD3.

Upregulating TFEB and/or ATG5

In some embodiments, the compound that upregulates the activity and/or expression level of TFEB and/or ATG5.

Compounds that upregulate the activity of TFEB includes protein kinase C a and 8 (PKCα and PKC δ), both of which activate TFEB via reduced phosphorylation and increased nuclear localization (Li et al., Nat. Cell Biol. 18, 1065-1077 (2016)). Compounds that upregulate the activity of TFEB includes TFEB.

Compounds that upregulate the expression of ATG5 includes ATG5.

In some embodiments, the PKCα, PKC δ, TFEB, and/or ATG5 are introduced in the form of exogenous proteins. In some embodiments, exogenous PKCα, PKC δ, TFEB, and/or ATG5 are introduced in the form of a nucleic acid that expresses PKCα, PKC δ, TFEB, and/or ATG5 proteins. In some embodiments, the exogenous nucleic acid are expression vectors, which are detailed elsewhere in the instant specification.

Although compounds that target aggregated forms of Aβ (such as Aducanumab) alone have not been proven to be effective in treating Alzheimer's disease due to that these compounds cannot reverse the downstream pathological changes such as the already formed and enlarged axonal spheroids, these compounds are nonetheless expected to be effective when combining with the compounds that reverses or prevents the formation or enlargement of axonal spheroids described herein.

Therefore, in some embodiments, the method of treating, ameliorating, and/or preventing the neurodegenerative condition further includes administering to the subject an effective amount of a compound that removes a protein aggregate in the brain of the subject.

### Method of Reversing or Preventing Formation or Enlargement of Axonal Spheroids

The formation of large axonal enlargements (i.e., the spheroids) is a neurological condition commonly found in many types of neurodegenerative conditions other than Alzheimer's disease. For example, Lou Gehrig's disease (ALS), Huntington's disease, post traumatic encephalopathy, lysosomal storage disorders including Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, and Lewy Body dementia are all known to involve axonal spheroids.

As detailed elsewhere in the instant specification, the present study discovered that the formation and enlargement of axonal spheroids disrupt the propagation of action potentials (APs) along the axons. Using Alzheimer's disease as a model, the present study discovered that axonal spheroids associated with amyloid plaques (also referred to as “plaque-associated axonal spheroids” or “PAAS” herein) disrupt the propagation of action potentials by acting as electric current sinks. The present study further provides methods of reducing the sizes of the axonal spheroids, and found that doing so restores the axonal conduction properties of the axons affected by the axonal spheroids.

The present study demonstrated various methods to reverse or prevent the formation or enlargement of the axonal spheroids in the neurons, including: inhibiting the accumulation of multivesicular bodies (MVBs) in the axon, such as by down-regulating PLD3; increasing the autophagic activity and the lysosomal activity, such as by upregulating transcription factor EB (TFEB); and increasing the autophagic activity, such as by upregulating autophagy-related protein 5 (ATG5).

The present study further demonstrated that reducing the sizes of the axonal spheroids restores the axonal conduction properties of the axons affected by the axonal spheroids.

Therefore, in some embodiments, the instant specification is directed to a method of reversing, ameliorating or preventing a formation or enlargement of an axonal spheroid. In some embodiments, the axonal spheroid blocks or delays a propagation of an action potential (AP) along an axon of a neuron. In some embodiments, the formation or enlargement of axonal spheroids is associated with a neurodegenerative condition. In some embodiments, the neurodegenerative condition is Alzheimer's disease, Lou Gehrig's disease (ALS), Huntington's disease, post traumatic encephalopathy, lysosomal storage disorders including Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, Lewy Body dementia, or combinations thereof. In some embodiments, the neurodegenerative condition comprises Alzheimer's disease. In some embodiments, the axonal spheroids are axonal spheroids associated with amyloid plaques.

In some embodiments, the method of reversing, ameliorating or preventing a formation or enlargement of an axonal spheroid includes contacting a neuron affected by the axonal spheroid or a surround thereof a compound that reverses, ameliorates, and/or prevents a formation or enlargement of an axonal spheroid.

In some embodiments, the compound that reverses, ameliorates, and/or prevents a formation or enlargement of an axonal spheroid in a neuron of the subject is compound that:
downregulates an activity and/or expression level of phospholipase D3 (PLD3) in a neuron;
upregulates an activity and/or expression level of transcription factor EB (TFEB) in a neuron; and/or
upregulates an activity and/or expression level of autophagy-related protein 5 (ATG5) in a neuron.

In some embodiments, the compound that downregulates the activity and/or expression level of PLD3, as well as the compounds that upregulates the activity and/or expression level of TFEB and/or ATG5 are the same as or similar to those as detailed above in the “Method of Treating, Ameliorating, and/or Preventing Neurodegenerative Condition” section.

### Composition for Treating Neurodegenerative Condition

The formation of large axonal enlargements (i.e., the spheroids) is a neurological condition commonly found in many types of neurodegenerative conditions other than Alzheimer's disease. For example, Lou Gehrig's disease (ALS), Huntington's disease, post traumatic encephalopathy, lysosomal storage disorders including Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, and Lewy Body dementia are all known to involve axonal spheroids.

As detailed elsewhere in the instant specification, the present study demonstrated that the formation and enlargement of axonal spheroids disrupt the propagation of action potentials (APs) along the axons. Using Alzheimer's disease as a model, the present study demonstrated that axonal spheroids associated with amyloid plaques (also referred to as “plaque-associated axonal spheroids” or “PAAS” herein) disrupt the propagation of action potentials by acting as electric current sinks. The present study further demonstrated methods of reducing the sizes of the axonal spheroids, and that doing so restores the axonal conduction properties of the axons affected by the axonal spheroids.

The present study demonstrated various methods to reverse or prevent the formation or enlargement of the axonal spheroids in the neurons, including: inhibiting the accumulation of multivesicular bodies (MVBs) in the axon, such as by down-regulating PLD3; increasing the autophagic activity and the lysosomal activity, such as by upregulating transcription factor EB (TFEB); and increasing the autophagic activity, such as by upregulating autophagy-related protein 5 (ATG5).

The present study further demonstrated that reducing the sizes of the axonal spheroids restores the axonal conduction properties of the axons affected by the axonal spheroids.

Therefore, in some embodiments, the instant specification is directed to a composition for treating a neurodegenerative condition in a subject. In some embodiments, the composition includes:
a compound that
   downregulates an activity and/or expression level of phospholipase D3 (PLD3) in a neuron;
   upregulates an activity and/or expression level of transcription factor EB (TFEB) in a neuron; and/or
   upregulates an activity and/or expression level of autophagy-related protein 5 (ATG5) in a neuron; and
at least one pharmaceutically acceptable carrier.

In some embodiments, the compound that downregulates the activity and/or expression level of PLD3, as well as the compounds that upregulates the activity and/or expression level of TFEB and/or ATG5 are the same as or similar to those as detailed above in the “Method of Treating, Ameliorating, and/or Preventing Neurodegenerative Condition” section.

### Vectors

Vectors can increase the stability of the nucleic acids, make the delivery easier, or allow the expression of the nucleic acids or protein products thereof in the cells.

Therefore, in some embodiments, the protein inhibitors or the nucleic acids that that modulates the activity or expression level of PLD3, TFEB and/or ATG5 is incorporated into a vector.

In some embodiments, the instant specification relates to a vector, including the nucleic acid sequence of the instant specification or the construct of the instant specification. The choice of the vector will depend on the host cell in which it is to be subsequently introduced. In certain embodiments, the vector of the instant specification is an expression vector. Suitable host cells include a wide variety of prokaryotic and eukaryotic host cells. In certain embodiments, the expression vector is selected from the group consisting of a viral vector, a bacterial vector and a mammalian cell vector. Prokaryote-and/or eukaryote-vector based systems can be employed for use with the instant specification to produce polynucleotide, or their cognate polypeptides. Many such systems are commercially and widely available.

In some embodiments, the vector is a viral vector. Viral vector technology is well known in the art and is described, for example, in virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

In some embodiments, the viral vector is a suitable adeno-associated virus (AAV), such as the AAV1-AAV8 family of adeno-associated viruses. In some embodiments, the viral vector is a viral vector that can infect a human. The desired nucleic acid sequence, such as the nucleic acids that modulate PLD3, TFEB and/or ATG5 described above, can be inserted between the inverted terminal repeats (ITRs) in the AAV. In various embodiments, the viral vector is an AAV2 or an AAV8. The promoter can be a thyroxine binding globulin (TBG) promoter. In various embodiments, the promoter is a human promoter sequence that enables the desired nucleic acid expression in the brain. In some embodiments, the promoter is a neuron-selective promoter or a neuron-specific promoter. The AAV can be a recombinant AAV, in which the capsid comes from one AAV serotype and the ITRs come from another AAV serotype. In various embodiments, the AAV capsid is selected from the group consisting of a AAV1, AAV2, AAV3, AAV4, AAV5, AAV6, AAV7, and a AAV8 capsid. In various embodiments, the ITR in the AAV is at least one ITR selected from the group consisting of a AAV1, AAV2, AAV3, AAV4, AAV5, AAV6, AAV7, and an AAV8 ITR. In various embodiments, the instant specification contemplates an AAV8 viral vector (recombinant or non-recombinant) containing a desired nucleic acid expression sequence and at least one promoter sequence that, when administered to a subject, causes elevated systemic expression of the desired nucleic acid. In some embodiments, the viral vector is a recombinant or non-recombinant AAV2 or AAV5 containing any of the desired nucleic acid expression sequences described herein. In some embodiments, the AAV is an engineered AAVs for delivering nucleic acid across the blood brain barrier to the central and peripheral nervous systems, such as those as described by Chan et al., Nat Neurosci. 2017 August; 20 (8): 1172-1179. The entirety of this reference is incorporated herein by reference.

In some embodiments, the vector in which the nucleic acid sequence is introduced is a plasmid that is or is not integrated in the genome of a host cell when it is introduced in the cell. Illustrative, non-limiting examples of vectors in which the nucleotide sequence of the instant specification or the gene construct of the instant specification can be inserted include a tet-on inducible vector for expression in eukaryote cells.

The vector may be obtained by conventional methods known by persons skilled in the art (Sambrook et al., 2012). In certain embodiments, the vector is a vector useful for transforming animal cells.

In certain embodiments, the recombinant expression vectors may also contain nucleic acid molecules which encode a peptide or peptidomimetic inhibitor of the instant specification, described elsewhere herein.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

It will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression. The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous.

The recombinant expression vectors may also contain a selectable marker gene which facilitates the selection of transformed or transfected host cells. Suitable selectable marker genes are genes encoding proteins such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest.

### Combination Therapies

In some embodiments, the method of treating, ameliorating, and/or preventing the neurodegenerative condition or the method of reversing or preventing formation and/or enlargement of axonal spheroids includes administering to the subject the effective amount of at least one compound and/or composition contemplated within the disclosure.

In some embodiments, the composition for treating neurodegenerative condition includes at least one compound and/or composition contemplated within the disclosure.

In some embodiments, the subject is further administered at least one additional agent that treats, ameliorates, and/or prevents a disease and/or disorder contemplated herein. In other embodiments, the compound and the at least one additional agent are co-administered to the subject. In yet other embodiments, the compound and the at least one additional agent are co-formulated.

The compounds contemplated within the disclosure are intended to be useful in combination with one or more additional compounds. These additional compounds may comprise compounds of the present disclosure and/or at least one additional agent for treating neurodegenerative conditions, and/or at least one additional agent that treats one or more diseases or disorders contemplated herein.

A synergistic effect may be calculated, for example, using suitable methods such as, for example, the Sigmoid-Emax equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations contemplated within the disclosure may be administered to the subject either prior to or after the onset of a disease and/or disorder contemplated herein. Further, several divided dosages, as well as staggered dosages may be administered daily or sequentially, or the dose may be continuously infused, or may be a bolus injection. Further, the dosages of the therapeutic formulations contemplated within the disclosure may be proportionally increased or decreased as indicated by the exigencies of the therapeutic or prophylactic situation.

Administration of the compositions contemplated within the disclosure to a patient, preferably a mammal, more preferably a human, may be carried out using known procedures, at dosages and for periods of time effective to treat a disease and/or disorder contemplated herein in the patient. An effective amount of the therapeutic compound necessary to achieve a therapeutic effect may vary according to factors such as the state of the disease or disorder in the patient; the age, sex, and weight of the patient; and the ability of the therapeutic compound contemplated within the disclosure to treat a disease and/or disorder contemplated herein in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound contemplated within the disclosure is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions contemplated within the disclosure may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds contemplated within the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms contemplated within the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease and/or disorder contemplated herein.

In certain embodiments, the compositions of the disclosure are formulated using one or more pharmaceutically acceptable excipients or carriers. In certain embodiments, the pharmaceutical compositions of the disclosure comprise a therapeutically effective amount of a compound of the disclosure and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions of the disclosure are administered to the patient in dosages that range from one to five times per day or more. In another embodiment, the compositions of the disclosure are administered to the patient in range of dosages that include, but are not limited to, once every day, every two, days, every three days to once a week, and once every two weeks. It is readily apparent to one skilled in the art that the frequency of administration of the various combination compositions of the disclosure varies from individual to individual depending on many factors including, but not limited to, age, disease or disorder to be treated, gender, overall health, and other factors. Thus, the disclosure should not be construed to be limited to any particular dosage regime and the precise dosage and composition to be administered to any patient is determined by the attending physical taking all other factors about the patient into account.

Compounds of the disclosure for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the disclosure is from about 1 mg and about 2,500 mg. In some embodiments, a dose of a compound of the disclosure used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of neurodegenerative conditions in a patient.

Formulations may be employed in admixtures with conventional excipients, i.e., pharmaceutically acceptable organic or inorganic carrier substances suitable for intracranially, intrathecal, oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the disclosure include oral, nasal, rectal, intravaginal, parenteral, buccal, sublingual or topical. The compounds for use in the disclosure may be formulated for administration by any suitable route, such as for oral or parenteral, for example, transdermal, transmucosal (e.g., sublingual, lingual, (trans) buccal, (trans) urethral, vaginal (e.g., trans- and perivaginally), (intra) nasal and (trans) rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the disclosure may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY—P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

The present disclosure also includes a multi-layer tablet comprising a layer providing for the delayed release of one or more compounds of the disclosure, and a further layer providing for the immediate release of another medication. Using a wax/pH-sensitive polymer mix, a gastric insoluble composition may be obtained in which the active ingredient is entrapped, ensuring its delayed release.

### Parenteral Administration

For parenteral administration, the compounds of the disclosure may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this disclosure include dosage forms as described in U.S. Pat. Nos. 6,340,475; 6,488,962; 6,451,808; 5,972,389; 5,582,837; and 5,007,790. Additional dosage forms of this disclosure also include dosage forms as described in U.S. patents application Ser. Nos. 20/030,147952; 20030104062; 20030104053; 20030044466; 20030039688; and 20020051820. Additional dosage forms of this disclosure also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the disclosure may be administered in the form of microparticles, for example, by injection or in the form of wafers or discs by implantation.

In certain embodiments of the disclosure, the compounds of the disclosure are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present disclosure depends on the age, sex and weight of the patient, the current medical condition of the patient and the progression of the neurodegenerative condition in the patient being treated. The skilled artisan is able to determine appropriate dosages depending on these and other factors.

A suitable dose of a compound of the present disclosure may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the modulator of the disclosure is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the patient's condition, to a level at which the improved disease is retained. In certain embodiments, patients require intermittent treatment on a long-term basis upon any recurrence of symptoms and/or infection.

The compounds for use in the method of the disclosure may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. Capsid assembly modulators exhibiting high therapeutic indices are preferred. The data obtained from cell culture assays and animal studies are optionally used in formulating a range of dosage for use in human. The dosage of such capsid assembly modulators lies preferably within a range of circulating concentrations that include the ED₅₀ with minimal toxicity. The dosage optionally varies within this range depending upon the dosage form employed and the route of administration utilized.

Those skilled in the art recognizes, or is able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in assay and/or reaction conditions, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

### Delivery

In certain embodiments, the compound contemplated herein (including but not limited to nucleic acids) can be more efficiently delivered to the cell nucleus by coupling the compound with the monoclonal anti-DNA antibody 3E10, which penetrates living cells and localizes in the nucleus without causing any apparent harm to the cell (Hansen J E, et al., Intranuclear protein transduction through a nucleoside salvage pathway. J Biol Chem 2007; 282:20790-3; see also WO 2020/047353 and WO 2021/042060, all of which are incorporated herein in their entireties by reference). 3E10 and its single-chain variable fragment (3E10 scFv) have been developed as an intracellular delivery system for macromolecules. After localizing in the cell nucleus, 3E10 scFv is largely degraded within 4 hours, thus further minimizing any potential toxicity.

In certain embodiments, the compounds contemplated herein (including but not limited to nucleic acids) can be more efficiently delivered to the central nervous system using certain lipid nanoparticle formulations known in the art, such as but not limited to those described in Cullis, P. R. et al., Molecular Therapy Vol. 25 No 7 Jul. 2017. See also US20150165039 and WO 2014/008334, all of which are incorporated herein in their entireties by reference.

In certain embodiments, the compounds contemplated herein can be more efficiently delivered to tissue by coupling with certain protein fragments, called “pHLIP” (pH (Low) Insertion Peptide), which allow for the cargo to accumulate in acidic environments within the body. In certain embodiments, a polypeptide with a predominantly hydrophobic sequence long enough to span a membrane lipid bilayer as a transmembrane helix (TM) and comprising one or more dissociable groups inserts across a membrane spontaneously in a pH-dependent fashion placing one terminus inside cell. The polypeptide conjugated with various functional moieties delivers and accumulates them at cell membrane with low extracellular pH. The functional moiety conjugated with polypeptide terminus placed inside cell are translocated through the cell membrane in cytosol. The peptide and its variants or non-peptide analogs can be used to deliver therapeutic, prophylactic, diagnostic, imaging, gene regulation, cell regulation, or immunologic agents to or inside of cells in vitro or in vivo in tissue at low extracellular pH. See also US20080233107, WO2012/021790, US20120039990, US20120142042, US20150051153, US20150086617, and US20150191508, all of which are incorporated herein in their entireties by reference.

### Brain Delivery

According to some embodiments, downregulating the PLD3 level and/or activity requires downregulating the level and/or activity in the brain of the subject. Similarly, upregulating TFEB and/or ATG5 level and/or activity sometimes requires upregulating the level and/or activity in the brain of the subject, as well. As such, in some embodiments, the compounds or compositions of the present invention (such as small molecules, proteins, or nucleic acids) are delivered to the brain of the subject. One of ordinary skill in the art would understand that various strategies have been developed in the art to achieve such delivery. Non-limiting examples of such brain delivery strategies are described herein.

### Virus-Based Delivery Strategies

Virus-based brain delivery strategies are able to deliver nucleic acid, which encodes for the protein inhibitors, the RNA interference molecules, CRISPR components, etc., to the brain.

For example, Chan et al. (Nat Neurosci. 2017 August;20 (8): 1172-1179) describes an AAV-PHP.eB capsid that is capable of transducing the central nervous systems when administered intravenously. Kumar et al. (Nat Methods. 2020 May;17 (5): 541-550) describes the identification of recombinant adeno-associated virus (rAAV) capsid variants that can transduce the central nervous system broadly, exhibit bias toward vascular cells and astrocytes, target neurons with greater specificity, or cross the blood-brain barrier. Deverman et al. (Nat Biotechnol. 2016 February;34 (2): 204-9) describes AAV variants (such as the AAV-PHP.B variant) that, when intravenously injected, transfers genes throughout the CNS with increased efficiency as compared to AAV9. Haery et al. (Front Neuroanat. 2019; 13:93) provides a general summary of using AAV for targeted neuronal manipulation. Chuapoco et al. (Nat Nanotechnol. 2023 October;18 (10): 1241-1251) describes an engineered AAV variant (AAV.CAP-Mac) that was confirmed to be able to transfer gene throughout the brains in several types of tested non-human primates when injected intravenously. Murlidharan et al. (Mol Ther Nucleic Acids. 2016 Jul. 19;5 (7): e338) describes an AAV chimeric (AAV2g9) which, when injected intracranially, achieved minimal systemic leakage, decreased sequestration and gene transfer in off-target organs.

### Direct CNS Administration

In some embodiments, the small molecules, nucleic acids and proteins contemplated herein are administered directly to the brain. These strategies does not require carriers that are able to cross the blood-brain barrier (BBB) and, for therapeutic compounds that cannot cross the BBB, can avoid the contact of the therapeutic compounds with non-CNS tissues.

For example, Benatti et al. (Mol Ther Methods Clin Dev. 2023 Sep. 26:31:101122) describes the use of intracerebroventricular (i.e.v.) catheters having a 3D-printed anchorage system to repeatedly dose oligonucleotides to the brain of a subject. O'Reilly et al. (Mol Ther. 2023 Jun. 7;31 (6): 1661-1674) and Alterman et al. (Nat Biotechnol. 2019 Aug;37 (8): 884-894) both describe the delivery of di-valent siRNAs to the brain tissues/the central nervous system by cerebral spinal fluid (CSF) infusions. Lonser (J Neurosurg. 2020 Jul. 10;134 (6): 1751-1763) describes the direct convective delivery of AAV gene therapy for the treatment of neurological disorders.

### Nanoparticles

In some embodiments, the small molecules, nucleic acids and proteins contemplated herein are delivered to the brain using nanoparticles as carriers. For example, Sava et al. (J Drug Deliv Sci Technol. 2021 June: 63:102517), Sava et al. (Nanomedicine. 2020 February: 24:102119) and Sanchez-Ramos et al. (J Drug Deliv Sci Technol. 2018 February: 43:453-460) describe the brain delivery of siRNA packaged in chitosan-based nanoparticle following intranasal administration. Lee et al. (BME Front. 2023 Mar. 31:4:0012) describes strategies to attach antibodies to nanoparticles and the targeting of the complex in the central nervous system. Josowitz (Nanotechnology. 2022 Dec. 2;34 (7): 10.1088/1361-6528/ac9683) describes using polymer nanocarriers for the delivery of agents in treating brain tumors, and describes strategies for bypassing the blood-brain-barrier, such as focused ultrasound and convection enhanced delivery. Seo et al. (Biomaterials. 2019 May; 201:87-98.) describes treating tumors in the brain with convection-enhanced delivery (CED) of complexes of oligonucleotides packaged in nanoparticles of cationic poly(amine-co-ester) (PACE) or block copolymer of poly(lactic acid) and hyperbranched polyglycerol (PLA-HPG). Chen et al. (Biomaterials. 2018 September; 178:193-203) describes delivering a radiosensitizer to tumors in the brain by packing the radiosensitizer with PEG-poly(ω-pentadecalactone-co-p-dioxanone) nanoparticles and administering by convection-enhanced delivery (CED). Xie et al. (Med X. 2023; 1 (1): 6) provides general information regarding non-viral approaches for delivery across the blood-brain-barrier, including the use of nanoparticles. Metzger et al. (Biomaterials. 2023 February: 293:121959) describes using PEGylated nanocapsules (NCs) to deliver preassembled Cas9-sgRNA ribonucleoproteins into the brain to perform gene editing in the neurons. Wang et al. (Adv Mater. 2023 February;35 (6): e2208018) describes GSH-responsive silica nanocapsules that, when systemically administered, achieved brain-wide delivery. Zou et al. (Sci Adv. 2022 Apr. 22;8 (16): eabm8011) describes packaging Cas9/sgRNA components in a glutathione-sensitive polymer shell incorporating a dual-action ligand that facilitates blood-brain-barrier penetration, and performing gene editing in tumors in the brain.

### Delivery by Transiently Open the Blood-Brain-Barrier

One of the major difficulties of brain delivery, such as the brain delivery of the compounds and compositions herein, is to delivering across the blood brain barrier. Technologies exist to transiently open the blood-brain-barrier, which simplifies the delivery.

For example, Lao (Proc Natl Acad Sci USA. 2023 Aug. 22; 120 (34): e2302910120) describes a method of using focused ultrasound (FUS) to transiently open the BBB for the transport of intravenously delivered CRISPR/Cas9 machinery to the brain. It has been shown that the disruption of the blood-brain-barrier with FUS can allow molecules as large as 150 kDa to cross.

### Examples

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1: Plaque-Associated Axonal Spheroids Disrupts Axonal Conduction

Plaque-associated axonal spheroids (PAAS) are a significant but understudied pathological hallmark in Alzheimer's disease (AD). In Example 1, the pathophysiological significance of axonal spheroids will be investigated, the cellular and molecular mechanisms regarding their development will be elucidated, and potential therapeutic implications of PAAS will be identified.

### Example 1-1: Axonal Spheroids are Stable but Dynamic Structures Found Abundantly Around Amyloid Plaques

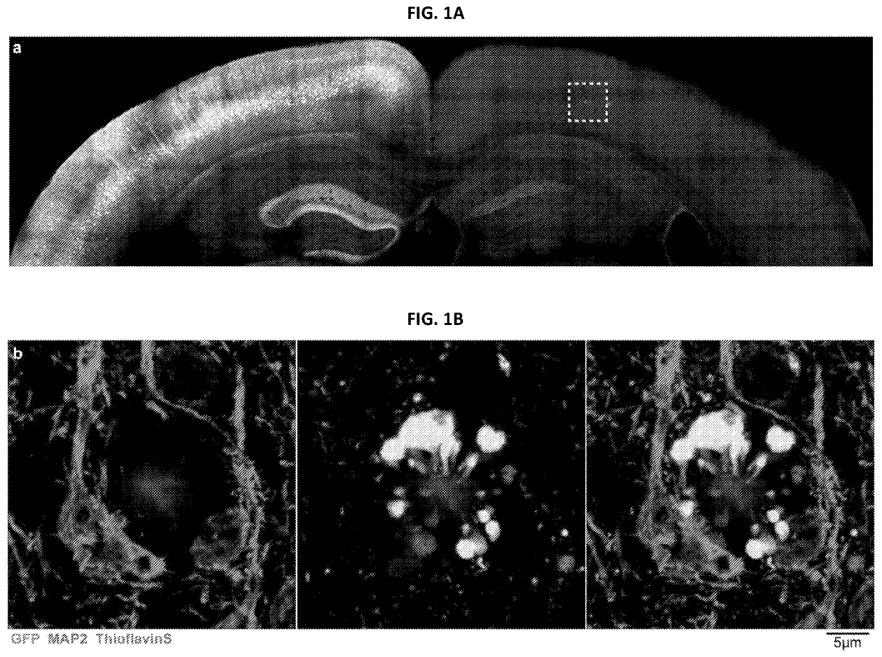

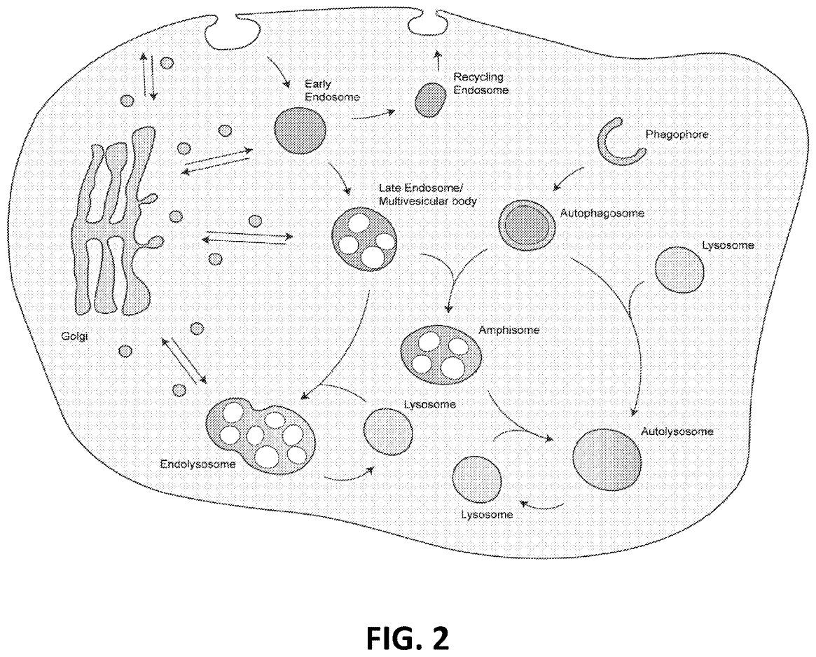

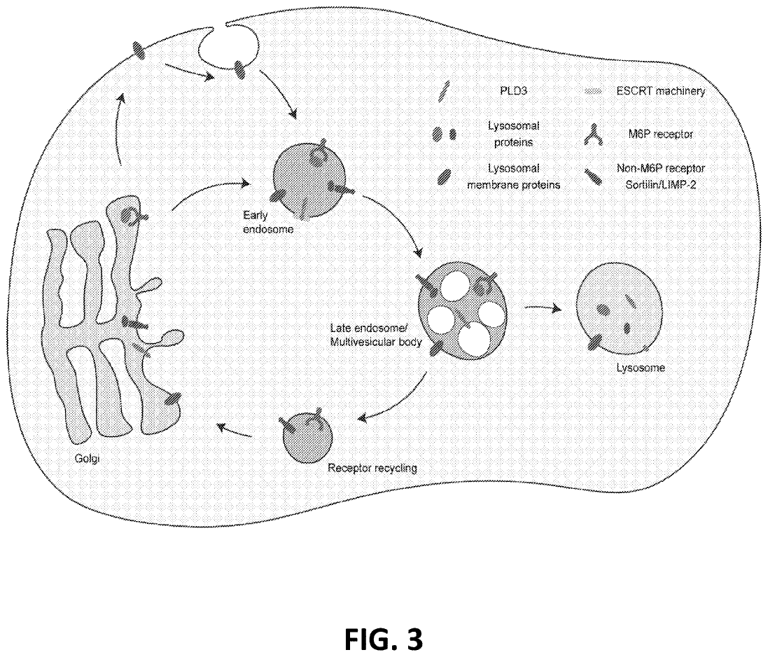

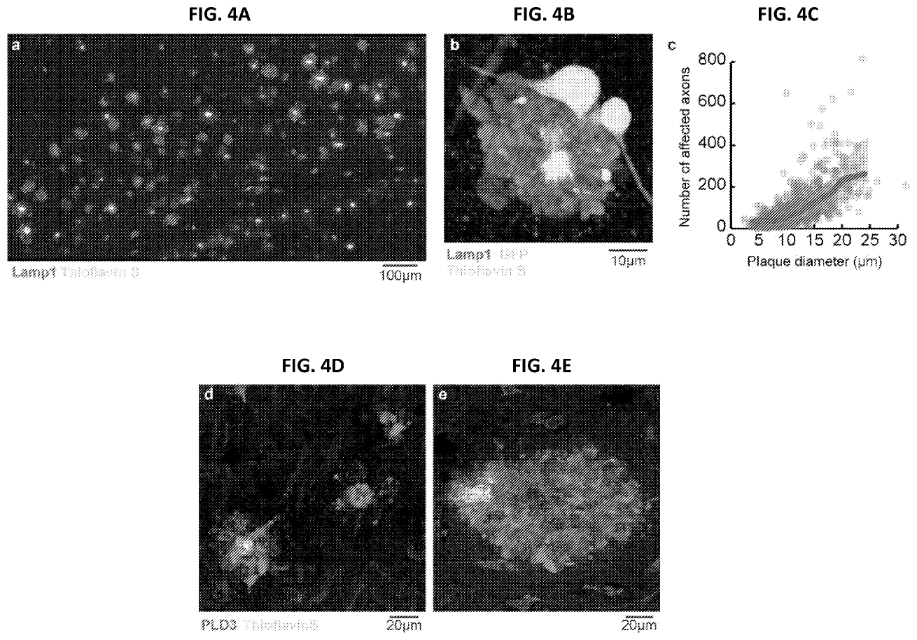

Confocal imaging of axonal spheroids labeled with lysosome-associated membrane protein 1 (LAMP1) or phospholipase D3 (PLD3), two proteins enriched in PAAS, showed that these structures are found abundantly around amyloid plaques in both AD-like mice and human AD patients (FIGS. 4A, 4D and 4E). To estimate the approximate number of axons affected by plaques and thus develop spheroids, a fluorescent reporter-carrying adeno-associated virus (AAV) was injected into one hemisphere of 5×FAD mice. This enabled sparse labeling of transcallosal projections in the contralateral cortex, which allowed detailed imaging and analysis of individual axons at higher resolution (FIGS. 1A and 4B). Based on the average volume of individual PAAS per axon segment using viral-mediated labeling, and the total volume of the PAAS halo around plaques through LAMP1 immunofluorescence (FIG. 4B), it was estimated that individual plaques can on average affect hundreds of axons (FIG. 4C). Given the abundance of amyloid plaques in the AD brain, this indicates that massive numbers of axons can be affected, highlighting the potential significance of PAAS as a mechanism of neural network dysfunction.

In vivo time lapse imaging of virally labeled axons around plaques revealed that PAAS can be very stable over intervals of up to months (FIGS. 5A-5D). While most PAAS increased in size overtime, a substantial number declined in volume or disappeared during this interval, without the loss of the parent axon (FIGS. 5A-5D), consistent with previous reports. This supports the idea that PAAS are not a feature of degenerating axons but are instead stable axonal structures that may affect neuronal circuits for extended intervals, while at the same time having the potential for reversibility.

### Example 1-2: Plaque-Associated Axonal Spheroids Block Action Potential Propagation

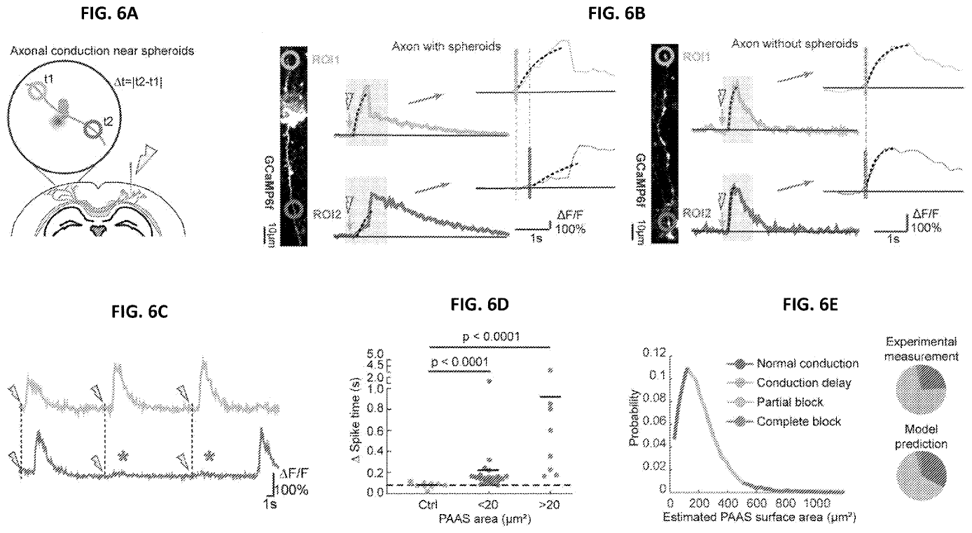

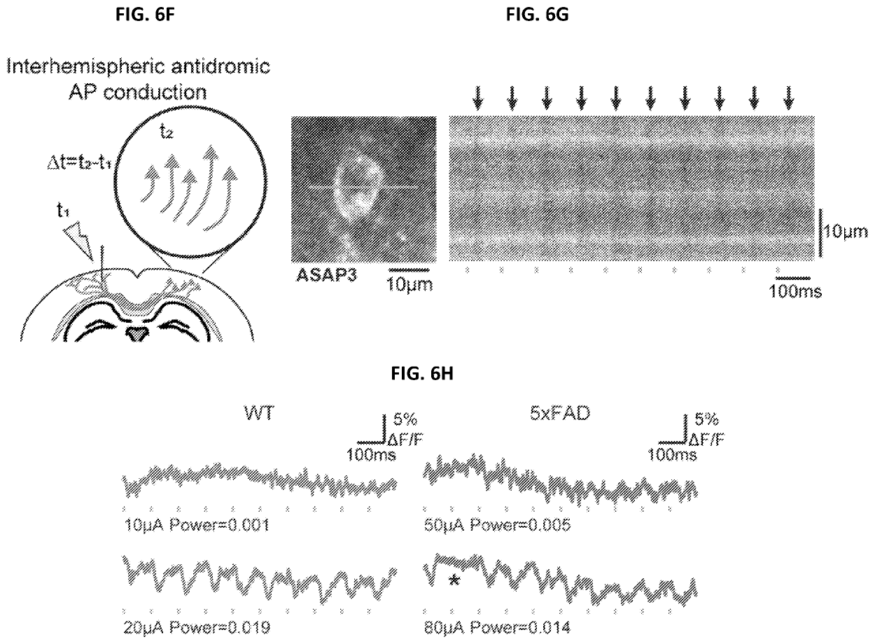

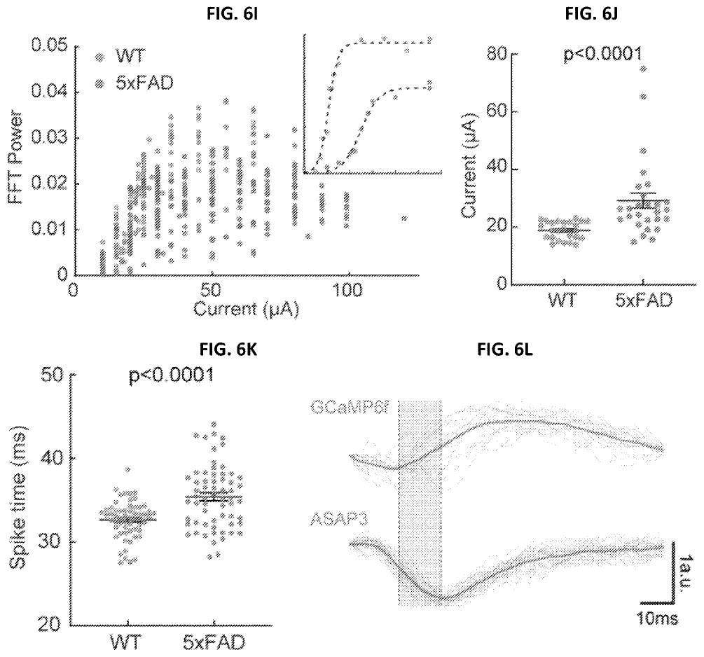

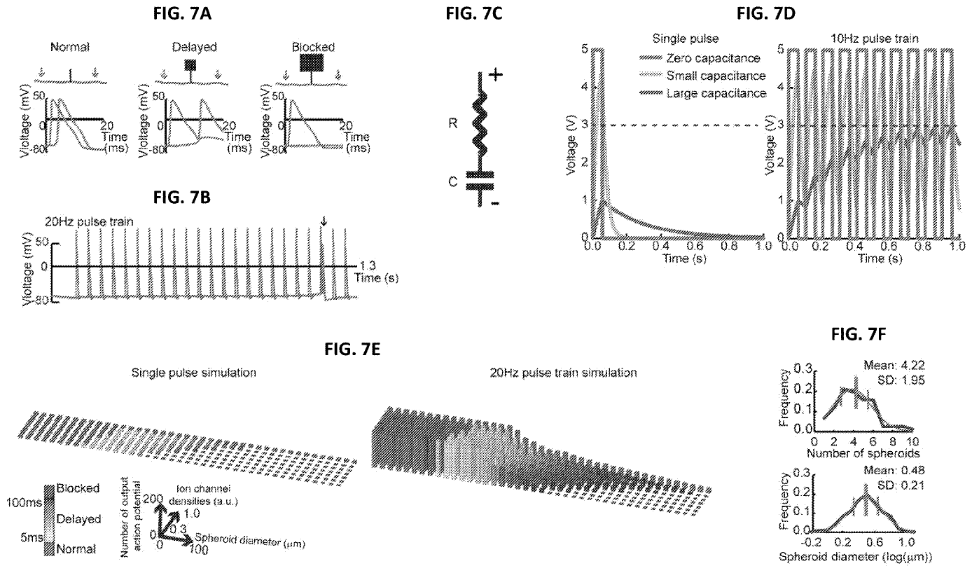

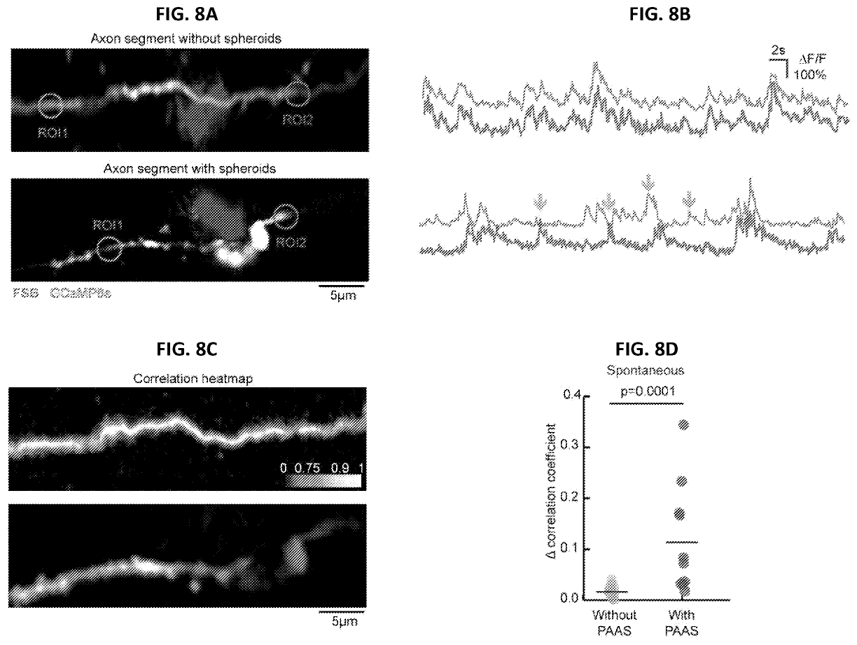

The finding that hundreds of axons around each amyloid plaque develop spheroids which remain stable for extended periods of times indicate that these structures might lead to severe functional consequences. To experimentally examine how PAAS might disrupt neuronal circuits, a strategy for measuring the propagation of action potentials in individual axons through Ca²⁺ imaging in the live mouse brain was developed. The calcium sensor, GCaMP6f, was virally expressed through delivery of adeno-associated viral (AAV) vectors to one hemisphere of the mouse brain (as in FIG. 1A) and performed Ca²⁺ imaging of individual projection axons on the contralateral cortex (FIG. 6A). AP propagation after electrically stimulating the ipsilateral hemisphere was measured with trains of electrical pulses and compared the rise times of Ca²⁺ transients (a surrogate for AP spike time) at two regions of interest (ROI) located on axon segments on both sides of individual PAAS (FIG. 6A). It was found that the onset of the rise-times was consistently delayed over intervals ranging from hundreds of milliseconds to seconds (FIGS. 6β-6D). Given that a series of pulses of electrical stimulation was used to induce trains of AP spikes, it was concluded that the unusually long delays in Ca²⁺ rise-times observed, were due to conduction blocks of a substantial proportion of individual AP spikes, once they reached individual spheroids (FIG. 7B). On the other hand, comparison of rise times at two ROIs in axon segments without spheroids or at ROIs located on the same side of axonal segments adjacent to spheroids (FIG. 6B, right panel) demonstrated no difference in the onset of rise-times, strongly suggesting a deleterious effect of PAAS, rather than broad spheroid-independent defects in axonal conduction. Furthermore, in addition to electrically evoked responses, we also imaged spontaneous Ca²⁺ transients in individual axons and observed similar conduction abnormalities in segments with PAAS (FIG. 8). Importantly, regardless of whether the APs were the result of spontaneous or electrically induced neuronal activity, it was observed that larger spheroids caused more severe conduction blocks (FIGS. 6D and 8D).

To further dissect the electrophysiological basis of conduction block caused by axonal spheroids, how these spheroids affect axonal conduction was computationally modeled. It was found that the likelihood of conduction disruption of action potentials (AP) for a particular axonal segment, markedly increased as a function of the total PAAS surface area (FIGS. 7A-7F). The in silico model predicted that PAAS behave as capacitors that act as electric current sinks for incoming action potentials and can thus cause conduction blocks or prolonged delays (FIG. 7A-7F). The experimental observations were consistent with the computational modeling results, demonstrating that the size of individual PAAS is a critical determinant of the degree of conduction defects associated with spheroids (FIG. 6E).

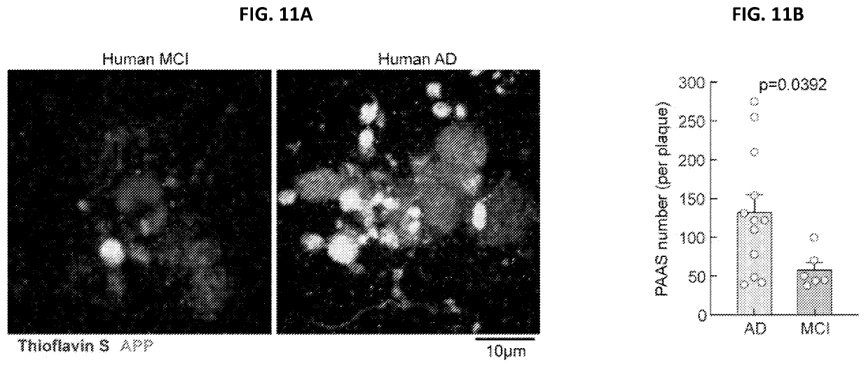

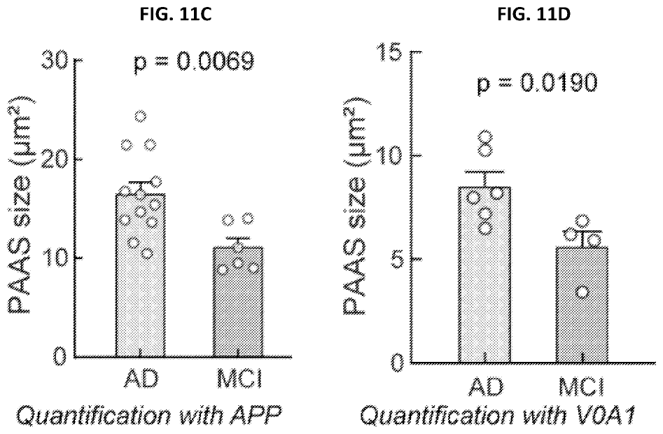

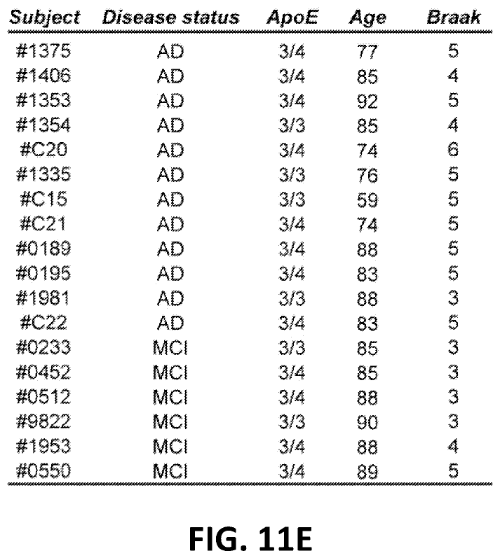

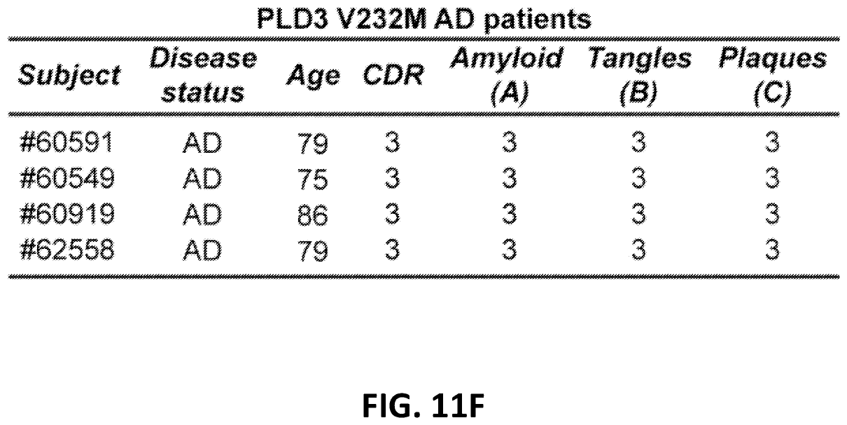

The present study further implemented in vivo voltage imaging using the genetically encoded voltage sensor ASAP325. AAV2-Syn-ASAP3 was intra-cortically injected in the same manner as was done for GCaMP6f (FIG. 6F). Given the relatively low signal-to-noise ratio when imaging with genetically encoded voltage sensors, it was not feasible to perform single-trial experiments of individual axons. Instead, the present study stimulated the axons in one hemisphere and recorded the contralateral hemisphere antidromic action potential, visualized at the cell bodies rather than axons (FIGS. 6F-6G), which markedly improved signal-to-noise ratio. Using this strategy, it was found that in 5×FAD mice, there was a marked increase in the electric current required to induce the interhemispheric propagation of an action potential through single trial stimulations (FIGS. 6H-6J), consistent with PAAS acting as current sinks. Furthermore, the present study also observed frequent delays on AP propagation when comparing 5×FAD and WT mice (FIGS. 6K-6L) in agreement with the Ca²⁺ imaging experiments. Altogether, the imaging data as well as the computational modeling highlight the prevalence of action potential conduction blocks resulting from spheroid pathology in AD. Given the long interhemispheric distances, the probability of axons to encounter amyloid plaques and develop spheroids is relatively high. While the density of amyloid plaques in humans is lower than in mice, the present study hypothesizes that given their much greater axonal lengths in humans, this would increase the probability of adjacency to amyloid plaques, and thus the likelihood of disruption in axonal connectivity. In support of this idea, through quantitative analysis of postmortem brains, the present study found a much greater average number of axonal spheroids per amyloid plaque and greater PAAS size (FIGS. 11A-11E) in individuals with moderate to severe AD, compared to those with mild cognitive impairment (MCI). This suggests that both PAAS number and size could be important factors that determine the degree of neural circuit disruption and cognitive deficits in AD.

### Examples 1-3: Prolonged Ca²⁺ Decay Times in Axon Segments with PAAS

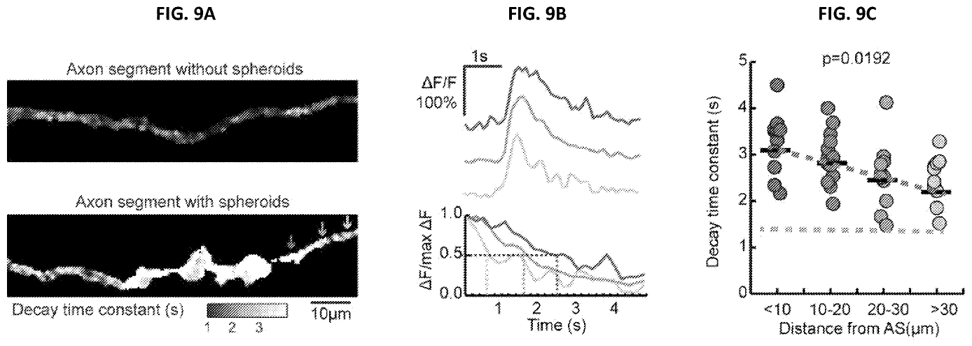

In addition to Ca²⁺ rise times, the present study also calculated Ca²⁺ decay times in axon segments with and without PAAS. It was found that in axons with spheroids, the decay times are markedly longer in axons regions that was closer to PAAS (FIGS. 9A-9C). In contrast, the decay times were identical along an axon without spheroids. This indicates that there is a severe calcium dyshomeostasis in regions of axons with spheroids. Lysosomes are intracellular stores for calcium. Given that LAMP1-positive vesicles accumulate in the spheroids, the calcium dysregulation might be due to lysosome calcium.

### Example 1-4: Plaque-Associated Axonal Spheroids Disrupt Interhemispheric Connectivity

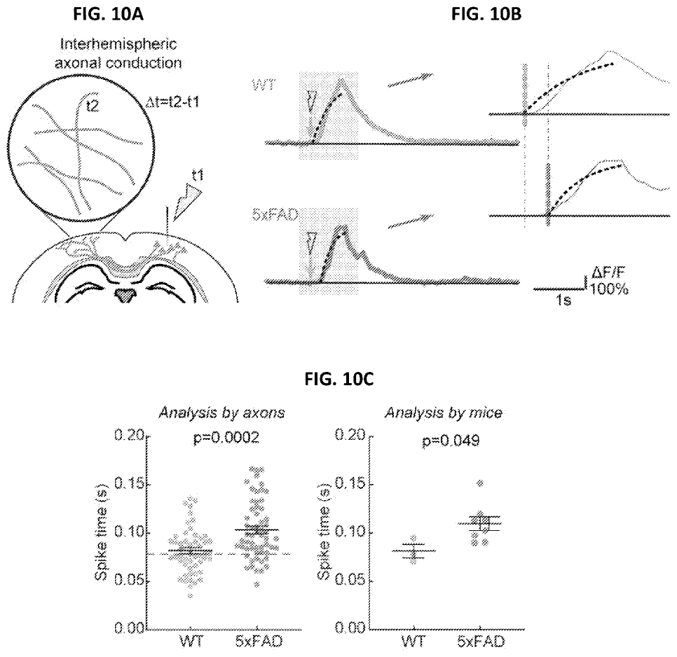

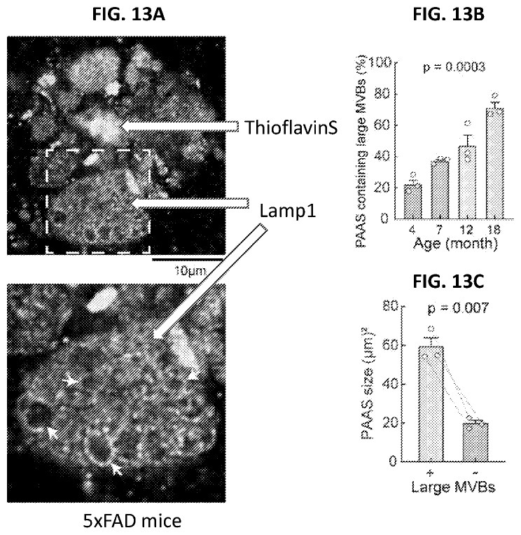

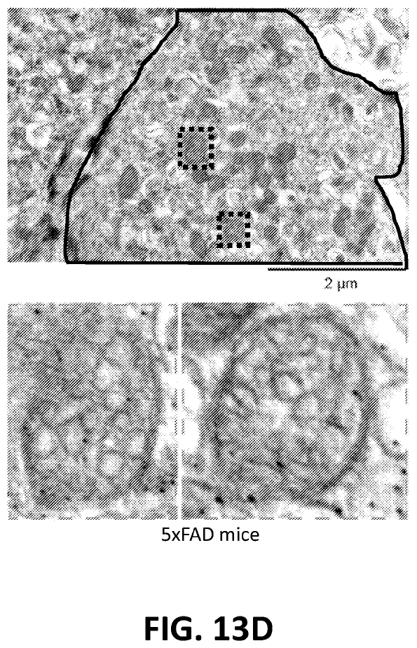

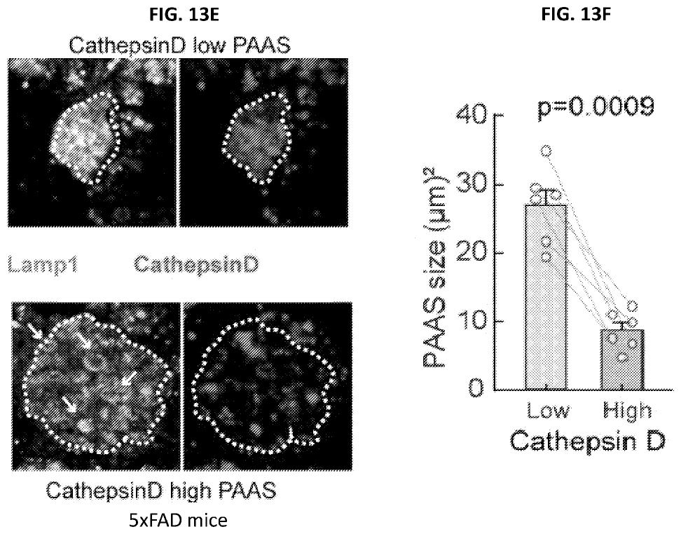

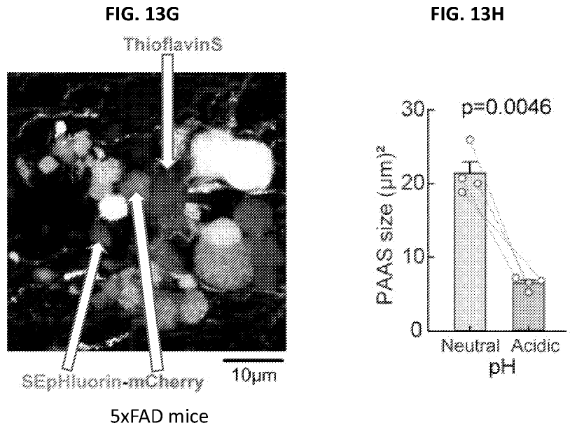

Given that the present study found marked abnormalities in local axonal conduction around plaques, if this was associated with more widespread defects in long-range cortical connectivity was further explored, by measuring interhemispheric conduction velocity through calcium imaging in live 5×FAD mice. To achieve this, AAV9-Syn-GCaMP6f were stereotaxically injected to label a homogeneous population of closely located cortical neurons in somatosensory cortex, which assured comparable axonal distances to the contralateral hemisphere imaging region across different mice. The present study then imaged contralateral projecting axons, while electrically stimulating the ipsilateral GCaMP6f-labeled neurons (FIGS. 10A-10C). It was found that the interhemispheric conduction velocities in wildtype mice were of similar magnitude as those previously reported using slice electrophysiology recordings (Lohmann et al., J Comp Neurol 344, 543-558, (1994) and Telfeian et al., Neurosci Lett 343, 121-124, (2003)). However, in 5×FAD mice, Ca²⁺ rise times in projecting axons were markedly delayed (FIGS. 10B-10C). This indicates that given the long interhemispheric distances, the probability of axons to encounter amyloid plaques and develop spheroids is relatively high. While the density of amyloid plaques in humans is lower than in mice, the axonal lengths are much greater, thereby increasing the probability of adjacency to amyloid plaques, resulting in potential disruption of axonal connectivity. Importantly, through quantitative analysis of human postmortem brains, the present study found a much greater average number of axonal spheroids per amyloid plaque (FIGS. 11A and 11B) and greater PAAS size (FIG. 13E) in individuals with moderate to severe AD, compared to those with mild cognitive impairment (MCI). Altogether, this indicates that both PAAS number and size could be important factors that determine the degree of neural circuit disruption and cognitive deficits in AD.

### Example 1-5

The experiments describe in Example 1 showed that hundreds of axons around each amyloid plaque develop spheroids and rather than being retraction bulbs from degenerating axons, these structures are stable for extended periods of time and therefore could significantly disrupt neural circuits. In vivo single axon calcium imaging showed that when neurons are stimulated with a train of electrical pulses, spheroids cause axonal conduction blocks, which allow only a fraction of AP spikes to propagate, giving the appearance of conduction delays ranging from hundreds of milliseconds to seconds. Importantly, larger PAAS cause more severe conduction blocks, consistent with computational modeling showing that PAAS function as electrical capacitors that act as current sinks, and that PAAS size is a major determinant of the degree of conduction defects. These data indicates that the large number of plaques present in the AD brain have the potential to significantly affect neural networks by widespread disruption of axonal connectivity. Consistent with this view, the quantitative histopathology analysis of human postmortem brain from AD or MCI patients showed that PAAS size and number correlates well with the degree of premortem cognitive decline.

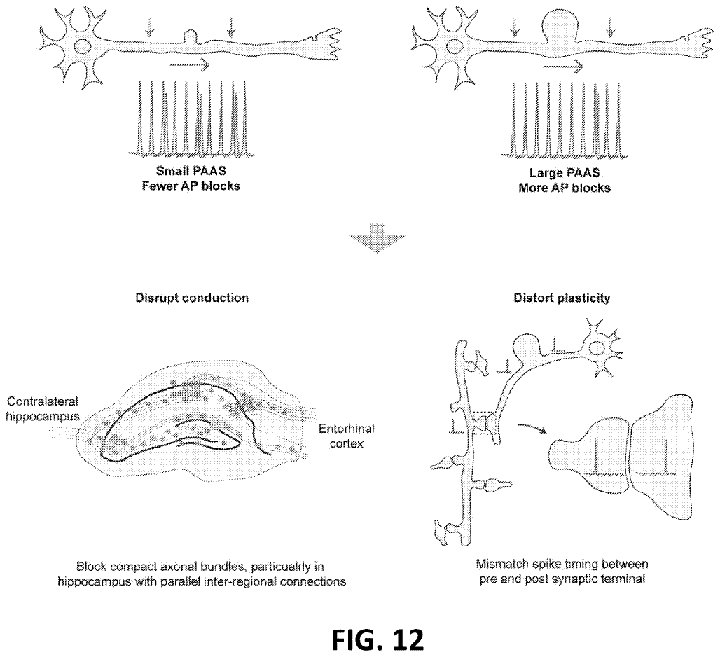

In AD, strategically located amyloid plaques could have deleterious effects in regions of the brain like the hippocampus where parallel compact axonal bundles follow a stereotyped projection path along a tri-synaptic loop (Eichenbaum et al., Cold Spring Harb Symp Quant Biol 61, 197-206 (1996)). A single plaque could lead to the development of numerous spheroids in those compact axon bundles, thus severely disrupt axon conduction both within the tri-synaptic circuit, as well as in inter-regional connections between hippocampus and multiple regions of the cortex. PAAS are particularly detrimental to neural processes that rely on temporally precise long-range coordination among brain regions, such as memory formation. Specifically, during system consolidation, hippocampus replays the representations of individual memorandums in temporally compressed neural spike sequences (Buzsaki et al., Hippocampus 25, 1073-1188, (2015)). And these sequential replays then guide distributed modification of synaptic connections, closely coupled with network oscillations (Khodagholy et al., Science 358, 369-372, (2017) and Ramirez-Villegas et al., Nature 589, 96-102, (2021)). Two aspects of this process may be disrupted by PAAS: First, PAAS-mediated conduction delays or blockades could disrupt the faithful propagation of memory-encoding neural sequences in the brain. Similar to PAAS-mediated conduction disruption, experimentally disrupting the precise phase-lock synchronization of neural activities during system consolidation leads to failure of memory formation (Wu et al., Nat Neurosci 20, 571-580, (2017), Jadhav et al., Science 336, 1454-1458, (2012), and Gridchyn et al., Neuron 106, 291-300 e296, (2020)). Meanwhile, PAAS could further interfere with the synaptic weight modification process, since precise timing of firing in axonal terminals and postsynaptic cells provides pivotal guidance of synaptic plasticity (Billings et al., J Neurophysiol 101, 2775-2788, (2009) and Dan et al., Neuron 44, 23-30, (2004)). Together, axonal conduction delay and block caused by PAAS may distort the neural processes underlying memory formation, potentially contributing to the anterograde amnesia in AD. Furthermore, axonal conduction defects could disrupt long-range connectivity between brain networks, as observed using resting state functional magnetic resonance imaging (Greicius, et al., Proceedings of the National Academy of Sciences of the United States of America 101, 4637-4642, (2004)), and may also contribute to abnormal reaction time performance, a prominent feature of AD (Christ et al., Front Hum Neurosci 12, 124, (2018)) (FIG. 12).

### Example 1-6: Methods and Materials of Example 1

### Mice

5×FAD (34840-JAX, The Jackson Laboratory) mice were used. The genotyping of 5×FAD mice was carried out following the instructions provided by The Jackson Laboratory. All animal procedures were approved by the Institutional Animal Care and Use Committee at Yale University.

### Antibodies and Reagents

The following primary antibodies were used: anti-LAMP1 (DSHB, 1D4B), anti-GFP (Aves Labs. Inc. GFP-1020), anti-amyloid precursor protein (ThermoFisher Scientific, LN27, 13-0200), anti-PLD3 (Sigma-Aldrich, HPA012800), anti-MAP2 (Abcam, ab5392). All secondary antibodies used were conjugated with Alexa dyes from ThermoFisher Scientific. Thioflavin S (Sigma Aldrich, T1892) was used for staining amyloid plaques in fixed tissue. FSB (Santa Cruz, CAS 760988 Mar. 2) was used for labeling plaques in live mice.

### Adeno-Associated Virus (AAV) Production and Delivery

GCaMP6f and GCaMP6s viruses were purchased (UPenn Virus Core, AV-9-PV2822 and AV-9-PV2824; Addgene, #100837 and #100843). AAV vector for GFP overexpression were purchased from Addgene (#28014). AAV vector for tdTomato overexpression was constructed based on plasmid #28014 from Addgene mentioned above, in which the GFP sequence was deleted and replaced by the tdTomato sequence (sequence source http://www.tsienlab.ucsd.edu/Samples/PDF/tdTomato-map % 20&%20sequence.pdf, synthesized at Integrated DNA Technologies, the entirety of the file is hereby incorporated herein by reference).

All the constructs were verified by DNA sequencing, and expression or deletion of the target proteins was tested with immunohistochemistry of the target protein.

AAV2 vectors were produced and purified following the procedures described previously (Grimm et al., Mol Ther 7, 839-850 (2003)) using a two-plasmid helper free system (PlasmidFactory, Germany). Virus titer was determined by counting infection on HEK293 cells. AAV vectors were injected into the subarachnoid space in one hemisphere as previously described (Yuan et al., J Neurosci 36, 632-641 (2016)). Total viral particles injected per mouse were approximately 10E7.

### Cranial Window Implant

8-month-old 5×FAD mice were anesthetized with ketamine/xylazine solution (100 mg/kg and 10 mg/kg, respectively) and hair was removed on the skull area. Buprenex (0.1 mg/kg), dexamethasone (2 mg/kg) and carprofen (5 mg/kg) was given subcutaneously at this point. The mouse was put on a heating pad during the surgery and anesthesia was checked periodically. Povidone-iodine solution was applied on the skin and cleaned with ethanol. And eye ointment was applied on the eyes. A small piece of skin was removed to expose skull, and the membrane tissue on the skull surface was removed by forceps. A 4 mm diameter circle was drilled on the contralateral hemisphere of virus infusion (rough location of the center is −2.5 mm from Bregma and 2.5 mm from midline). The skull was rinsed with sterile PBS periodically to avoid excessive heating. Skull was thinned in a circumferential area and then lifted with fine forceps without causing injury to the underlying pila surface. Gelfoam sponge (Pfizer Inc.) was used to absorb blood after lifting the skull. Using a pair of very fine forceps, the dura was removed within the circle area and a 4-mm cover glass was gently pressed on the brain surface and glued to the skull. A customized head-bar was glued (for acute imaging) or chronically implanted (with dental cement, for chronic imaging) on the skull. For chronic imaging, mice were put on a heating pad to recover after the surgery and given daily of buprenex (0.1 mg/kg) and carprofen (5 mg/kg) for 3 days. Imaging procedures started one month after the surgery.

### In Vivo Two-Photon Imaging

Two-photon imaging was performed with a two-photon microscope equipped with a Ti-sapphire tunable laser (Spectra Physics), a Gallium arsenide phosphide (GaAsP) detector (Prairie technology) and a 20× water immersion objective (N.A. 1.0, Leica), or the Ultima Investigator multi-photon microscope (Bruker) with Insight X3 tunable ultrafast laser (Spectra Physics) and a 20× water immersion objective (N.A. 1.0, Olympus). GFP was excited at 920 nm; dTomato and tdTomato were excited at 920 nm/1045 nm; and FSB were excited at 850 nm. For chronic imaging, a location close to the center of the cranial window was selected as starting point and the blood vessel pattern was recorded. The coordinates of each region of interests were recorded as well. To relocate in the next imaging session, the starting point was relocated based on the recorded coordinates, and the field of view was adjusted to match the recorded blood vessel pattern.

### Spontaneous and Stimulated Calcium Imaging

6-8 months 5×FAD mice were injected with GCaMP6 virus through the subarachnoid space on one hemisphere to label cortical neurons and measure local axonal conduction properties. For measurements of interhemispheric axonal conduction, GCaMP6f virus were injected stereotaxically with the following coordinates: Bregma (AP:-0.34, ML: 1.65, DV: 0.45,: 0) (Oh et al., Nature 508, 207-214 (2014)) (Allen Mouse Brain Connectivity Atlas (2011)). After more than two weeks of the injection, an acute cranial imaging window was implanted on the contralateral hemisphere as described above. For stimulated calcium imaging, an additional opening on the skull was made on the ipsilateral side of the virus infusion. A glass electrode was inserted through this opening using a motorized micromanipulator and utilized for electrical stimulation.

The region of interest was located under a two-photon microscope. GCaMP6 were excited at 920 nm wavelength. Limited field of view was used to improve sampling rate. GCaMP6s was imaged at 2 Hz and GCaMP6f was imaged at 10 or 20 Hz. For imaging spontaneous activity, mice were imaged about three hours after initial ketamine/xylazine administration, and each axon was imaged for 10 minutes. Only axons with calcium transient at least once per minute were used for analysis. For stimulated calcium events, mice were anesthetized using 0.5% isoflurane. Spike trains of 2 ms pulses were delivered to the glass electrode at 50 Hz (18 ms interval) with 10 μA to 60 μA currents for 500 ms. The calcium response within the imaging window was monitored upon stimulation. The electrode was adjusted to different depths within the cortex, and the location that generated triggered responses in the axons of interest were used for experiments. Three consecutive trials of 5s or 10s imaging were acquired for each axon.

### Calcium Trace Analysis

The raw GCaMP6 fluorescence intensity was normalized to ΔF/F before analysis. Images were then spatially smoothed with a 3×3 window. Several regions of interests (ROIs) were selected on each axon. The average ΔF before stimulations were used as base line. For estimating the calcium rise time, the calcium trace from the event-specific peak to the first data point exceeding baseline was used as the rising phase of the event. This rising phase trace was fitted to an exponential equation: Y=1−exp (−k*(x−t)). The spike timing estimation (t0) was then calculated by extrapolating the x-intercept. For calculating the decay time constant, the decay phase of the curve (from the peak point to the end) was fitted with an exponential function, and the time constant was calculated as the decay time (the time that the intensity reduced to 1/e of the peak intensity). For analysis of the spontaneous Ca²⁺ transients, we calculated the correlation coefficients between two ROIs chose on each axonal side of the chosen spheroid using the Pearson correlation coefficient.

### Human Postmortem Brain Tissues

Formalin-fixed human postmortem brain tissue blocks were acquired from brain banks. Middle frontal gyrus, a cortical region affected in early stages of the disease, was used for this study. Detailed information can be found in FIGS. 11A-11C, including twelve AD cases and six mild cognitive impairment cases. Cases were matched for age, gender, and ApoE genotype. For immunohistochemistry of human tissue, 30 μm-thick slices were prepared and treated with sodium citrate solution at 95 degrees for 45 minutes, before staining with primary antibodies for 3 days.

### Computational Modeling

All modeling experiments of axon spheroids utilize methodologies previously validated (Morse et al., Front Neural Circuits 4 (2010)). The chosen morphology parameters and ion channel distributions were held constant within each compartment for each simulation. The axon length was 566 microns (μm), and diameters were set from 0.1 to 0.9 μm. The spheroids were modeled as a “cylinder and stick”. The stick (5 μm length and diameter varying from 0.3 to 8.3 μm) was connected to the middle of the axon and on the other end to the cylinder (a bulb head) whose surface area varied from 0 to 7100 μm² (equivalent spherical diameter varied from 0 to 150 μm). In most simulations, the axon contained voltage gated sodium and potassium channels and a leak current. The densities of the voltage gated channels were varied from 0 to an amount 1.1 times as strong as needed to produce regenerative (sustained) action potentials in the axon. Channel conductances were set in the spheroids with the same (varying) strength as was present in the axon. One or more strong current injection(s) (0.2 ms 2 nA) was applied to one end of the axon to reliably evoke a single or multiple (input) AP(s) that subsequently propagated to the spheroids. Current injections just over threshold generated qualitatively similar results (larger diameter axons required more current to evoke an AP). We checked for the presence of (output) APs on the other side of the spheroids by testing for the voltage exceeding 10 mV. We counted the number of output APs that were present in a 10 second simulation where either the single input AP occurred at, or the train of 20 Hz input APs began at 100 ms. On publication the NEURON simulation environment (Hines et al., Neuroscientist 7, 123-135 (2001)) computer code will be made available at ModelDB: modeldb dot yale dot edu/187612, the entirety of which is hereby incorporated herein by reference.

### Statistics

For analyzing difference in calcium rise time between region of interests (ROIs) along axon segments, the number of axons was used as sample size. This is because individual in vivo experiments had very few axons measured due to the necessary sparse labeling method used in these experiments. For analyzing inter-hemispheric calcium rise time delay, both the number of axons and mice were used as sample size. In all graphs, individual data points were shown. For analyzing spheroid number per plaque in postmortem human brains, the specific numbers of plaques measured from an individual human for can be found in the figure legend, and the average results were used as the representative outcome for that individual. The number of postmortem human tissues was used as sample size. In all statistical comparisons, non-parametric tests were used unless otherwise justified. Specific tests used for each graph can be found in the corresponding figure legend. When more than two groups were considered and compared, corrections for multiple comparisons were performed as part of the post-hoc analysis. All statistics were calculated using GraphPad Prism software.

### Example 2: Abnormally Enlarged Multivesicular Bodies Drives PAAS Expansion and is Modulated by PLD3

In Example 1 it was demonstrated that plaque-associated axonal spheroids (PAAS) lead to severe disruption in interhemispheric neural connectivity through blocks of action potential (AP) propagation. The size of axonal spheroid is a major driver for axonal conduction defects: smaller PAAS causes fewer AP blocks, while larger PAAS causes more AP blocks. Thus, modulation of spheroid size could potentially retore the disruption in neural connectivity caused by these structures. To elucidate the cellular and molecular mechanisms of PAAS enlargement, the subcellular structures within axonal spheroids were extensively characterized using a variety of imaging tools, including high-resolution confocal imaging, expansion microscopy and electron microscopy.

### Example 2-1: Accumulation of Abnormally Enlarged Multivesicular Bodies is Associated With Spheroid Expansion and Cognition Decline

Confocal microscopy revealed that as mice aged, there was a progressive accumulation of aberrantly enlarged LAMP-1 (lysosome associated membrane protein 1)-positive vesicles within PAAS (FIGS. 13A-13B). In addition, there was a striking correlation between the presence of enlarged LAMP-1 positive vesicles and the overall size of individual PAAS (FIG. 13C). Electron microscopy (EM) imaging revealed that these large vesicles were densely filled with smaller intraluminal vesicles (FIG. 13D), consistent with the ultrastructural characteristics of multivesicular bodies (MVBs), which are considered precursors of mature lysosomes. It was also found that small PAAS were predominantly filled with vesicles that contained high levels of the protease cathepsin D and were acidic (as measured with the pH-sensitive genetically encoded reporter SEpHluorin), which are characteristics of lysosomes (FIGS. 13E-13H). However, as PAAS enlarged, the overall acidification and cathepsin D levels in individual PAAS declined (FIGS. 13E-13H), coinciding with the accumulation of MVBs, which typically have not yet acquired lysosomal proteases and acidic pH. This indicates that spheroid enlargement is mechanistically linked with the accumulation of enlarged MVBs in mice.

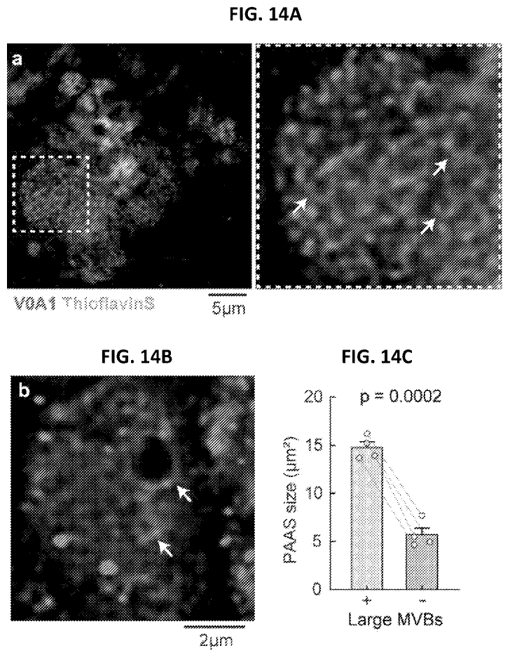

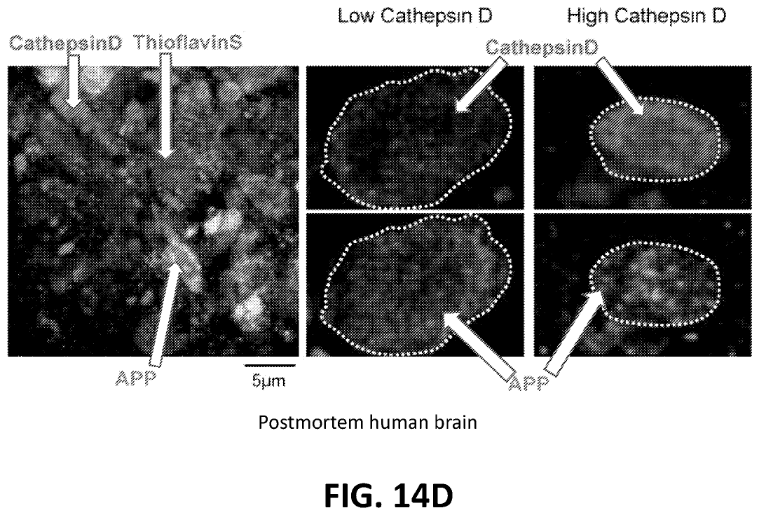

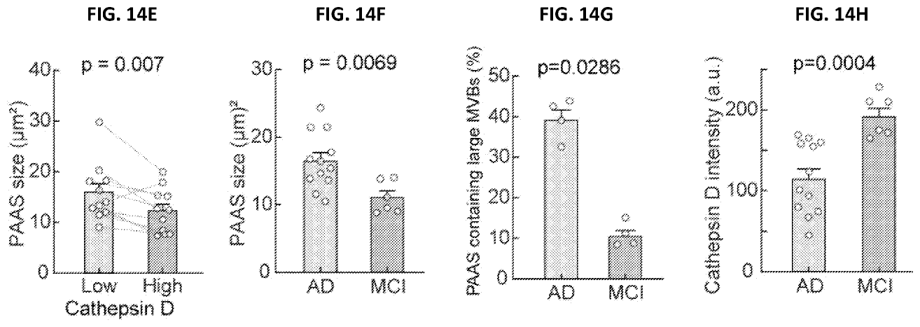

Similar MVB-like structures were observed within spheroids surrounding amyloid plaques in postmortem human brains (FIGS. 14A and 14B), and their presence was associated with larger spheroid size (FIG. 14C). In accordance with the abundance of immature MVBs in humans, larger PAAS contained lower levels of cathepsin D (FIGS. 14D and 14E). Importantly, consistent with the observation that PAAS size is inversely correlated with premortem cognitive function (FIG. 14F), a similar correlation between premortem cognition, the abundance of large MVBs and low levels of cathepsin D within PAAS was found (FIGS. 14G-14I). These data support a hypothesis in which accumulation of MVBs drives the enlargement of axonal spheroids, which disrupts axonal conduction and ultimately affects cognition.

### Example 2-2: PLD3 Mediates Multivesicular Body Enlargement and Spheroid Expansion

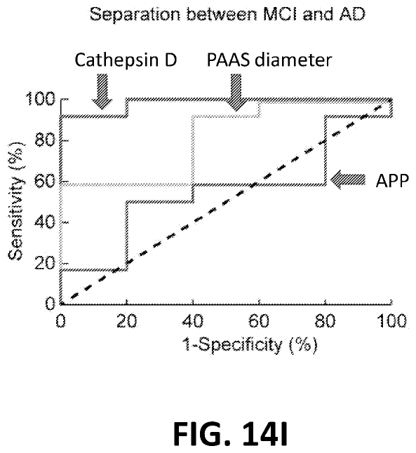

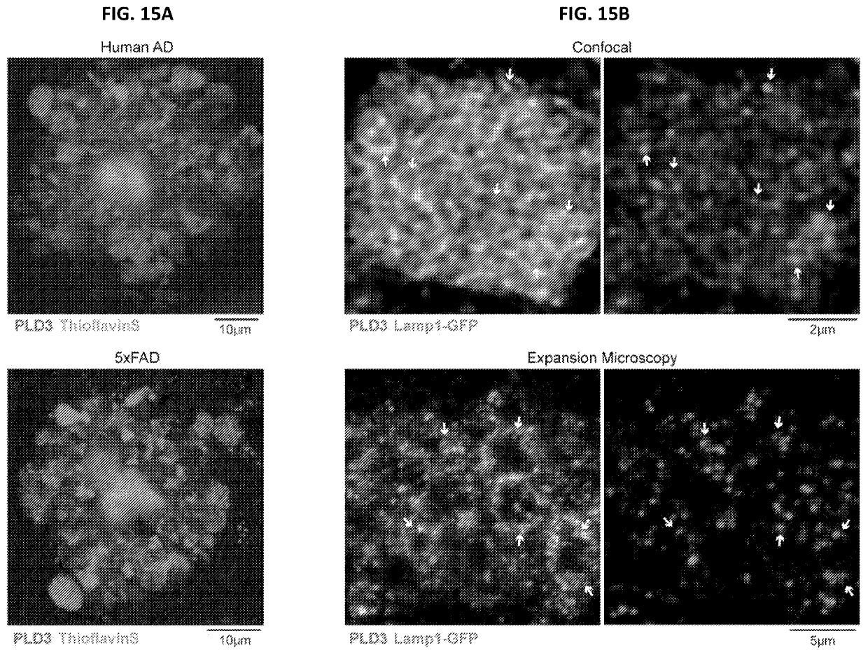

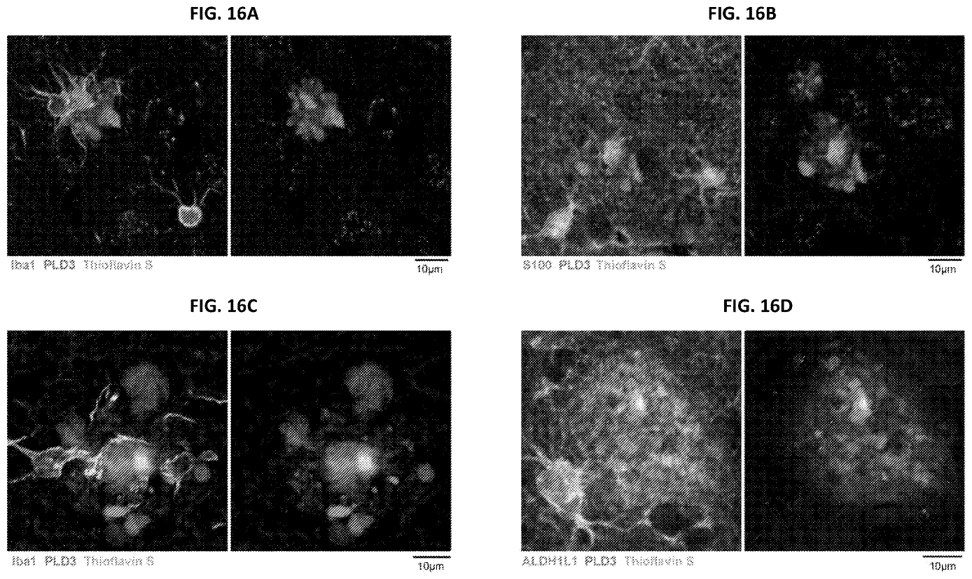

The potential mechanisms of accumulation of MVBs within axonal spheroids were then explored. PLD3 is a lysosomal protein that is of potential interest because it strongly accumulates in axonal spheroids in both humans and mice (FIG. 15A) and its expression is not detectable in other cell types such as microglia and astrocytes (FIGS. 16A-16D). In addition, PLD3 genetic variants may increase the risk of AD, although this remains a topic of controversy. Interestingly, PLD3 is considered to be the only lysosomal resident protein that is sorted into intraluminal vesicles (ILVs) of MVBs in mammals, in contrast to the majority of lysosomal resident proteins which are sorted to the limiting membranes of MVBs. Indeed, immunofluorescence confocal imaging of axonal spheroids showed accumulation of PLD3 within the lumen of LAMP-1 positive vesicular structures (FIG. 15B, upper panels), and expansion microscopy (ExM) revealed a punctate signal within MVBs, consistent with PLD3 positive ILVs (FIG. 15B, lower panels), similar to previous immunogold electron microscopy of cultured cells. Together, these observations raise the possibility that PLD3 may play a role in the formation of MVBs, thereby affecting their accumulation and the consequent enlargement of axonal spheroids.

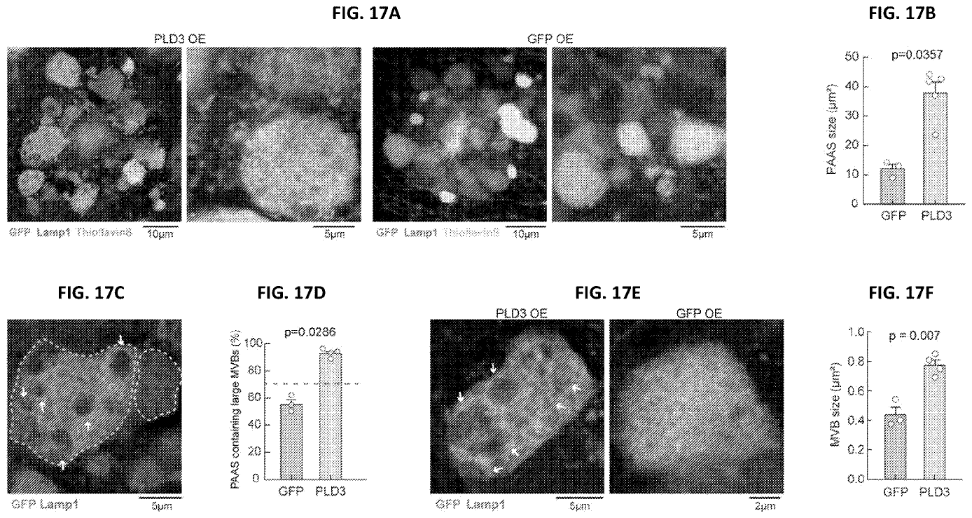

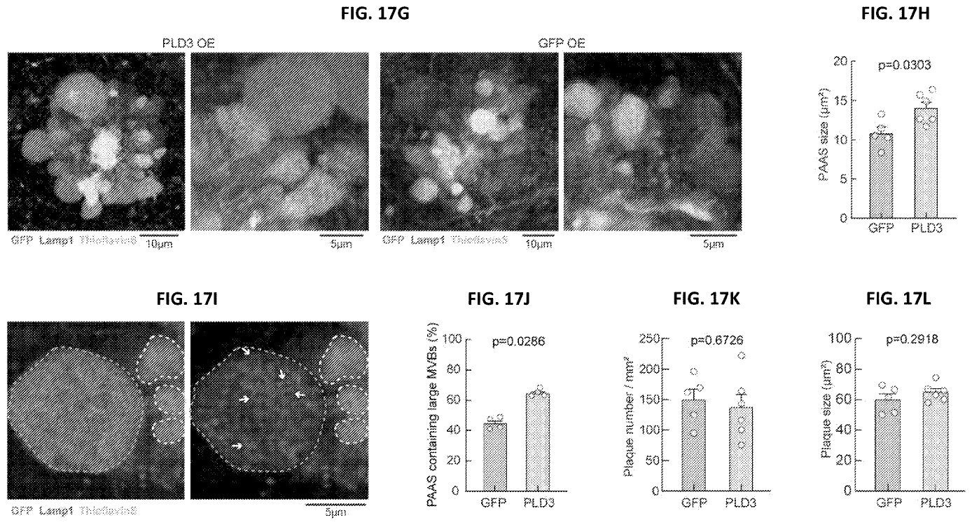

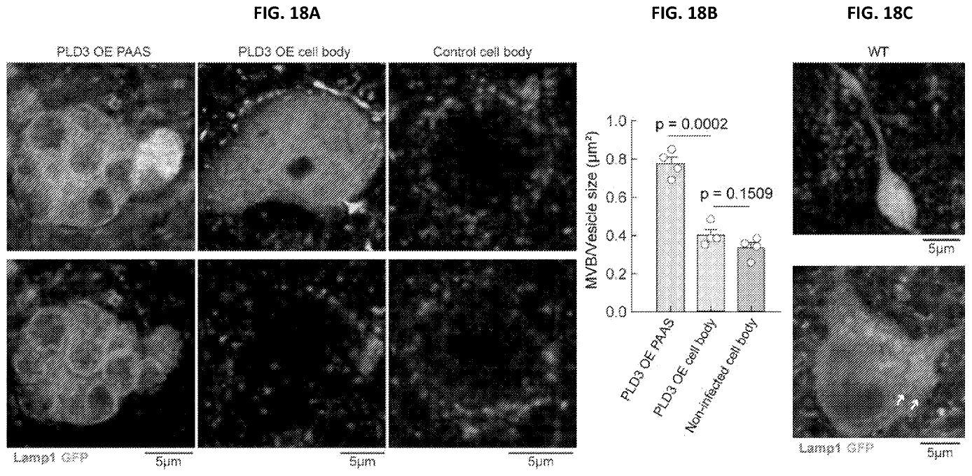

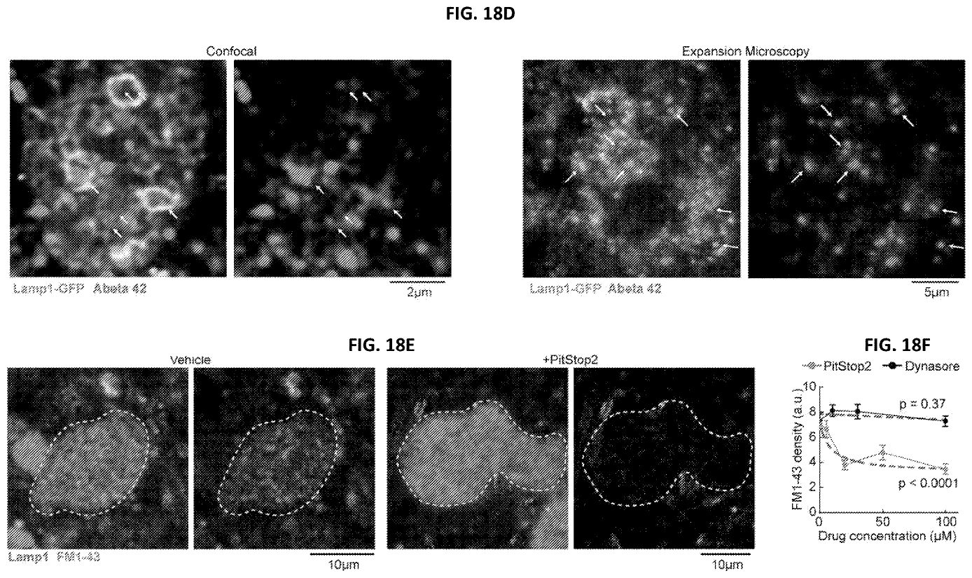

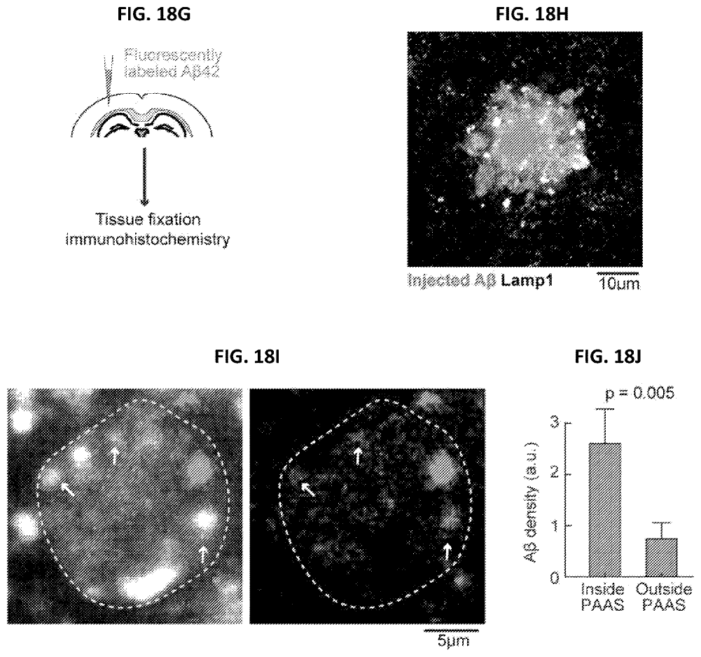

To further understand the role of PLD3 in the evolution of axonal spheroid pathology, in vivo AAV2-mediated overexpression of PLD3 in neurons of 5×FAD mice was implemented. It was observed that spheroids in PLD3 overexpressing axons were markedly larger than those expressing only GFP (Green Fluorescent Protein) (FIGS. 17A-17B and 17G-17H). Notably, confocal microscopy of individual spheroids revealed an increase in the number of large MVBs, even beyond what is seen in advanced aging in 5×FAD mice (FIGS. 17C-17D and 17I-17J). Furthermore, there was a marked increase in MVB size within PLD3 overexpressing spheroids compared to GFP-expressing controls (FIGS. 17E-17F). This manipulation was not associated with changes in amyloid plaque number or size (FIGS. 17K and 17L), indicating that PLD3 overexpression does not affect amyloid precursor protein (APP) processing. The enlargement and accumulation of large LAMP1-positive vesicles within PLD3 overexpressing spheroids, which are uniformly in the vicinity of amyloid plaques, was much greater than within cell bodies that were distant from plaques (FIGS. 18A-18B). This indicates that Aβ from extracellular deposits is critical for PLD3-induced MVB abnormalities in axons. Indeed, using an antibody that specifically recognizes the Aβ-42 peptide, it was observed that Aβ accumulation within large MVBs (FIG. 18D), where it could potentially synergize with PLD3. The source of Aβ-42 within MVBs is likely to be endocytosis of oligomeric peptides from adjacent amyloid plaques. This is supported by the data showing that spheroids are sites of very active endocytosis (FIGS. 18E-18F), and that administration of fluorescently labeled Aβ-42 to 5×FAD mice, leads to robust uptake into vesicular structures within spheroids (FIGS. 18G-18J). PLD3 overexpression in wildtype mice also led to occasional small axonal swellings with enlarged LAMP1-positive vesicles (FIG. 18C), indicating that excessive PLD3 by itself can have detrimental effects independently of amyloidosis. Altogether, these data indicates that the accumulation of PLD3 within PAAS, observed in both mice and humans, is mechanistically linked with MVB abnormalities and the subsequent enlargement of axonal spheroids, which is compounded by the concurrent effect of PLD3 and Aβ accumulation within the same subcellular compartments.

### Example 2-3: CRISPR/Cas9-Mediated PLD3 Deletion Reduces PAAS Size and Improves Axonal Conduction

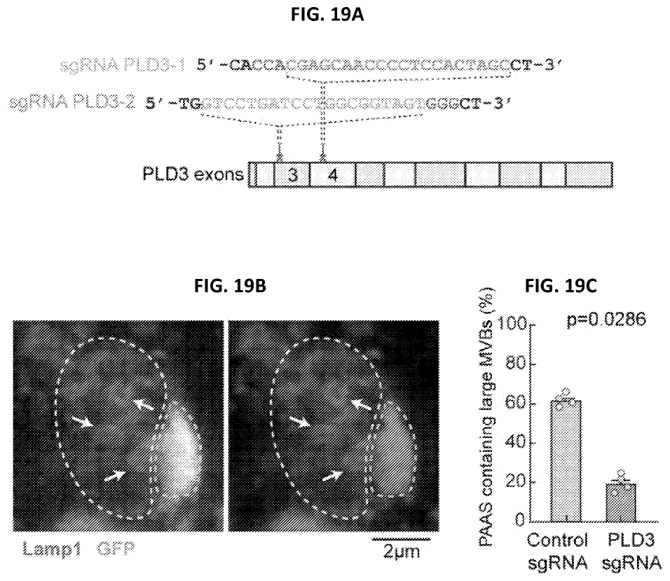

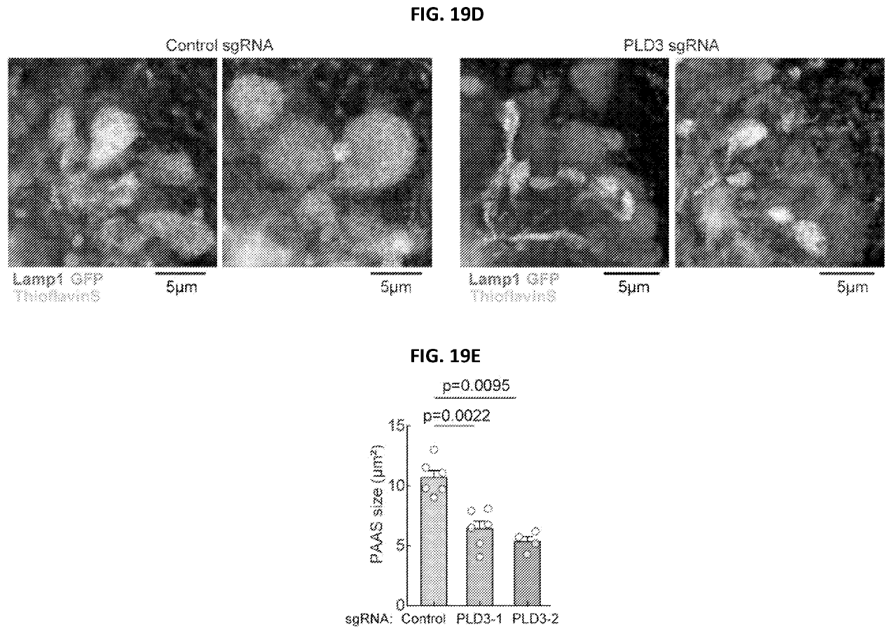

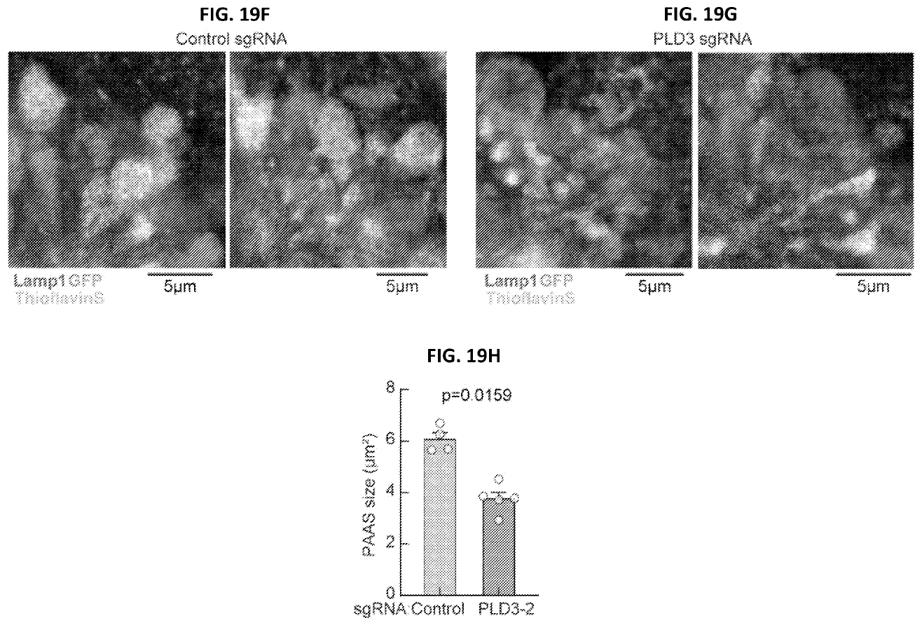

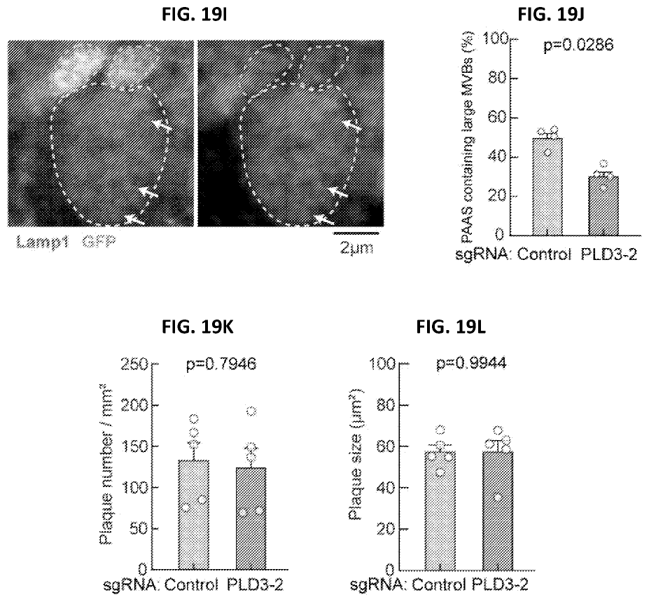

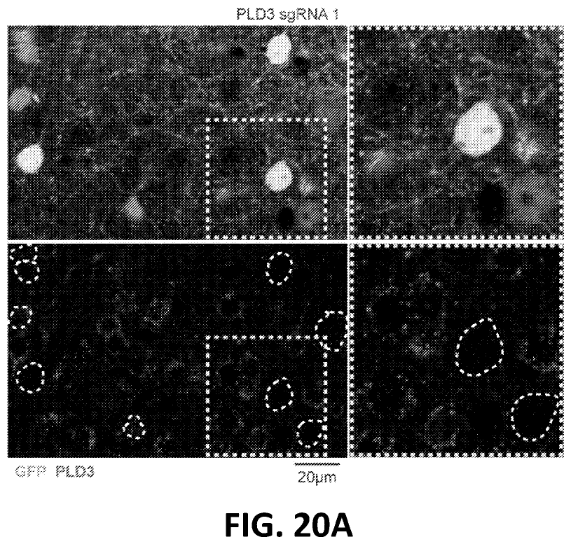

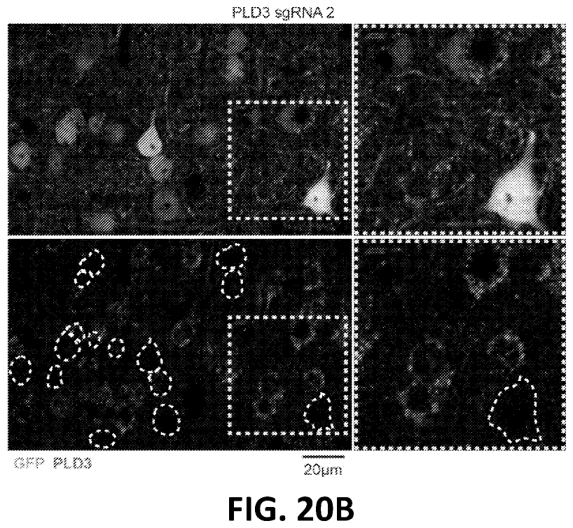

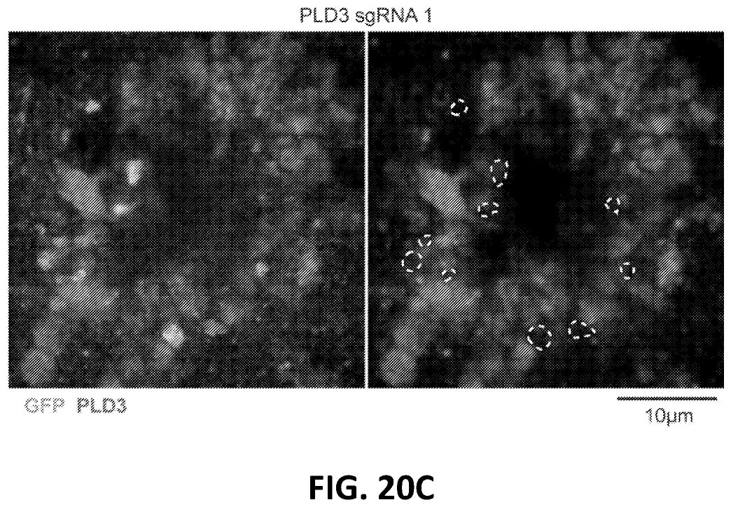

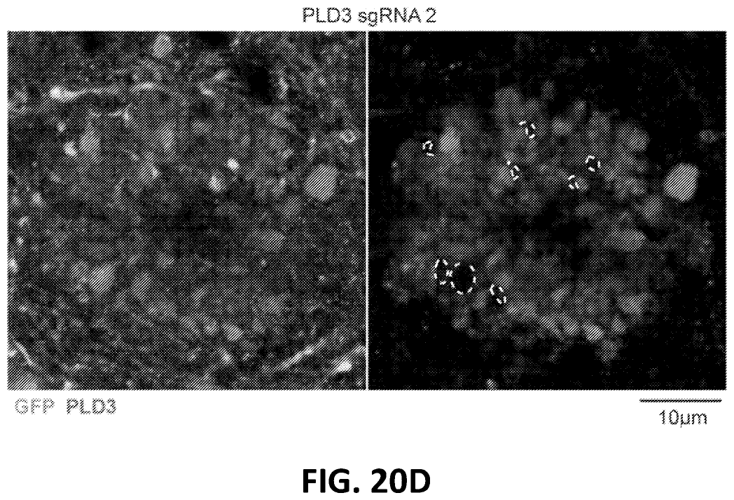

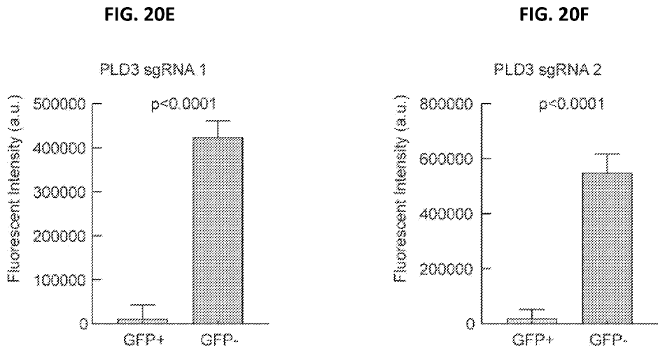

To test whether reducing PLD3 levels would ameliorate axonal spheroid pathology, the present study deleted PLD3 in neurons by AAV2-mediated CRISPR/Cas9 knockout in 5×FAD mice, using either of two single guide RNAs (sgRNAs) targeting different PLD3 exons (FIGS. 19A and 20A-20F). It was found that treatment with both sgRNAs led to a marked decrease in the abundance of large MVBs (FIGS. 19β-19C and 19I-19J) that was associated with an overall reduction in PAAS size, regardless of whether the treatment was initiated at 3 or 7 months of age in 5×FAD mice (FIGS. 19D-19H). This data demonstrates that PLD3 deletion at early or later stages of amyloid deposition can decrease MVB accumulation in axons, leading to a marked attenuation in spheroid enlargement, without any changes in amyloid plaque number or size (FIGS. 19K-19L).

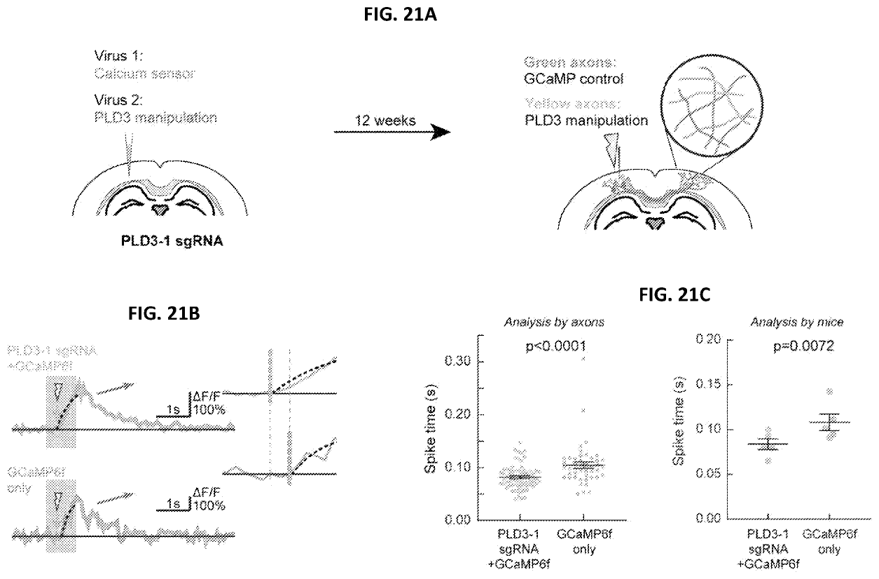

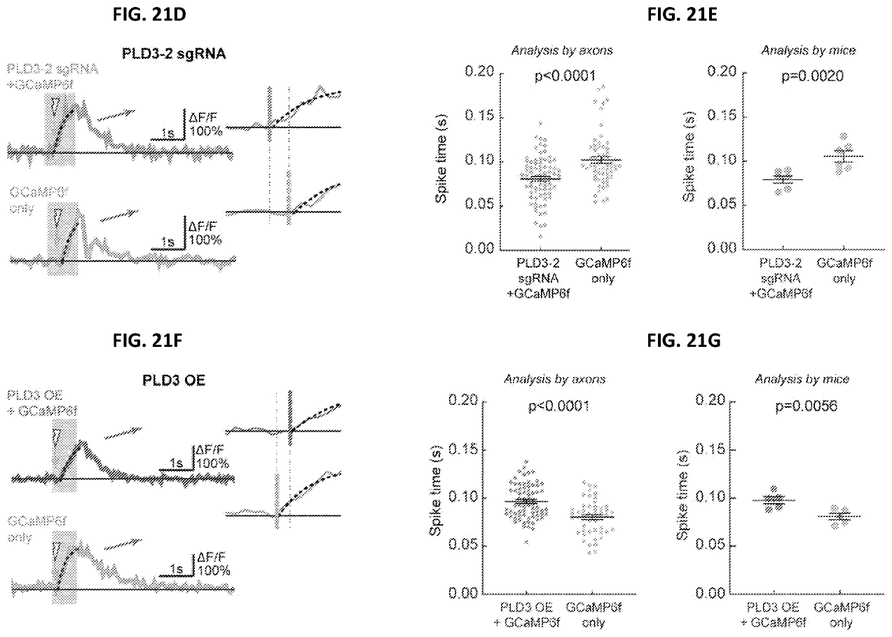

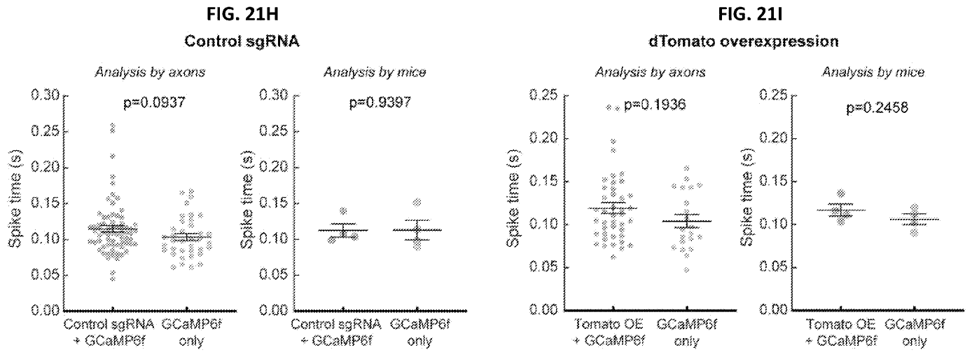

To test whether deletion of PLD3 in neurons and the consequent reduction in spheroids size has a beneficial effect on axonal conduction, neurons were co-infected with AAV2-U6-sgRNA (PLD3)-CAG-Tomato-P2A-Cre to delete PLD3 and AAV9-Syn-GCamP6f to implement the Ca²⁺ imaging approach for measuring interhemispheric axonal conduction (FIG. 21A). This co-infection strategy allowed the present study to image adjacent axons with or without PLD3 deletion within the same mouse and compare the Ca²⁺ rise times in contralateral cortex, following electrical stimulation of the ipsilateral hemisphere (FIG. 21A). Using the two sgRNAs, it was found that axons with PLD3 deletion, had a marked improvement in the propagation of APs (FIGS. 21B-21E and 21H) that approached what is seen in control non-AD mice (FIGS. 10B-10C). In contrast, PLD3 overexpression had the opposite effect, with a slowing of conduction velocity observed in axons with increased PLD3, compared to adjacent controls (FIGS. 21F-21G and 21I). Together these data demonstrates that PLD3 reduction can reverse MVB abnormalities in axons near plaques, leading to reduced spheroid size and restored axonal conduction properties.

To examine the impact of restoring conduction defects associated with axonal spheroids on neural circuit function, the present study focused on the basal forebrain (BF) nucleus of Meynert, which is a major source of cholinergic brain neurotransmission with extensive projections to the cortex and has been shown to degenerate in early stages of AD. Cortical projections from this nucleus are known to release acetylcholine to primarily activate interneurons and exert an overall inhibitory effect. Given the critical importance of this cholinergic circuit in cognitive function, the present study investigated the potential network effects of conduction deficits associated with PAAS and their potential reversibility by PLD3 modulation.

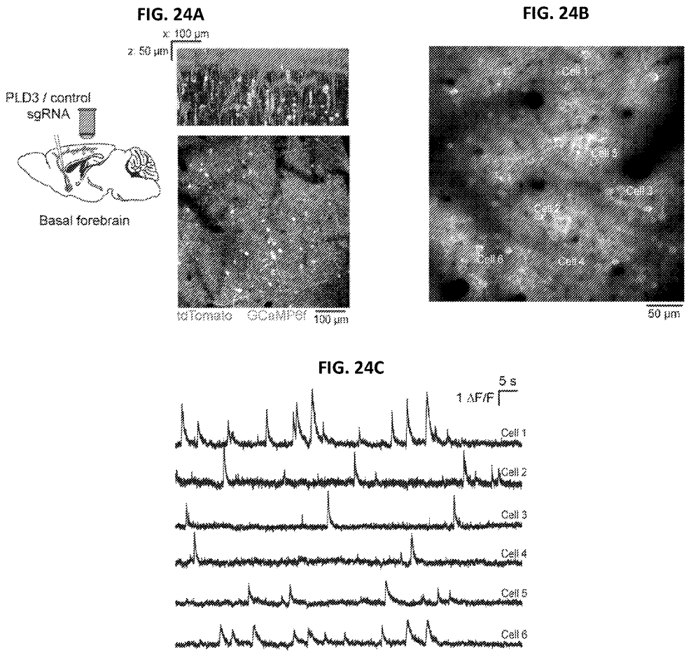

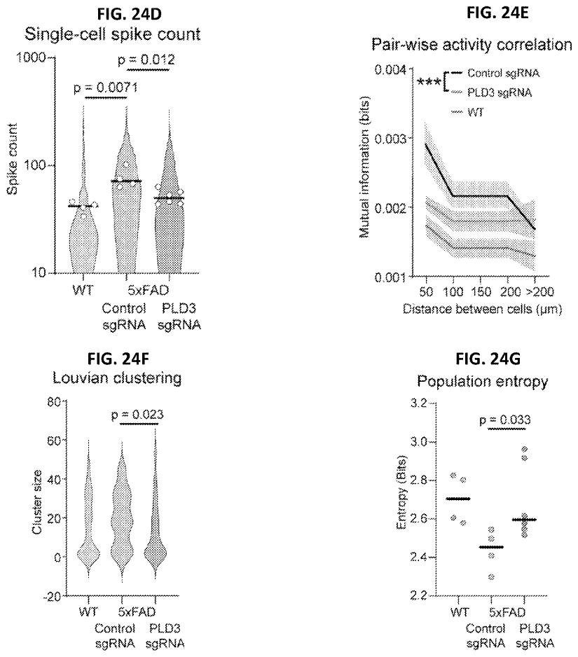

To achieve this, AAV2-U6-sgRNA (PLD3)-CAG-Tomato-P2A-Cre was injected to delete PLD3 in neurons of the basal forebrain in 7-month-old 5×FAD mice. To examine the effect of improved basal forebrain neurotransmission, the present study imaged spontaneous Ca²⁺ transients during awake resting sessions in neurons of layer ⅔ of the somatosensory cortex, previously infected with AAV9-Syn-GCamP6f (FIGS. 24A-24C). These neurons were within the immediate vicinity of projecting axons from basal forebrain (FIG. 24A, right panel). A higher proportion of hyperactive neurons in 5×FAD mice were observed (FIG. 24D). In addition, it was also found that 5×FAD mice showed increased correlated activity in neurons that were in close vicinity to each other (FIG. 24E), and displayed activity patterns with higher spatial-temporal similarities (FIGS. 24F-24G). These aberrant patterns of activity are predicted to markedly disrupt efficient information encoding. Importantly, PLD3 deletion in basal forebrain neurons led to a reduction in these aberrant activity patterns in cortical neurons to levels that were similar to WT controls (FIG. 24D-24G). Altogether, these data demonstrate that reducing action potential blockades in basal forebrain projection axons, which potentially improves neuromodulatory neurotransmission, can lead to a marked improvement on abnormalities in the patterns of activity of downstream cortical neurons.

### Example 2-4: Exploration of Other Molecular Targets in the Endosomal-Lysosomal-Autophagic System

In the previous sections it was demonstrated that modulation of MVB biogenesis by PLD3 reduced PAAS size and restored axonal conduction deficits. On the other hand, spheroid enlargement may also be targeted through modulation of other molecules involved in the endosomal-lysosomal-autophagic system. The present study thus explored the effects of other proteins involved in the endocytic-lysosomal-autophagic network on PAAS pathology.

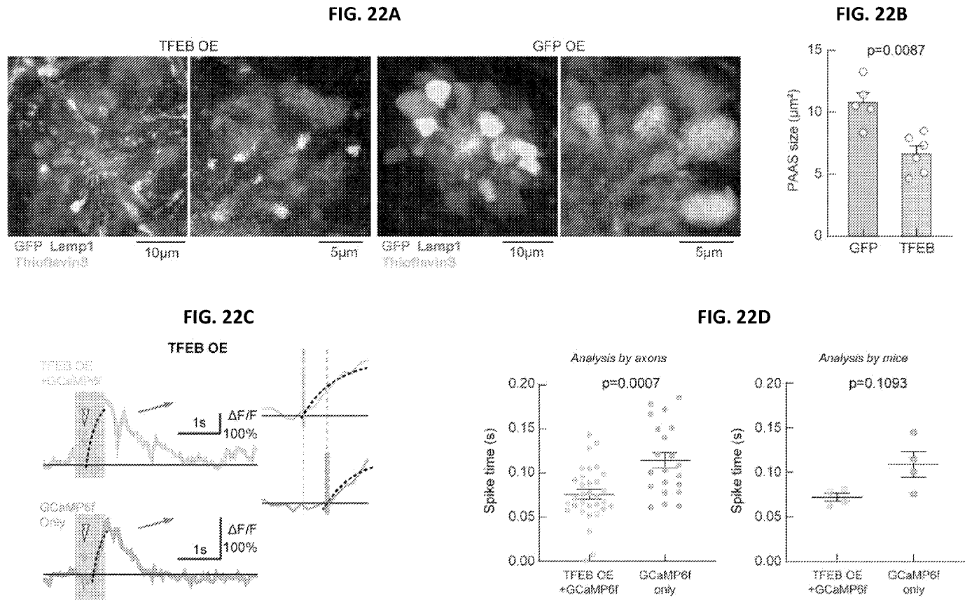

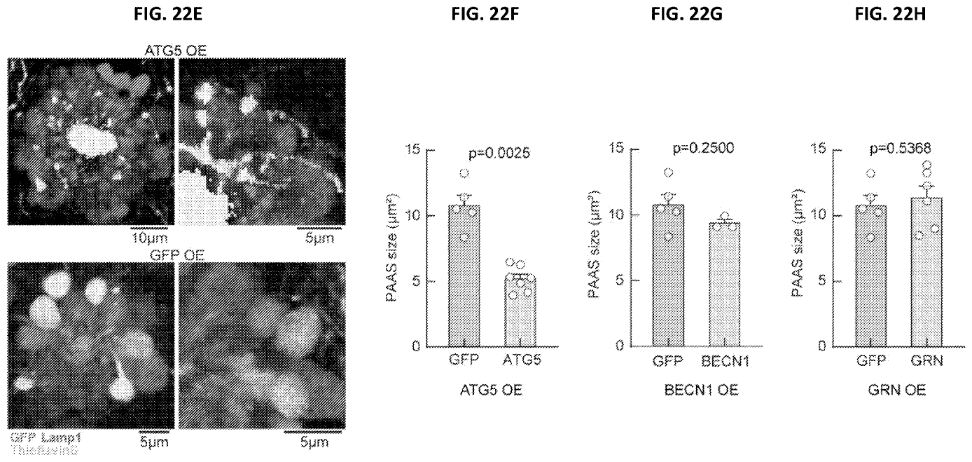

The transcription factor EB (TFEB) is a master regulator for lysosomal biogenesis and autophagy. Overexpression of TFEB leads to increased lysosome number and higher levels of lysosomal enzymes, as well as increased autophagosome formation and autophagosome-lysosome fusion. The present study found that neuronal overexpression of TFEB leads to reduction in spheroid size (FIGS. 22A-22B). This indicates that enhancing lysosomal biogenesis and autophagy broadly could ameliorate the burden of vesicle accumulation within PAAS, leading to PAAS reduction. Furthermore, when neurons were co-infected with AAV2-CAG-Tomato-P2A-TFEB to overexpress TFEB and AAV9-Syn-GCamP6f to implement our Ca²⁺ imaging approach for measuring interhemispheric axonal conduction, it was found that TFEB overexpression also led to a marked improvement in axon conduction (FIGS. 22C-22D).

Autophagy-related vesicles were found in axonal spheroids. Overexpressing autophagy-related protein 5 (ATG5), a protein critical for autophagy, in neurons of 5×FAD mice led to a reduction in PAAS size (FIGS. 22E-22F). Interestingly, overexpression of Beclin 1, another protein involved in autophagy induction, did not result in changes in the size of spheroids (FIG. 22G). This might be because these two proteins are involved in different process in autophagy. ATG5 is critical for autophagic vesicle formation, elongation of isolation membrane and the fusion between autophagosomes and lysosomes. On the other hand, Beclin 1 is important in the nucleation stage of autophagy and is essential in the recruitment of ATG proteins to the isolation membrane. Thus, the promotion of autophagosome-lysosome fusion by ATG5 overexpression might facilitate the maturation of autophagosomes into lysosomes and bypass the fusion between autophagosome and abnormal MVBs, thereby ameliorating the burden of accumulating vesicles within PAAS, leading to reduction in PAAS size. Another possibility might be that Beclin 1 is unique among the ATG genes in the way that it is not only involved in autophagy, but also involved in other cellular processes. Therefore, manipulation of Beclin 1 might lead to other effects that counteracts with its autophagic effect.

The protein level of PLD3 was previously shown to be elevated in mice with deletion of the lysosomal protein Progranulin (GRN). GRN is a risk factor for frontotemporal dementia (FTD) and has also been associated with AD. Progranulin was also found to be enriched within PAAS. We thus tested the effect of GRN overexpression on spheroid enlargement and found that this manipulation did not affect PAAS size (FIG. 22H), indicating that GRN effect in AD may not be mediated through PAAS enlargement.

### Example 2-5

To explore the potential cellular and molecular mechanisms of PAAS enlargement, a variety of imaging tools were utilized. Including high-resolution confocal imaging, expansion microscopy and electron microscopy. It was found that abnormally enlarged multivesicular bodies (MVBs) accumulate in axonal spheroids and their presence correlates with spheroid size. In addition, it was found an increased presence of large MVBs within spheroids in older 5×FAD mice and in more severely impaired human AD patients, indicating that MVB accumulation may be a key feature of disease progression. Since spheroids in AD only develop in the vicinity of amyloid plaques, the MVB abnormalities that the present study found are likely to be amyloid dependent. Interestingly, the present observed robust endocytic activity at axonal spheroids, which was associated with uptake of Aβ-42 oligomers into endosomal compartments within PAAS. Thus, internalization of Aβ from extracellular deposits may trigger endo-lysosomal abnormalities in axons. Indeed, Aβ-42 was present within MVBs at axonal spheroids, consistent with previous immunogold electron microscopy showing that in AD patients, the most prominent subcellular localization of Aβ-42 is within MVBs. These data indicate a mechanistic link between the accumulation of MVBs, due to their impeded maturation into lysosomes around plaques, and the subsequent enlargement of PAAS, which disrupt normal axonal conduction.

The endosomal sorting complex required for transport (ESCRT) machinery plays a major role in MVB biogenesis by regulating the formation of intraluminal vesicles (ILVs) within MVBs and the sorting of proteins into ILVs destined for degradation. In contrast to lysosomal resident proteins sorted to the limiting membrane of MVBs, PLD3, a potential risk factor for AD, is the only resident protein in mammals known to be sorted into ILVs through the ESCRT pathway. Indeed, the present study found that PLD3 was present within ILVs of large MVBs and was highly enriched in axonal spheroids. This indicates that PLD3 may play a role in neuronal MVB maturation. Consistent with this, the present study found that overexpression of PLD3 in neurons led to a marked enlargement and accumulation of MVBs and resulted in an overall increase in PAAS size. These data established a potential causal link between PLD3 accumulation in spheroids, MVB abnormalities and subsequent PAAS enlargement.

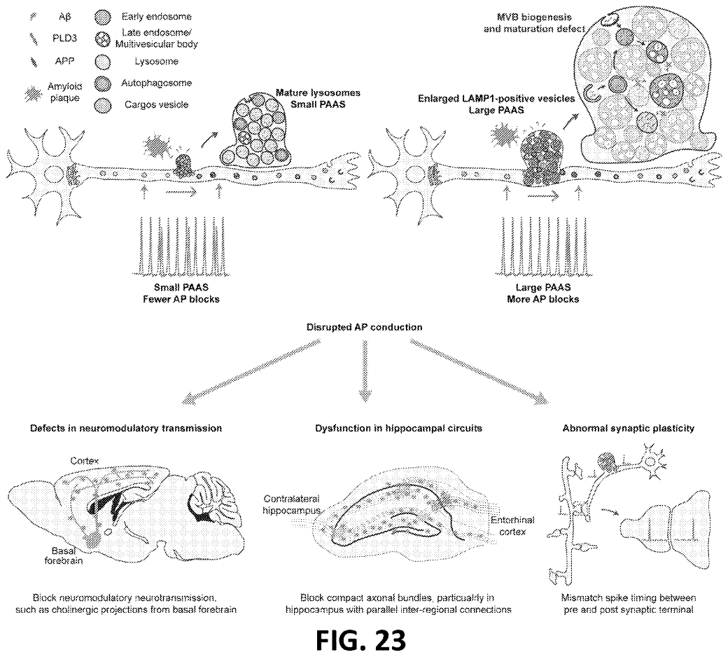

Deletion of ESCRT components can lead to enlarged endosomal compartments. Given the accumulation of PLD3 in axonal spheroids, it is possible that PLD3 leads to MVB enlargement by interfering with ESCRT machinery. In line with this, in wildtype mice, PLD3 overexpression led to enlargement of LAMP1-positive vesicles and formation of small axonal swellings. However, the dramatic enlargement of MVBs following PLD3 overexpression, predominantly occurs in PAAS which are in the vicinity of amyloid plaques. This indicates that extracellular Aβ is also critical for PLD3-induced MVB enlargement. Consistent with this, administration of Aβ-42 to cultured neurons has been shown to result in MVB enlargement. Together with the observation that Aβ-42 is actively endocytosed into PAAS and present within MVBs, PLD3 could work synergistically with Aβ-42 in the same subcellular compartment, leading to greater MVB abnormalities. On the other hand, given that APP and β-site amyloid precursor protein cleaving enzyme (BACE1) accumulate within PAAS, the present study do not exclude that intracellularly produced Aβ could also contribute to abnormalities in MVB biogenesis. Interestingly, APP is also sorted into ILVs of MVBs through the ESCRT machinery, and deletion of ESCRT components promotes APP processing and increased intracellular Aβ. Thus, PLD3 and Aβ may constitute a vicious cycle in which axonal endocytosis and/or intracellularly produced Aβ, facilitates PLD3-induced MVB enlargement and accumulation (FIG. 23).

Given the finding that PLD3 overexpression increases PAAS size and worsens axonal conduction, it was hypothesized that reducing PLD3 would have a beneficial effect. Indeed, the present study found that CRISPR/Cas9 neuronal knockout of PLD3 led to a reduction in the abundance of enlarged MVBs within spheroids and a decrease in PAAS size. Importantly, this led to a marked decrease in the frequency of axonal conduction blocks, thus improving interhemispheric connectivity. The effects of PLD3 deletion on PAAS size were seen in early as well as late stages of amyloidosis, indicating the potential for prevention as well as reversal of pre-existing spheroids.

It is worth noting that other proteins in the endo-lysosomal pathway could lead to changes in PAAS size, as well. Indeed, neuronal overexpression of either TFEB or ATG5 lead to a reduction in spheroid size. However, given the negligible expression of PLD3 in non-neuronal cells (FIG. 16), this molecule could be a promising therapeutic target because global modulation of the endo-lysosomal pathway may negatively affect glial cells and their roles in controlling protein aggregation and amyloid brain accumulation. Thus, Modulation of MVB biogenesis through PLD3 or other endo-lysosomal molecules could thus constitute a novel strategy for ameliorating PAAS pathology, independent of amyloid plaque removal.

FIG. 23 depicts a non-limiting model of PAAS enlargement and functional consequences in Alzheimer's disease. The present study demonstrated that the accumulation of abnormally enlarged MVBs is a major driver of PAAS enlargement. Small PAAS predominately contain mature lysosomes, while bigger PAAS contain abundant and enlarged MVBs. The present study identified PLD3 as a critical modulator of MVB abnormalities and subsequent spheroid enlargement. PLD3 is uniquely sorted through the ESCRT pathway into the intralumenal vesicles (ILVs) of MVBs. Accumulation of PLD3 at spheroids could lead to MVB enlargement by interfering with ESCRT machinery. This process could be exacerbated with the presence of Aβ. Aβ from extracellular amyloid deposits is actively endocytosed and is present in the same subcellular compartments as PLD3. PLD3 could thus work synergistically with Aβ, leading to greater MVB abnormalities. Large PAAS cause more severe conduction blocks, by functioning as electrical capacitors that act as current sinks. Given that hundreds of axons around each plaque develop spheroids and these structures remain stable for extended periods of times, the large number of plaques present in the AD brain could significantly affect neural networks by widespread disruption of axonal connectivity. Parallel compact axonal bundles that follow a stereotyped projection path along a tri-synaptic loop in hippocampus, a region critical for memory formation, could be particularly vulnerable to amyloid plaques located in the region. Furthermore, neural processes that rely on temporally precise long-range coordination among brain regions, such as memory consolidation, could be severely affected. In addition, synaptic plasticity could also be disrupted, due to the requirement of precise timing of firing between pre-synaptic and post-synaptic terminals. Altogether, action potential blocks caused by PAAS could be detrimental to various neural processes such as memory formation and reaction time, potentially contributing to cognitive decline in AD.

### Example 2-6: Methods and Materials of Example 2

### Mice

5×FAD (34840-JAX, The Jackson Laboratory) mice were used. Rosa26-LSL-Cas9 (026175, The Jackson Laboratory) mice were crossed with 5×FAD for CRSIPR/Cas9-mediated gene deletion. The genotyping of 5×FAD mice was carried out following the instructions provided by The Jackson Laboratory. All animal procedures were approved by the Institutional Animal Care and Use Committee at Yale University.

### Antibodies and Reagents

The following primary antibodies were used in this study: anti-LAMP1 (DSHB, 1D4B), anti-GFP (Aves Labs. Inc. GFP-1020), anti-CathepsinD (Abcam, EPR3057Y, ab75852), anti-ATP6V0A1 (ThermoFisher Scientific, PA5-54570), anti-amyloid precursor protein (ThermoFisher Scientific, LN27, 13-0200), anti-PLD3 (Sigma-Aldrich, HPA012800), anti-beta amyloid 1-42 (Abcam, ab10148), anti-beta amyloid 1-42 (Abcam, mOC98, ab201061), anti-MAP2 (Abcam, ab5392), anti-Ibal (Novus Biologicals, NB100-1028), anti-S100B (R&D Systems, AF1820), anti-Aldhlll (NeuroMab, P28037), FM1-43 (Life Technologies, F35355). All secondary antibodies used were conjugated with Alexa dyes from ThermoFisher Scientific. Thioflavin S (Sigma Aldrich, T1892) was used for staining amyloid plaques in fixed tissue. FSB (Santa Cruz, CAS 760988 Mar. 2) was used for labeling plaques in live mice. To study spheroid endocytosis of extracellular Aβ, fluorescently labeled Aβ1-42 (AnaSpec) was used. Pitstop2 (Abcam, ab120687) was used to inhibit endocytosis.

### Adeno-Associated Virus (AAV) Production and Delivery

GCaMP6f and GCaMP6s viruses were purchased (UPenn Virus Core, AV-9-PV2822 and AV-9-PV2824; Addgene, #100837 and #100843). AAV vector for GFP overexpression were purchased from Addgene (#28014). Customized AAV vectors for overexpression were constructed based on plasmid #28014 from Addgene, in which the GFP sequence was deleted and replaced by the customized sequences described below. In many cases where the virus transduced both a target protein and a fluorescent protein reporter, a GFP without the stop codon and P2A sequence was placed in front of the target protein sequence in the same open reading frame, as described previously (Yuan et al., J Neurosci 36, 632-641 (2016)). The target proteins used in this study are:

mCherry-SEpHluorin: sequence was cut from Addgene #32001;

LAMP1-GFP: LAMP-1 sequence was amplified from mouse brain mRNA, using the 5′ primer TGCGTCGCGCCATGGCGGCC (SEQ ID NO: 1) and 3′ primer GATGGTCTGATAGCCGGCGT (SEQ ID NO: 2);

GFP-P2A-PLD3: PLD3 sequence was amplified from mouse PLD3 cDNA (GE open biosystem), using the 5′ primer ATGAAGCCCAAACTGATGTACCAGG (SEQ ID NO: 3) and 3′ primer TCAAAGCAGGCGGCAGGC (SEQ ID NO: 4).

The sgRNA constructs for PLD3 deletion were cloned using plasmid #60229 from Addgene. The sequences of the sgRNAs are:

PLD3 sgRNA 1:
(SEQ ID NO: 5)
GTCCTGATCCTGGCGGTAGT;
PLD3 sgRNA 2:
(SEQ ID NO: 6)
GCTAGTGGAGGGGTTGCTCG;
Control sgRNA:
(SEQ ID NO: 7)
GGAAGAGCGAGCTCTTCT.

All the constructs were verified by DNA sequencing, and expression or deletion of the target proteins was tested with immunohistochemistry of the target protein.

AAV2 vectors were produced and purified following the procedures described previously (Grimm et al., Mol Ther 7, 839-850 (2003)) using a two-plasmid helper free system (PlasmidFactory, Germany). Virus titer was determined by counting infection on HEK293 cells. For AAV-mediated treatments, AAV viruses were injected into the subarachnoid space in one hemisphere as previously described (Yuan et al., J Neurosci 36, 632-641 (2016)). Total viral particles injected per mouse were approximately 10E7. For AAV-mediated labeling of subcellular structures, AAV viruses were injected stereotaxically with the following coordinates: Bregma (AP:-0.34, ML: 1.65, DV: 0.45,: 0) (Allen Mouse Brain Connectivity Atlas (2011)).

### Cranial Window Implant

8-month-old 5×FAD mice were anesthetized with ketamine/xylazine solution (100 mg/kg and 10 mg/kg, respectively) and hair was removed on the skull area. Buprenex (0.1 mg/kg), dexamethasone (2 mg/kg) and carprofen (5 mg/kg) was given subcutaneously at this point. The mouse was put on a heating pad during the surgery and anesthesia was checked periodically. Povidone-iodine solution was applied on the skin and cleaned with ethanol. And eye ointment was applied on the eyes. A small piece of skin was removed to expose skull, and the membrane tissue on the skull surface was removed by forceps. A 4 mm diameter circle was drilled on the contralateral hemisphere of virus infusion (rough location of the center is −2.5 mm from Bregma and 2.5 mm from midline). The skull was rinsed with sterile PBS periodically to avoid excessive heating. Skull was thinned in a circumferential area and then lifted with fine forceps without causing injury to the underlying pila surface. Gelfoam sponge (Pfizer Inc.) was used to absorb blood after lifting the skull. Using a pair of very fine forceps, the dura was removed within the circle area and a 4-mm cover glass was gently pressed on the brain surface and glued to the skull. A customized head-bar was glued (for acute imaging) or chronically implanted (with dental cement, for chronic imaging) on the skull. For chronic imaging, mice were put on a heating pad to recover after the surgery and given daily of buprenex (0.1 mg/kg) and carprofen (5 mg/kg) for 3 days. Imaging procedures started one month after the surgery.

### In Vivo Two-Photon Imaging

Two-photon imaging was performed with a two-photon microscope equipped with a Ti-sapphire tunable laser (Spectra Physics), a Gallium arsenide phosphide (GaAsP) detector (Prairie technology) and a 20× water immersion objective (N.A. 1.0, Leica), or the Ultima Investigator multi-photon microscope (Bruker) with Insight X3 tunable ultrafast laser (Spectra Physics) and a 20× water immersion objective (N.A. 1.0, Olympus). GFP was excited at 920 nm; dTomato and tdTomato were excited at 920 nm/1045 nm; and FSB were excited at 850 nm. For chronic imaging, a location close to the center of the cranial window was selected as starting point and the blood vessel pattern was recorded. The coordinates of each region of interests were recorded as well. To relocate in the next imaging session, the starting point was relocated based on the recorded coordinates, and the field of view was adjusted to match the recorded blood vessel pattern.

### Stimulated Calcium Imaging

6-8 months 5×FAD mice were injected with GCaMP6 virus through the subarachnoid space on one hemisphere to label cortical neurons and measure local axonal conduction properties. For measurements of interhemispheric axonal conduction, GCaMP6f virus were injected stereotaxically with the following coordinates: Bregma (AP:-0.34, ML: 1.65, DV: 0.45,: 0) (Allen Mouse Brain Connectivity Atlas (2011)). After more than two weeks of the injection, an acute cranial imaging window was implanted on the contralateral hemisphere as described above. An additional opening on the skull was made on the ipsilateral side of the virus infusion. A glass electrode was inserted through this opening using a motorized micromanipulator and utilized for electrical stimulation.

The region of interest was located under a two-photon microscope. GCaMP6f were excited at 920 nm wavelength. Limited field of view was used to improve sampling rate. GCaMP6f was imaged at 10 or 20 Hz. Mice were anesthetized using 0.5% isoflurane. Spike trains of 2 ms pulses were delivered to the glass electrode at 50 Hz (18 ms interval) with 10 μA to 60 μA currents for 500 ms. The calcium response within the imaging window was monitored upon stimulation. The electrode was adjusted to different depths within the cortex, and the location that generated triggered responses in the axons of interest were used for experiments. Three consecutive trials of 5s or 10s imaging were acquired for each axon.

### Calcium Trace Analysis

The raw GCaMP6 fluorescence intensity was normalized to ΔF/F before analysis. Images were then spatially smoothed with a 3×3 window. Several regions of interests (ROIs) were selected on each axon. The average ΔF before stimulations were used as base line. For estimating the calcium rise time, the calcium trace from the event-specific peak to the first data point exceeding baseline was used as the rising phase of the event. This rising phase trace was fitted to an exponential equation: Y=1−exp (−k*(x−t)). The spike timing estimation (t0) was then calculated by extrapolating the x-intercept.

### Human Postmortem Brain Tissues

Formalin-fixed human postmortem brain tissue blocks were acquired from brain banks. Middle frontal gyrus, a cortical region affected in early stages of the disease, was used for this study. Detailed information can be found in FIGS. 11A-11C, including twelve AD cases and six mild cognitive impairment cases. Cases were matched for age, gender, and ApoE genotype. For immunohistochemistry of human tissue, 30 μm-thick slices were prepared and treated with sodium citrate solution at 95 degrees for 45 minutes, before staining with primary antibodies for 3 days.

### Plaque-Associated Axonal Spheroids Imaging and Quantification

Fixed tissue imaging was performed with a confocal microscope (Leica SP5 or Lecia SP8), and the images were taken with a 63× oil objective (N.A. 1.4, Leica). Individual PAAS size were measured by manually selecting outlines of individual bulbs based on LAMP1, APP or V0A1 immunohistochemistry. Multivesicular body (MVB)+PAAS were defined as PAAS containing at least one LAMP1-positive ring structure. For classifying PAAS into neutral/acidic (using the genetically encoded pH sensor) and cathepsin D low/high groups, arbitrary thresholds of green/red fluorescence intensity of 0.75 and cathepsin D immunostaining fluorescence intensity of 100 were used using NIH imageJ/Fuji software.

### Expansion Microscopy

Expansion of brain sections were performed following conventional immunostaining. Brain sections were treated with Glutaraldehyde (GA; TCI Chemicals, G0068) and then subjected to gelation, digestion and expansion as described previously (Chen et al., Science 347, 543-548 (2015) and Tillberg et al., Nature biotechnology 34, 987-992 (2016)). Briefly, brain sections were first incubated with monomer solution (1×PBS, 2 M NaCl, 8.625% (w/w) sodium acrylate, 2.5% (w/w) acrylamide, 0.15% (w/w) N,N′-methylenebisacrylamide) at 4° C. for 45 min. Then transferred into a gel chamber and incubated in gelling solution (concentrated stocks (10% w/w) of ammonium persulfate (APS) initiator and tetramethyl-ethylenediamine (TEMED) accelerator added to the monomer solution for up to 0.2% (w/w) each and the inhibitor 4-hydroxy-2,2,6,6-tetramethylpiperidin-1-oxyl(4-hydroxy-TEMPO) added up to 0.01% (w/w) from a 0.5% (w/w) stock) at 37° C. for 1.5-2 hours for gelation. The gels were then fully immersed in proteinase solution (Proteinase K (New England Biolabs, P8107S) diluted 1:100 to 8 units/mL in the digestion buffer (50 mM Tris (pH 8), 1 mM EDTA, 0.5% Triton X-100, 1 M NaCl)) at 37° C. overnight. Digested gels were next placed in excess volumes of double deionized water (ddH₂O) for 25 min to expand. This step was repeated 3-5 times in ddH₂O, until the size of the expanding sample plateaued.

### Transmission Electron Microscopy

12-month-old 5×FAD mice were perfused with 4% PFA and the brain tissues were sectioned into 50 μm-thick slices with Vibratome (VT1000S, Leica). The slices were re-fixed in 2% glutaraldehyde in 0.1M cacodylate buffer (pH 7.4) for 1 hour, then post-fixed in 1% OsO4 in the same buffer at room temperature for 1 hour. After en bloc staining with 2% aqueous uranyl acetate for 30 min, tissue was dehydrated in a graded series of ethanol to 100%, followed by propylene oxide and finally embedded in EMBed 812 resins. Tissue blocks were polymerized in 60° C. oven overnight. Thin sections (60 nm) were cut by a Leica ultramicrotome (UC7) and post-stained with 2% uranyl acetate and lead citrate. Sample grids were examined in a FEI Tecnai transmission electron microscope with accelerating voltage of 80 kV, digital electron micrographs were recorded with an Olympus Morada CCD camera and iTEM imaging software.

### AAV-Mediated Molecular Manipulations

AAV vectors were injected into ~3-month-old and ~7-month-old 5×FAD mice. AAVs were infused through subarachnoid space. Brain tissues were collected ~1.5 months and ~3 months after virus injection for treatments initiated at 3 and 7 months of age, respectively, and fixed with 4% paraformaldehyde. Brain slices of 50 μm thickness were prepared and stained with anti-LAMP1 antibody (DSHB, 1D4B) and Thioflavin S.

Imaging and quantification of PAAS were carried out. Briefly, tiled Z-stack images of the infected cortical regions were taken at zoom 1 at 1 μm Z-steps. Individual plaques were segmented from the tiled images and blinded for the mouse and treatment information. For measurement of PAAS bulb size, the Z plane with the largest cross-section area of each individual spheroid was selected, and the cross-section area was measured using NIH Image J/Fiji software by manually selecting outlines of that cross-section based on virally expressed cytoplasmic GFP fluorescence.

### Statistics

For analyzing MVB presence, cathepsin D level and pH level in the axon spheroids, and spheroid bulb size after treatment, the specific numbers of plaques or spheroids measured from an individual human or mouse for each experiment can be found in each figure legend, and the average results were used as the representative outcome for that individual. The number of postmortem human tissues or mice was used as sample size in these cases. For analyzing difference in calcium rise time between region of interests (ROIs) along axon segments, the number of axons was used as sample size. This is because individual in vivo experiments had very few axons measured due to the necessary sparse labeling method used in these experiments. For analyzing inter-hemispheric calcium rise time delay, both the number of axons and mice were used as sample size. In all graphs, individual data points were shown. In all statistical comparisons, non-parametric tests were used unless otherwise justified. Specific tests used for each graph can be found in the corresponding figure legend. When more than two groups were considered and compared, corrections for multiple comparisons were performed as part of the post-hoc analysis. All statistics were calculated using GraphPad Prism software.

The present study demonstrated that axonal spheroid growth was driven by an age-dependent accumulation of large multivesicular bodies (MVBs) and was mechanistically linked with the endo-lysosomal protein PLD3, which is highly enriched in spheroids. Modulation of neuronal MVB biogenesis through PLD3 could potentially reverse neural network abnormalities in AD, independently of amyloid removal. In Example 3 the earlier stages of PAAS formation will be investigate and the underlying cellular and molecular mechanisms will be elucidated.

### Enumerated Embodiments

In some aspects, the present invention is directed to the following non-limiting embodiments:
Embodiment 1: A method of treating, ameliorating, and/or preventing a neurodegenerative condition in a subject in need thereof, the method comprising
   administering to the subject a compound that reverses, ameliorates, and/or prevents formation or enlargement of an axonal spheroid in a neuron of the subject.
Embodiment 2: The method of Embodiment 1, wherein the neurodegenerative condition is at least one selected from the group consisting of Alzheimer's disease, Lou Gehrig's disease (ALS), Huntington's disease, post traumatic encephalopathy, Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, and/or Lewy Body dementia.
Embodiment 3: The method of Embodiment 1, wherein the subject is a human.
Embodiment 4: The method of Embodiment 1, wherein the compound is a compound that: downregulates an activity and/or expression level of phospholipase D3 (PLD3) in a neuron affected by the neurodegenerative condition; upregulates an activity and/or expression level of transcription factor EB (TFEB) in a neuron affected by the neurodegenerative condition; and/or upregulates an activity and/or expression level of autophagy-related protein 5 (ATG5) in a neuron affected by the neurodegenerative condition.
Embodiment 5: The method of Embodiment 4, wherein the downregulates the activity and/or expression level of PLD3, and the compound comprises at least one selected from the group consisting of:
   a small molecule inhibitor of PLD3,
   a protein inhibitor of PLD3,
   a nucleic acid that downregulates the expression level and/or activity of PLD3 by RNA interference, and/or an expression vector expressing the nucleic acid that downregulates the expression level and/or activity of PLD3 by RNA interference,
   a ribozyme that downregulates the expression level and/or activity of PLD3, and/or an expression vector expressing the ribozyme,
   an expression vector comprising an expression cassette, wherein the expression cassette expresses CRISPR components that downregulate the expression level and/or activity of PLD3 by CRISPR knockout or CRISPR knockdown, and
   a trans-dominant negative mutant protein of PLD3, and/or an expression vector that expresses the trans-dominant negative mutant protein of PLD3.
Embodiment 6: The method of Embodiment 5, wherein the compound comprises at least one selected from the group consisting of: the expression vector expressing the ribozyme, the expression vector comprising an expression cassette expressing the CRISPR components, and the expression vector that expresses the trans-dominant negative mutant protein, and wherein the expression vector comprises a viral vector.
Embodiment 7: The method of Embodiment 6, wherein the expression vector comprises an adeno-associated virus (AAV).
Embodiment 8: The method of Embodiment 1, further comprises administering to the subject a compound that removes a protein aggregate in the brain of the subject.
Embodiment 9: A method of reversing, ameliorating or preventing a formation and/or enlargement of an axonal spheroid, the method comprising:
   contacting a neuron affected by the formation or enlargement of the axonal spheroid with a compound that:
      downregulates an activity and/or expression level of phospholipase D3 (PLD3) in a neuron;
      upregulates an activity and/or expression level of transcription factor EB (TFEB) in a neuron; and/or
      upregulates an activity and/or expression level of autophagy-related protein 5 (ATG5) in a neuron.
Embodiment 10: The method according to Embodiment 9, wherein the axonal spheroid blocks or delays a propagation of an action potential (AP) along an axon of the neuron.
Embodiment 11: The method according to Embodiment 9, wherein the formation and/or enlargement of axonal spheroids is associated with a neurodegenerative condition in a subject.
Embodiment 12: The method according to Embodiment 11, wherein the neurodegenerative condition is at least one selected from the group consisting of Alzheimer's disease, Lou Gehrig's disease (ALS), Huntington's disease, post traumatic encephalopathy, Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, and Lewy Body dementia.
Embodiment 13: The method according to Embodiment 12, wherein the neurodegenerative condition is Alzheimer's disease, and the axonal spheroid is associated with an amyloid plaque.
Embodiment 14: The method according to Embodiment 9, wherein the compound that downregulates the expression level or the activity of PLD3 comprises at least one selected from the group consisting of:
   a small molecule inhibitor of PLD3,
   a protein inhibitor of PLD3,
   a nucleic acid that downregulates the expression level and/or activity of PLD3 by RNA interference, and/or an expression vector expressing the nucleic acid that downregulates the expression level and/or activity of PLD3 by RNA interference,
   a ribozyme that downregulates the expression level and/or activity of PLD3, and/or an expression vector expressing the ribozyme,
   an expression vector comprising an expression cassette, wherein the expression cassette expresses CRISPR components that downregulate the expression level and/or activity of PLD3 by CRISPR knockout or CRISPR knockdown, and
   a trans-dominant negative mutant protein of PLD3, and/or an expression vector that expresses the trans-dominant negative mutant protein of PLD3.
Embodiment 15: A pharmaceutical composition for treating a neurodegenerative condition in a subject, the pharmaceutical composition comprising:
   a compound that:
      downregulates an activity and/or expression level of phospholipase D3 (PLD3) in a neuron affected by the neurodegenerative condition;
      upregulates an activity and/or expression level of transcription factor EB (TFEB) in a neuron affected by the neurodegenerative condition; and/or
      upregulates an activity and/or expression level of autophagy-related protein 5 (ATG5) in a neuron affected by the neurodegenerative condition; and
      a pharmaceutically acceptable carrier.
Embodiment 16: The pharmaceutical composition of Embodiment 15, wherein the neurodegenerative condition is at least one selected from the group consisting of Alzheimer's disease, Lou Gehrig's disease (ALS), Huntington's disease, post traumatic encephalopathy, Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, and Lewy Body dementia.
Embodiment 17: The pharmaceutical composition of Embodiment 15, wherein the compound comprises at least one selected from the group consisting of:
   a small molecule inhibitor of PLD3,
   a protein inhibitor of PLD3,
   a nucleic acid that down regulates the expression level and/or activity of PLD3 by RNA interference, and/or an expression vector expressing the nucleic acid that downregulates the expression level and/or activity of PLD3 by RNA interference,
   a ribozyme that downregulates the expression level and/or activity of PLD3, or an expression vector expressing the ribozyme,
   an expression vector comprising an expression cassette, wherein the expression cassette expresses CRISPR components that downregulate the expression level and/or activity of PLD3 by CRISPR knockout or CRISPR knockdown, and
   a trans-dominant negative mutant protein of PLD3, or an expression vector that expresses the trans-dominant negative mutant protein of PLD3.
Embodiment 18: The pharmaceutical composition of Embodiment 17, wherein the compound comprises at least one selected from the group consisting of: the expression vector expressing the ribozyme, the expression vector comprising an expression cassette expressing the CRISPR components, and the expression vector that expresses the trans-dominant negative mutant protein, and wherein the expression vector comprises a viral vector.
Embodiment 19: The pharmaceutical composition of Embodiment 15, wherein the subject is a human.
Embodiment 20: The pharmaceutical composition of Embodiment 15, which further comprises a compound that removes a protein aggregate from the subject's brain.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. A method of treating, or ameliorating a neurodegenerative condition in a subject in need thereof, the method comprising
administering to the subject a compound that reverses, ameliorates, or prevents formation or enlargement of an axonal spheroid in a neuron of the subject.

2. The method of claim 1, wherein the neurodegenerative condition is at least one selected from the group consisting of Alzheimer's disease, Lou Gehrig's disease (ALS), Huntington's disease, post traumatic encephalopathy, Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, and Lewy Body dementia.

3. The method of claim 1, wherein the subject is a human.

4. The method of claim 1, wherein the compound is a compound that:
downregulates an activity or expression level of phospholipase D3 (PLD3) in a neuron affected by the neurodegenerative condition;
upregulates an activity or expression level of transcription factor EB (TFEB) in a neuron affected by the neurodegenerative condition; and/or
upregulates an activity or expression level of autophagy-related protein 5 (ATG5) in a neuron affected by the neurodegenerative condition.

5. The method of claim 4, wherein the downregulates the activity or expression level of PLD3, and the compound comprises at least one selected from the group consisting of:
a small molecule inhibitor of PLD3,
a protein inhibitor of PLD3,
a nucleic acid that downregulates the expression level or activity of PLD3 by RNA interference, or an expression vector expressing the nucleic acid that downregulates the expression level or activity of PLD3 by RNA interference,
a ribozyme that downregulates the expression level or activity of PLD3, or an expression vector expressing the ribozyme,
an expression vector comprising an expression cassette, wherein the expression cassette expresses CRISPR components that downregulate the expression level or activity of PLD3 by CRISPR knockout or CRISPR knockdown, and
a trans-dominant negative mutant protein of PLD3, or an expression vector that expresses the trans-dominant negative mutant protein of PLD3.

6. The method of claim 5,
wherein the compound comprises at least one selected from the group consisting of: the expression vector expressing the ribozyme, the expression vector comprising an expression cassette expressing the CRISPR components, and the expression vector that expresses the trans-dominant negative mutant protein, and
wherein the expression vector comprises a viral vector.

7. The method of claim 6, wherein the expression vector comprises an adeno-associated virus (AAV).

8. The method of claim 1, further comprises administering to the subject a compound that removes a protein aggregate in the brain of the subject.

9. A method of reversing, ameliorating, or preventing a formation or enlargement of an axonal spheroid, the method comprising:
contacting a neuron affected by the formation or enlargement of the axonal spheroid with a compound that:
downregulates an activity or expression level of phospholipase D3 (PLD3) in a neuron;
upregulates an activity or expression level of transcription factor EB (TFEB) in a neuron; or
upregulates an activity or expression level of autophagy-related protein 5 (ATG5) in a neuron.

10. The method according to claim 9, wherein the axonal spheroid blocks or delays propagation of an action potential (AP) along an axon of the neuron.

11. The method according to claim 9, wherein the formation or enlargement of axonal spheroids is associated with a neurodegenerative condition in a subject.

12. The method according to claim 11, wherein the neurodegenerative condition is at least one selected from the group consisting of Alzheimer's disease, Lou Gehrig's disease (ALS), Huntington's disease, post traumatic encephalopathy, Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, and Lewy Body dementia.

13. The method according to claim 12, wherein the neurodegenerative condition is Alzheimer's disease, and the axonal spheroid is associated with an amyloid plaque.

14. The method according to claim 9, wherein the compound that downregulates the expression level or the activity of PLD3 comprises at least one selected from the group consisting of:
a small molecule inhibitor of PLD3,
a protein inhibitor of PLD3,
a nucleic acid that downregulates the expression level or activity of PLD3 by RNA interference, or an expression vector expressing the nucleic acid that downregulates the expression level or activity of PLD3 by RNA interference,
a ribozyme that downregulates the expression level or activity of PLD3, or an expression vector expressing the ribozyme,
an expression vector comprising an expression cassette, wherein the expression cassette expresses CRISPR components that downregulate the expression level or activity of PLD3 by CRISPR knockout or CRISPR knockdown, and
a trans-dominant negative mutant protein of PLD3, or an expression vector that expresses the trans-dominant negative mutant protein of PLD3.

15. A pharmaceutical composition for treating a neurodegenerative condition in a subject, the pharmaceutical composition comprising:
a compound that:
downregulates an activity or expression level of phospholipase D3 (PLD3) in a neuron affected by the neurodegenerative condition;
upregulates an activity or expression level of transcription factor EB (TFEB) in a neuron affected by the neurodegenerative condition; or
upregulates an activity or expression level of autophagy-related protein 5 (ATG5) in a neuron affected by the neurodegenerative condition; and
a pharmaceutically acceptable carrier.

16. The pharmaceutical composition of claim 15, wherein the neurodegenerative condition is at least one selected from the group consisting of Alzheimer's disease, Lou Gehrig's disease (ALS), Huntington's disease, post traumatic encephalopathy, Niemann-Pick disease type C, adult-onset leukoencephalopathy with axonal spheroids and pigmented glia (ALSP), hereditary leukoencephalopathy with axonal spheroids, Nasu-Hakola disease, Parkinsons's disease, and Lewy Body dementia.

17. The pharmaceutical composition of claim 15, wherein the compound comprises at least one selected from the group consisting of:
a small molecule inhibitor of PLD3,
a protein inhibitor of PLD3,
a nucleic acid that down regulates the expression level or activity of PLD3 by RNA interference, or an expression vector expressing the nucleic acid that downregulates the expression level or activity of PLD3 by RNA interference,
a ribozyme that downregulates the expression level or activity of PLD3, or an expression vector expressing the ribozyme,
an expression vector comprising an expression cassette, wherein the expression cassette expresses CRISPR components that downregulate the expression level or activity of PLD3 by CRISPR knockout or CRISPR knockdown, and
a trans-dominant negative mutant protein of PLD3, or an expression vector that expresses the trans-dominant negative mutant protein of PLD3.

18. The pharmaceutical composition of claim 17,
wherein the compound comprises at least one selected from the group consisting of: the expression vector expressing the ribozyme, the expression vector comprising an expression cassette expressing the CRISPR components, and the expression vector that expresses the trans-dominant negative mutant protein, and
wherein the expression vector comprises a viral vector.

19. The pharmaceutical composition of claim 15, wherein the subject is a human.

20. The pharmaceutical composition of claim 15, which further comprises a compound that removes a protein aggregate from the subject's brain.
